(19) United States
(12) Patent Application Publication
(10) Pub. No.: US 2007/0054275 A1
(43) Pub. Date: Mar. 8, 2007

(54) Method evolved for recognition of thrombophilia (mert)

(76) Inventors: Cigdem F. Dogulu, Bethesda, MD (US); Owen M. Rennert, Potomac, MD (US); Wal-Yer Chan, North Potomac, MD (US)

Correspondence Address: KLARQUIST SPARKMAN, LLP, 121 S.W. SALMON STREET, SUITE #1600, PORTLAND, OR, 97204-2988, US

(21) Appl. No.: 10/586,288

(22) PCT Filed: Jan. 14, 2005
(86) PCT No.: PCT/US05/01419

§ 371 Date: Jul. 13, 2006

Related U.S. Application Data
(60) Provisional application No. 60/537,463, filed on Jan. 15, 2004.

(51) Int. Cl. C12Q 1/68

(52) U.S. Cl. 435/6

(57) ABSTRACT

Methods for predicting an individual's genetic risk for developing venous thrombosis in diverse ethnic populations is disclosed, as are arrays and kits which can be used to practice the method. The method includes screening for mutations, polymorphisms, or both, in at least eight venous thrombosis-related molecules, such as antithrombin III, protein C, protein S, fibrinogen, factor V, prothrombin (factor II), methylenetetrahydrofolate reductase (MTHFR), and angiotensin I-converting enzyme (ACE) molecules which are associated with venous thrombosis.

### CROSS REFERENCE TO RELATED APPLICATION

This application claims the benefit of U.S. Provisional Application No. 60/537,463 filed Jan. 15, 2004, which is hereby incorporated by reference in its entirety.

### FIELD

This application relates to methods of predicting an individual's genetic susceptibility to venous thrombosis, as well as kits that can be used to practice the disclosed methods.

### BACKGROUND

Venous thrombosis affects 1 per 1000 individuals annually and is one of the leading causes of mortality and morbidity resulting in approximately 300,000 hospitalizations and 50,000 fatalities per year in the United States alone (Rosendaal, Thromb. Haemost. 78:1-6, 1997; Nordstrom et al., J. Inter. Med. 232:155-60, 1992; and Hansson et al., Arch. Intern. Med. 157:1665-70, 1997).

Numerous conditions predispose an individual to venous thrombosis. Examples of such risk factors include pregnancy, puerperium, use of oral contraceptives and/or hormone replacement therapy, trauma, surgery, fractures, prolonged immobilization, advanced age, antiphospholipid antibodies, previous thrombosis history, myeloproliferative disorders, malignancy, and mild-to-moderate hyperhomocysteinemia (Abramson et al., Southern Med. J. 94:1013-20, 2001; and Seligsohn and Lubetsky, N. Engl. J Med. 344:1222-31, 2001). Venous thrombosis often occurs in the lower leg as a deep venous thrombosis (DVT) which often leads to pulmonary emboli that are often fatal. If is particularly unfortunate that such thromboembolic phenomena often occur in already physiologically compromised patients.

In addition to acquired risk factors for venous thrombosis, a number of seemingly monogenic, autosomal dominant, variably penetrant genetic mutations or polymorphisms impart an increased risk for venous thrombosis. Examples of such mutations or polymporphisms are in genes that encode procoagulant proteins (Factor V, prothrombin and fibrinogen), natural anticoagulant proteins (protein C, protein S and antithrombin III and other thrombosis related proteins (angiotensin-I converting enzyme and methylenetetrahydrofolate reductase). Therefore, venous thrombosis is a complex genetic disorder. Genetic defects leading to hyperactivity of the coagulation system are present in a large proportion of patients with venous thrombosis. More than 60% of the predisposition to thrombosis is attributable to genetic components (Souto et al., Am. J. Hum. Genet. 67:1452-9, 2000).

Previous reports describe screening for one or more polymorphisms associated with thrombosis, for example by using PCR (Harrington et al., Clin. Chem. Lab. Med. 41:496-500, 2003), or microplate array diagonal gel electrophoresis (Bauer et al., Thromb. Haemost. 84:396-400, 2000). Although the use of microarray technology to screen for mutations in particular genes involved in venous thrombosis has been proposed, these microarrays are limited because they have a low predictive value and only detect mutations that are prevalent in Caucasian populations (for example see Pecheniuk et al., Blood Coagul. Fibrinolysis 11:683-700, 2000; Pollak et al., Ital. Heart J. 2:568-72, 2001; Evans and Lee-Tataseo, Clin. Chem. 48:1406-11, 2002; Schrijver et al., Am. J. Clin: Pathol. 119:490-6, 2003; Erali et al., Clin. Chem. 49:732-9, 2003). Others have indicated that microarray technology needs to undergo further development before it is available for screening the numerous genetic mutations and polymorphisms involved in thrombosis (Grody, Annu. Rev. Med., 54:473-90, 2003).

Therefore, there is a need for a method that can accurately predict the risk of an individual for developing venous thrombosis, which can be used to screen multiple ethnic populations.

### SUMMARY

Although venous thrombosis is one of the leading causes of morbidity and mortality in developed countries, it is an avoidable disease by the use of prophylactic treatment with currently available anticoagulants such as unfractionated heparin, low molecular-weight heparins, aspirin, and coumadin/warfarin. Thus, it is beneficial to estimate the individual thrombotic risk to develop stratification protocols for an individual risk-adapted prophylaxis to avoid the development of venous thromboembolism. For this stratification, the individual risk associated with single or combined risk factors of hemostasis is estimated.

The inventors have identified combinations of mutations and polymorphisms in venous thrombosis-related molecules that allow one to predict the genetic susceptibility of an individual to developing venous thrombosis with high accuracy in several ethnic populations. In one example, the combinations of recurrent mutations and polymorphisms in molecules that are statistically associated with venous thrombosis allow for prediction of the overall genetic susceptibility of an individual to developing venous thrombosis with high accuracy in several ethnic populations. Recurrent mutations and polymorphisms are those that occur in more than one family, such as at least two genetically distinct families. For example, a mutation or polymorphism that is only observed in a one family is not a recurrent mutation or polymorphism.

For example, the disclosed statistical analysis regarding concurrent testing of at least ten venous thrombosis associated genetic variations using the disclosed method demonstrated that the prediction of venous thrombosis is as accurate as at least 99% in Caucasians, at least 85% in Asians, and at least 88% in African populations. The disclosed methods, herein termed method evolved for recognition of thrombophilia (MERT), provide a rapid and cost-effective assay that allows for concurrent genetic testing in molecules statistically associated with venous thrombosis susceptibility, for example antithrombin II, protein C, protein S, fibrinogen, factor V, prothrombin (factor II), methylenetetrahydrofolate reductase (MTHFR), and angiotensin I-converting enzyme (ACE).

In one example, the method includes determining whether a subject has one or more mutations, polymorphisms, or both, in venous thrombosis-associated molecules that comprise, consist essentially of, or consist of, sequences from antithrombin III, protein C, protein S, fibrinogen, factor V, factor II, MTHFR and ACE. In one example, asymptomatic individuals are screened before or during their exposure to high risk situations that provoke thrombosis, such as pregnancy, puerperium, use of oral contraceptives or hormone replacement therapy, previous thrombosis history, prolonged immobilization, myeloproliferative disorders, malignancy, surgery, bone fracture, advanced age, antiphospholipid antibodies, or combinations thereof.

Although there are some already existing tests for screening up to six thrombophilia susceptibility single nucleotide polymorphisms, they have limited potential and a maximum predictive value of 1.7%. Such tests have a screening capacity that only allows them to detect single nucleotide polymorphisms (SNPs) that are prevalent only in Caucasian populations (Erali et al., Clin. Chem. 49:5, 2003; Evans et al., Clin. Chem 48:1406-11, 2002). In contrast, the methods and arrays disclosed herein are the first offering a highly accurate, overall venous thrombosis genetic susceptibility prediction, for example by screening mutations and polymorphisms (not only for SNPs but also for insertions and deletions) in those genes statistically associated with venous thrombosis. In particular examples, all known recurrent mutations and polymorphisms statistically associated with venous thrombosis are screened, or a subset of all such known mutations and polymorphisms. In some examples, a mutation or and polymorphism is statistically associated with venous thrombosis if it has a p-value of less than 0.005.

In particular examples, the method uses genomic DNA microarray technology to detect a subject's overall genetic susceptibility to venous thrombosis, and links the microarray data directly to the combined likelihood ratio for the panel of VT associated susceptibility genes applicable to diverse ethnic populations.

In a particular example, the method includes amplifying nucleic acid molecules obtained from a subject to obtain amplification products. The amplification products can comprise, consist essentially of, or consist of, sequences from antithrombin III, protein C, protein S, fibrinogen, factor V, prothrombin (factor I), methylenetetrahydrofolate reductase (MTHFR) and angiotensin I-converting enzyme (ACE) genes. The resulting amplification products are contacted with or applied to an array. The array includes oligonucleotide probes capable of hybridizing to antithrombin III, protein C, protein S, fibrinogen, factor V, factor II, MTHFR and ACE sequences that include one or more mutations, one or more polymorphisms, or combinations thereof. Examples of particular mutations and polymorphisms are provided in Table 1. In some examples, the array further includes oligonucleotides capable of hybridizing to wild-type antithrombin HI, wild-type protein C, wild-type protein S, wild-type fibrinogen, wild-type factor V, wild-type factor II, wild-type MTHFR and wild-type ACE. The amplification products are incubated with the array for a time sufficient to allow hybridization between the amplification products and oligonucleotide probes, thereby forming amplification products:oligonucleotide probe complexes. The amplification products:oligonucleotide probe complexes are then analyzed to determine if the amplification products include one or more mutations, polymorphisms, or both, in antithrombin III, protein C, protein S, fibrinogen, factor V, factor II, MTHFR or ACE. The presence of one or more mutations or one or more polymorphisms indicates that the subject has a genetic predisposition for venous thrombosis. In particular examples, the presence of more than one mutation or polymorphism indicates that the subject is at a greater risk for venous thrombosis than is a subject having only one mutation or polymorphism.

The disclosed method can accurately assess the overall genetic risk of developing venous thrombosis and thereby lead to avoiding venous thrombosis, for example by initiating appropriate prophylactic therapies in appropriate circumstances. The results presented herein demonstrate that concurrent use of a panel of genetic tests for at least eight molecules associated with venous thrombosis increases the positive predictive value more than 30 fold, when used for detecting venous thrombosis or a predisposition to its development. Therefore, methods of selecting venous thrombosis therapy are disclosed, which include detecting a mutation, a polymorphism, or combinations thereof (such as one or more substitutions, deletions or insertions) in at least one VT-related molecule of a subject, using the methods disclosed herein and if such mutation or polymorphism is identified, selecting a treatment to avoid venous thrombosis, delay the onset of venous thrombosis, or minimize its consequences.

Also disclosed are arrays capable of rapid, cost-effective multiple genetic testing for venous thrombosis genetic susceptibility, such as overall venous thrombosis genetic susceptibility. Such arrays include oligonucleotides that are complementary to antithrombin III, protein C, protein S, factor V, factor II, fibrinogen, MTHFR and ACE wild-type or mutated sequences, or both. Kits including such arrays for detecting a genetic predisposition to venous thrombosis in a subject are also disclosed.

The foregoing and other features and advantages of the disclosure will become more apparent from the following detailed description of a several embodiments.

### Sequencing Listing

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

ATIII

SEQ ID NO: 1 is an oligonucleotide sequence that can be used to probe for a wild-type antithrombin III sequence at nucleotide position 2770.

SEQ ID NO: 2 is an oligonucleotide sequence that can be used to probe for a 2770 insT in antithrombin Ill.

SEQ ID NO: 3 is an oligonucleotide sequence that can be used to probe for a wild-type antithrombin HI sequence at nucleotide positions 5311-5320.

SEQ ID NO: 4 is an oligonucleotide sequence that can be used to probe for a 5311-5320 del6bp in antithrombin III.

SEQ ID NO: 5 is an oligonucleotide sequence that can be used to probe for a wild-type antithrombin HI sequence at nucleotide positions 5356-5364.

SEQ ID NO: 6 is an oligonucleotide sequence that can be used to probe for a 5356-5364, delCTT in antithrombin III.

SEQ ID NO: 7 is an oligonucleotide sequence that can be used to probe for a wild-type antithrombin III sequence at nucleotide position 5381 C.

SEQ ID NO: 8 is an oligonucleotide sequence that can be used to probe for a 5381C/T replacement in antithrombin III.

SEQ ID NO: 9 is an oligonucleotide sequence that can be used to probe for a wild-type antithrombin III sequence at nucleotide position 5390 C.

SEQ ID NO: 10 is an oligonucleotide sequence that can be used to probe for a 5390 C/T replacement in antithrombin III.

SEQ ID NO: 11 is an oligonucleotide sequence that can be used to probe for a wild-type antithrombin III sequence at nucleotide position 5493 A.

SEQ ID NO: 12 is an oligonucleotide sequence that can be used to probe for a 5493 A/G replacement in antithrombin III.

SEQ ID NO: 13 is an oligonucleotide sequence that can be used to probe for a wild-type antithrombin III sequence at nucleotide position α(16)Arg: CGT6490 C.

SEQ ID NO: 14 is an oligonucleotide sequence that can be used to probe for a 6490 C/T replacement in antithrombin III.

SEQ ID NO: 15 is an oligonucleotide sequence that can be used to probe for a wild-type antithrombin III sequence at nucleotide position α(16)Arg: CGT9788 G.

SEQ ID NO: 16 is an oligonucleotide sequence that can be used to probe for a 9788 G/A replacement in antithrombin III.

SEQ ID NO: 17 is an oligonucleotide sequence that can be used to probe for a wild-type antithrombin III sequence at nucleotide position α(19)Arg: AGG9819 C.

SEQ ID NO: 18 is an oligonucleotide sequence that can be used to probe for a 9819 C/T replacement in antithrombin III.

SEQ ID NO: 19 is an oligonucleotide sequence that can be used to probe for a wild-type antithrombin III sequence at nucleotide position 13342.

SEQ ID NO: 20 is an oligonucleotide sequence that can be used to probe for a 13342 insA in antithrombin III.

SEQ ID NO: 21 is an oligonucleotide sequence that can be used to probe for a wild-type antithrombin III sequence at nucleotide position 13380 T.

SEQ ID NO: 22 is an oligonucleotide sequence that can be used to probe for a 13380 T/C replacement in antithrombin III.

SEQ ID NO: 23 is an oligonucleotide sequence that can be used to probe for a wild-type antithrombin III sequence at nucleotide position β(14)Arg, CGT6460 A.

SEQ ID NO: 24 is an oligonucleotide sequence that can be used to probe for a 6460 A/G replacement in antithrombin III.

SEQ ID NO: 25 is an oligonucleotide sequence that can be used to probe for a wild-type antithrombin III sequence at nucleotide position β(68)Ala, GCT13262 G.

SEQ ID NO: 26 is an oligonucleotide sequence that can be used to probe for a 13262 G/A replacement in antithrombin III.

SEQ ID NO: 27 is an oligonucleotide sequence that can be used to probe for a wild-type antithrombin III sequence at nucleotide position β(255)Arg, CGT13268 G.

SEQ ID NO: 28 is an oligonucleotide sequence that can be used to probe for a 13268 G/C replacement in antithrombin III.

SEQ ID NO: 29 is an oligonucleotide sequence that can be used to probe for a wild-type antithrombin III sequence at nucleotide position γ(275)Arg, CGC13268 G.

SEQ ID NO: 30 is an oligonucleotide sequence that can be used to probe for a 13268 G/T replacement in antithrombin III.

SEQ ID NO: 31 is an oligonucleotide sequence that can be used to probe for a wild-type antithrombin III sequence at nucleotide position γ(275)Arg, CG13295 C.

SEQ ID NO: 32 is an oligonucleotide sequence that can be used to probe for a 13295 C/T replacement in antithrombin III.

SEQ ID NO: 33 is an oligonucleotide sequence that can be used to probe for a wild-type antithrombin III sequence at nucleotide position γ(292)Gly, GGC 13296 G.

SEQ ID NO: 34 is an oligonucleotide sequence that can be used to probe for a 13296 G/A replacement in antithrombin III.

SEQ ID NO: 35 is an oligonucleotide sequence that can be used to probe for a wild-type antithrombin III sequence at nucleotide position γ(308)Asn, AAT13299 C.

SEQ ID NO: 36 is an oligonucleotide sequence that can be used to probe for a 13299 C/T replacement in antithrombin III.

SEQ ID NO: 37 is an oligonucleotide sequence that can be used to probe for a wild-type antithrombin III sequence at nucleotide position γ(318)Asp, GAC2484 T.

SEQ ID NO: 38 is an oligonucleotide sequence that can be used to probe for a γ(318)Asp/Gly: GAC/GGC2484 T/A replacement in antithrombin III.

SEQ ID NO: 39 is an oligonucleotide sequence that can be used to probe for a wild-type antithrombin III sequence at nucleotide position Thr312, ACT2586 C.

SEQ ID NO: 40 is an oligonucleotide sequence that can be used to probe for a 2586 C/T replacement in antithrombin III.

SEQ ID NO: 41 is an oligonucleotide sequence that can be used to probe for a wild-type antithrombin HI sequence at nucleotide position 2603 C.

SEQ ID NO: 42 is an oligonucleotide sequence that can be used to probe for a 2603 C/T replacement in antithrombin III.

SEQ ID NO: 43 is an oligonucleotide sequence that can be used to probe for a wild-type antithrombin III sequence at nucleotide position 2604 G.

SEQ ID NO: 44 is an oligonucleotide sequence that can be used to probe for a 2604 G/A replacement in antithrombin III.

SEQ ID NO: 45 is an oligonucleotide sequence that can be used to probe for a wild-type antithrombin III sequence at nucleotide position 2759 C.

SEQ ID NO: 46 is an oligonucleotide sequence that can be used to probe for a 2759 C/T replacement in antithrombin III.

SEQ ID NO: 47 is an oligonucleotide sequence that can be used to probe for a wild-type antithrombin m sequence at nucleotide position 5382 G.

SEQ ID NO: 48 is an oligonucleotide sequence that can be used to probe for a 5382 G/A replacement in antithrombin III.

SEQ ID NO: 49 is an oligonucleotide sequence that can be used to probe for a wild-type antithrombin III sequence at nucleotide position 13324 C.

SEQ ID NO: 50 is an oligonucleotide sequence that can be used to probe for a 13324 C/A replacement in antithrombin III.

SEQ ID NO: 51 is an oligonucleotide sequence that can be used to probe for a wild-type antithrombin III sequence at nucleotide position 13328 G.

SEQ ID NO: 52 is an oligonucleotide sequence that can be used to probe for a 13328 G/A replacement in antithrombin III.

SEQ ID NO: 53 is an oligonucleotide sequence that can be used to probe for a wild-type antithrombin III sequence at nucleotide position 13333 C.

SEQ ID NO: 54 is an oligonucleotide sequence that can be used to probe for a 13333 C/G replacement in antithrombin III.

SEQ ID NO: 55 is an oligonucleotide sequence that can be used to probe for a wild-type antithrombin HI sequence at nucleotide position 13337 C.

SEQ ID NO: 56 is an oligonucleotide sequence that can be used to probe for a 13337 C/A replacement in antithrombin III.

SEQ ID NO: 57 is an oligonucleotide sequence that can be used to probe for a wild-type antithrombin III sequence at nucleotide position 13338 C.

SEQ ID NO: 58 is an oligonucleotide sequence that can be used to probe for a 13338 C/T replacement in antithrombin III.

SEQ ID NO: 59 is an oligonucleotide sequence that can be used to probe for a wild-type antithrombin III sequence at nucleotide position 13392 G.

SEQ ID NO: 60 is an oligonucleotide sequence that can be used to probe for a 13392 G/C replacement in antithrombin III.

Protein C

SEQ ID NO: 61 is an oligonucleotide sequence that can be used to probe for a wild-type protein C sequence at nucleotide position 3363/3364 in protein C41G.

SEQ ID NO: 62 is an oligonucleotide sequence that can be used to probe for a 41 G/A replacement in protein C.

SEQ ID NO: 63 is an oligonucleotide sequence that can be used to probe for a wild-type protein C sequence at nucleotide position 1357C.

SEQ ID NO: 64 is an oligonucleotide sequence that can be used to probe for a 1357 C/T replacement in protein C.

SEQ ID NO: 65 is an oligonucleotide sequence that can be used to probe for a wild-type protein C sequence at nucleotide position 138 1C.

SEQ ID NO: 66 is an oligonucleotide sequence that can be used to probe for a 1381 C/T replacement in protein C.

SEQ ID NO: 67 is an oligonucleotide sequence that can be used to probe for a wild-type protein C sequence at nucleotide position 3103C.

SEQ ID NO: 68 is an oligonucleotide sequence that can be used to probe for a 3103 C/T replacement in protein C.

SEQ ID NO: 69 is an oligonucleotide sequence that can be used to probe for a wild-type protein C sequence at nucleotide position 3169 T.

SEQ ID NO: 70 is an oligonucleotide sequence that can be used to probe for a 3169 T/C replacement in protein C.

SEQ ID NO: 71 is an oligonucleotide sequence that can be used to probe for a wild-type protein C sequence at nucleotide position 3217 G.

SEQ ID NO: 72 is an oligonucleotide sequence that can be used to probe for a 3217 G/T replacement in protein C.

SEQ ID NO: 73 is an oligonucleotide sequence that can be used to probe for a wild-type protein C sequence at nucleotide position 3222 G.

SEQ ID NO: 74 is an oligonucleotide sequence that can be used to probe for a 3222 G/A replacement in protein C.

SEQ ID NO: 75 is an oligonucleotide sequence that can be used to probe for a wild-type protein C sequence at nucleotide position 3222 G.

SEQ ID NO: 76 is an oligonucleotide sequence that can be used to probe for a 3222 G/T replacement in protein C.

SEQ ID NO: 77 is an oligonucleotide sequence that can be used to probe for a wild-type protein C sequence at nucleotide position 3359G.

SEQ ID NO: 78 is an oligonucleotide sequence that can be used to probe for a 3359 G/A replacement in protein C.

SEQ ID NO: 79 is an oligonucleotide sequence that can be used to probe for a wild-type protein C sequence at nucleotide position 3360 C.

SEQ ID NO: 80 is an oligonucleotide sequence that can be used to probe for a 3360 C/A replacement in protein C.

SEQ ID NO: 81 is an oligonucleotide sequence that can be used to probe for a wild-type protein C sequence at nucleotide position 3363/3364 in protein C.

SEQ ID NO: 82 is an oligonucleotide sequence that can be used to probe for a 3363/4 insC in protein C.

SEQ ID NO: 83 is an oligonucleotide sequence that can be used to probe for a wild-type protein C sequence at nucleotide position 3438 C.

SEQ ID NO: 84 is an oligonucleotide sequence that can be used to probe for a 3438 C/T replacement in protein C.

SEQ ID NO: 85 is an oligonucleotide sequence that can be used to probe for a wild-type protein C sequence at nucleotide position 6128 T.

SEQ ID NO: 86 is an oligonucleotide sequence that can be used to probe for a 6128 T/C replacement in protein C.

SEQ ID NO: 87 is an oligonucleotide sequence that can be used to probe for a wild-type protein C sequence at nucleotide position 6152 C.

SEQ ID NO: 88 is an oligonucleotide sequence that can be used to probe for a 6152 C/T replacement in protein C.

SEQ ID NO: 89 is an oligonucleotide sequence that can be used to probe for a wild-type protein C sequence at nucleotide position 6182 C.

SEQ ID NO: 90 is an oligonucleotide sequence that can be used to probe for a 6182 C/T replacement in protein C.

SEQ ID NO: 91 is an oligonucleotide sequence that can be used to probe for a wild-type protein C sequence at nucleotide position 6216 C.

SEQ ID NO: 92 is an oligonucleotide sequence that can be used to probe for a 6216 C/T replacement in protein C.

SEQ ID NO: 93 is an oligonucleotide sequence that can be used to probe for a wild-type protein C sequence at nucleotide position 6245 C.

SEQ ID NO: 94 is an oligonucleotide sequence that can be used to probe for a 6245 C/T replacement in protein C.

SEQ ID NO: 95 is an oligonucleotide sequence that can be used to probe for a wild-type protein C sequence at nucleotide position 6246 G.

SEQ ID NO: 96 is an oligonucleotide sequence that can be used to probe for a 6246 G/A replacement in protein C.

SEQ ID NO: 97 is an oligonucleotide sequence that can be used to probe for a wild-type protein C sequence at nucleotide position 6265 G.

SEQ ID NO: 98 is an oligonucleotide sequence that can be used to probe for a 6265 G/C replacement in protein C.

SEQ ID NO: 99 is an oligonucleotide sequence that can be used to probe for a wild-type protein C sequence at nucleotide position 6274 C.

SEQ ID NO: 100 is an oligonucleotide sequence that can be used to probe for a 6274 C/T replacement in protein C.

SEQ ID NO: O11 is an oligonucleotide sequence that can be used to probe for a wild-type protein C sequence at nucleotide position 7176 G.

SEQ ID NO: 102 is an oligonucleotide sequence that can be used to probe for a 7176 G/A replacement in protein C.

SEQ ID NO: 103 is an oligonucleotide sequence that can be used to probe for a wild-type protein C sequence at nucleotide position 7253 C.

SEQ ID NO: 104 is an oligonucleotide sequence that can be used to probe for a 7253 C/T replacement in protein C.

SEQ ID NO: 105 is an oligonucleotide sequence that can be used to probe for a wild-type protein C sequence at nucleotide position 8403 C.

SEQ ID NO: 106 is an oligonucleotide sequence that can be used to probe for a 8403 C/T replacement in protein C.

SEQ ID NO: 107 is an oligonucleotide sequence that can be used to probe for a wild-type protein C sequence at nucleotide position 8481 A.

SEQ ID NO: 108 is an oligonucleotide sequence that can be used to probe for a 8481 A/G replacement in protein C.

SEQ ID NO: 109 is an oligonucleotide sequence that can be used to probe for a wild-type protein C sequence at nucleotide position 8485-7.

SEQ ID NO: 110 is an oligonucleotide sequence that can be used to probe for a 8485/6 delAC or 8486/7 delCA in protein C.

SEQ ID NO: 111 is an oligonucleotide sequence that can be used to probe for a wild-type protein C sequence at nucleotide position 8551 C.

SEQ ID NO: 112 is an oligonucleotide sequence that can be used to probe for a 8551 C/T replacement in protein C.

SEQ ID NO: 113 is an oligonucleotide sequence that can be used to probe for a wild-type protein C sequence at nucleotide position 8559 G.

SEQ ID NO: 114 is an oligonucleotide sequence that can be used to probe for a 8559 G/A replacement in protein C.

SEQ ID NO: 115 is an oligonucleotide sequence that can be used to probe for a wild-type protein C sequence at nucleotide position 8571 C.

SEQ ID NO: 116 is an oligonucleotide sequence that can be used to probe for a 8571 C/T replacement in protein C.

SEQ ID NO: 117 is an oligonucleotide sequence that can be used to probe for a wild-type protein C sequence at nucleotide position 8572 G.

SEQ ID NO: 118 is an oligonucleotide sequence that can be used to probe for a 8572 G/A replacement in protein C.

SEQ ID NO: 119 is an oligonucleotide sequence that can be used to probe for a wild-type protein C sequence at nucleotide position 8589 G.

SEQ ID NO: 120 is an oligonucleotide sequence that can be used to probe for a 8589 G/A replacement in protein C.

SEQ ID NO: 121 is an oligonucleotide sequence that can be used to probe for a wild-type protein C sequence at nucleotide position 8604 G.

SEQ ID NO: 122 is an oligonucleotide sequence that can be used to probe for a 8604 G/A replacement in protein C.

SEQ ID NO: 123 is an oligonucleotide sequence that can be used to probe for a wild-type protein C sequence at nucleotide position 8608 C.

SEQ ID NO: 124 is an oligonucleotide sequence that can be used to probe for a 8608 C/T replacement in protein C.

SEQ ID NO: 125 is an oligonucleotide sequence that can be used to probe for a wild-type protein C sequence at nucleotide position 8631 C.

SEQ ID NO: 126 is an oligonucleotide sequence that can be used to probe for a 8631 C/T replacement in protein C.

SEQ ID NO: 127 is an oligonucleotide sequence that can be used to probe for a wild-type protein C sequence at nucleotide position 8678-80.

SEQ ID NO: 128 is an oligonucleotide sequence that can be used to probe for a 8678-80 del3nt in protein C.

SEQ ID NO: 129 is an oligonucleotide sequence that can be used to probe for a wild-type protein C sequence at nucleotide position 8689 T.

SEQ ID NO: 130 is an oligonucleotide sequence that can be used to probe for a 8689 T/C replacement in protein C.

SEQ ID NO: 131 is an oligonucleotide sequence that can be used to probe for a wild-type protein C sequence at nucleotide position 8695 C.

SEQ ID NO: 132 is an oligonucleotide sequence that can be used to probe for a 8695 C/T replacement in protein C.

SEQ ID NO: 133 is an oligonucleotide sequence that can be used to probe for a wild-type protein C sequence at nucleotide position 84708763 G.

SEQ ID NO: 134 is an oligonucleotide sequence that can be used to probe for a 8763 G/A replacement in protein C.

SEQ ID NO: 135 is an oligonucleotide sequence that can be used to probe for a wild-type protein C sequence at nucleotide position 8857.

SEQ ID NO: 136 is an oligonucleotide sequence that can be used to probe for a 8857 delG in protein C.

SEQ ID NO: 137 is an oligonucleotide sequence that can be used to probe for a wild-type protein C sequence at nucleotide position 8895 A.

SEQ ID NO: 138 is an oligonucleotide sequence that can be used to probe for a 8895 A/C replacement in protein C.

SEQ ID NO: 139 is an oligonucleotide sequence that can be used to probe for a wild-type protein C sequence at nucleotide position 8924 C.

SEQ ID NO: 140 is an oligonucleotide sequence that can be used to probe for a 8924 C/G replacement in protein C.

SEQ ID NO: 141 is an oligonucleotide sequence that can be used to probe for a wild-type protein C sequence at nucleotide position 1387 C.

SEQ ID NO: 142 is an oligonucleotide sequence that can be used to probe for a 1387 C/T replacement in protein C.

SEQ ID NO: 143 is an oligonucleotide sequence that can be used to probe for a wild-type protein C sequence at nucleotide position 1388 G.

SEQ ID NO: 144 is an oligonucleotide sequence that can be used to probe for a 1388 G/A replacement in protein C.

SEQ ID NO: 145 is an oligonucleotide sequence that can be used to probe for a wild-type protein C sequence at nucleotide position 1432 C.

SEQ ID NO: 146 is an oligonucleotide sequence that can be used to probe for a 1432 C/T replacement in protein C.

SEQ ID NO: 147 is an oligonucleotide sequence that can be used to probe for a wild-type protein C sequence at nucleotide position −34 TG6218 C.

SEQ I) NO: 148 is an oligonucleotide sequence that can be used to probe for a −34, delG6218 C/T replacement in protein C.

SEQ ID NO: 149 is an oligonucleotide sequence that can be used to probe for a wild-type protein C sequence at −24 GTG (where 24 is the codonnucleotide position) 6219 G.

SEQ ID NO: 150 is an oligonucleotide sequence that can be used to probe for a 6219 G/A replacement in protein C.

SEQ ID NO: 151 is an oligonucleotide sequence that can be used to probe for a wild-type protein C sequence at nucleotide position 7219 C.

SEQ ID NO: 152 is an oligonucleotide sequence that can be used to probe for a 7219 C/A replacement in protein C.

SEQ ID NO: 153 is an oligonucleotide sequence that can be used to probe for a wild-type protein C sequence at nucleotide position 8470 G.

SEQ ID NO: 154 is an oligonucleotide sequence that can be used to probe for a 8470 G/A replacement in protein C.

SEQ ID NO: 155 is an oligonucleotide sequence that can be used to probe for a wild-type protein C sequence at nucleotide position 448744 G.

SEQ ID NO: 156 is an oligonucleotide sequence that can be used to probe for a 8744 G/A replacement in protein C.

SEQ ID NO: 157 is an oligonucleotide sequence that can be used to probe for a wild-type protein C sequence at nucleotide position 8769 C.

SEQ ID NO: 158 is an oligonucleotide sequence that can be used to probe for a 8769 C/T replacement in protein C.

SEQ ID NO: 159 is an oligonucleotide sequence that can be used to probe for a wild-type protein C sequence at nucleotide position 8790 G.

SEQ ID NO: 160 is an oligonucleotide sequence that can be used to probe for a 8790 G/A replacement in protein C.

SEQ ID NO: 161 is an oligonucleotide sequence that can be used to probe for a wild-type protein C sequence at nucleotide position 8886 G.

SEQ ID NO: 162 is an oligonucleotide sequence that can be used to probe for a 8886 G/A replacement in protein C.

SEQ ID NO: 163 is an oligonucleotide sequence that can be used to probe for a wild-type protein C sequence at nucleotide position −1654 C.

SEQ ID NO: 164 is an oligonucleotide sequence that can be used to probe for a—−1654 C/T replacement in protein C.

SEQ ID NO: 165 is an oligonucleotide sequence that can be used to probe for a wild-type protein C sequence at nucleotide position −1641 A.

SEQ ID NO: 166 is an oligonucleotide sequence that can be used to probe for a—−1641 A/G replacement in protein C.

SEQ ID NO: 167 is an oligonucleotide sequence that can be used to probe for a wild-type protein C sequence at nucleotide position −1476 A.

SEQ ID NO: 168 is an oligonucleotide sequence that can be used to probe for a—−1476 A/T replacement in protein C.

Protein S

SEQ ID NO: 169 is an oligonucleotide sequence that can be used to probe for a wild-type protein S sequence at position −34, TGC.

SEQ ID NO: 170 is an oligonucleotide sequence that can be used to probe for a −34, delG in protein S.

SEQ ID NO: 171 is an oligonucleotide sequence that can be used to probe for a wild-type protein S sequence at position −24, GTG.

SEQ ID NO: 172 is an oligonucleotide sequence that can be used to probe for a −24, GTG/GAG replacement in protein S.

SEQ ID NO: 173 is an oligonucleotide sequence that can be used to probe for a wild-type protein S sequence at position 19, GAA.

SEQ ID NO: 174 is an oligonucleotide sequence that can be used to probe for a 19, GAA/TAA replacement in protein S.

SEQ ID NO: 175 is an oligonucleotide sequence that can be used to probe for a wild-type protein S sequence at position 26, GAA.

SEQ ID NO: 176 is an oligonucleotide sequence that can be used to probe for a 26, GAA/GCA replacement in protein S.

SEQ ID NO: 177 is an oligonucleotide sequence that can be used to probe for a wild-type protein S sequence at position 44.

SEQ ID NO: 178 is an oligonucleotide sequence that can be used to probe for a 44, delCTTA in protein S.

SEQ ID NO: 179 is an oligonucleotide sequence that can be used to probe for a wild-type protein S sequence at position 46, GTT.

SEQ ID NO: 180 is an oligonucleotide sequence that can be used to probe for a 46, GTT/CTT replacement in protein S.

SEQ ID NO: 181 is an oligonucleotide sequence that can be used to probe for a wild-type protein S sequence at position intron d, exon 4, +1.

SEQ ID NO: 182 is an oligonucleotide sequence that can be used to probe for a intron d, G/A, exon 4, +1 replacement in protein S.

SEQ ID NO: 183 is an oligonucleotide sequence that can be used to probe for a wild-type protein S sequence at position 155, AAG.

SEQ ID NO: 184 is an oligonucleotide sequence that can be used to probe for a 155, AAG/GAG replacement in protein S.

SEQ ID NO: 185 is an oligonucleotide sequence that can be used to probe for a wild-type protein S sequence at position 217, AAT.

SEQ ID NO: 186 is an oligonucleotide sequence that can be used to probe for a 217, AAT/AGT replacement in protein S.

SEQ ID NO: 187 is an oligonucleotide sequence that can be used to probe for a wild-type protein S sequence at position 238, CAG.

SEQ ID NO: 188 is an oligonucleotide sequence that can be used to probe for a 238, CAG/TAG replacement in protein S.

SEQ ID NO: 189 is an oligonucleotide sequence that can be used to probe for a wild-type protein S sequence at position 265.

SEQ ID NO: 190 is an oligonucleotide sequence that can be used to probe for a 265, insT in protein S.

SEQ ID NO: 191 is an oligonucleotide sequence that can be used to probe for a wild-type protein S sequence at position 293, TCA.

SEQ ID NO: 192 is an oligonucleotide sequence that can be used to probe for a 293, TCA/TGA replacement in protein S.

SEQ ID NO: 193 is an oligonucleotide sequence that can be used to probe for a wild-type protein S sequence at position 295, GGC.

SEQ ID NO: 194 is an oligonucleotide sequence that can be used to probe for a 295, GGC/GTC replacement in protein S.

SEQ ID NO: 195 is an oligonucleotide sequence that can be used to probe for a wild-type protein S sequence at position intron j, exon 10, +5.

SEQ ID NO: 196 is an oligonucleotide sequence that can be used to probe for a intron j, G/A, exon 10, +5 replacement in protein S.

SEQ ID NO: 197 is an oligonucleotide sequence that can be used to probe for a wild-type protein S sequence at position 349, GAA.

SEQ ID NO: 198 is an oligonucleotide sequence that can be used to probe for a 349, GAA/AAA replacement in protein S.

SEQ ID NO: 199 is an oligonucleotide sequence that can be used to probe for a wild-type protein S sequence at position 372.

SEQ ID NO: 200 is an oligonucleotide sequence that can be used to probe for a 372, delCTTTTT, insAA in protein S.

SEQ ID NO: 201 is an oligonucleotide sequence that can be used to probe for a wild-type protein S sequence at position intron k, exon 12, −9.

SEQ ID NO: 202 is an oligonucleotide sequence that can be used to probe for a intron k, A/G, exon 12, −9 replacement in protein S.

SEQ ID NO: 203 is an oligonucleotide sequence that can be used to probe for a wild-type protein S sequence at position −25405, CTA.

SEQ ID NO: 204 is an oligonucleotide sequence that can be used to probe for a 405, CTA/CCA replacement in protein S.

SEQ ID NO: 205 is an oligonucleotide sequence that can be used to probe for a wild-type protein S sequence at position 410, CGA.

SEQ ID NO: 206 is an oligonucleotide sequence that can be used to probe for a 410, CGA/TGA replacement in protein S.

SEQ ID NO: 207 is an oligonucleotide sequence that can be used to probe for a wild-type protein S sequence at position 431.

SEQ ID NO: 208 is an oligonucleotide sequence that can be used to probe for a 431, insA in protein S.

SEQ ID NO: 209 is an oligonucleotide sequence that can be used to probe for a wild-type protein S sequence at position 465, TGG.

SEQ ID NO: 210 is an oligonucleotide sequence that can be used to probe for a 465, TGG/TGA replacement in protein S.

SEQ ID NO: 211 is an oligonucleotide sequence that can be used to probe for a wild-type protein S sequence at position 474, CGT.

SEQ ID NO: 212 is an oligonucleotide sequence that can be used to probe for a 474, CGT/TGT replacement in protein S.

SEQ ID NO: 213 is an oligonucleotide sequence that can be used to probe for a wild-type protein S sequence at position intron b, exon 2 +5522, CAG.

SEQ ID NO: 214 is an oligonucleotide sequence that can be used to probe for a 522, CAG/TAG replacement in protein S.

SEQ ID NO: 215 is an oligonuclebtide sequence that can be used to probe for a wild-type protein S sequence at position 534, CTG.

SEQ ID NO: 216 is an oligonucleotide sequence that can be used to probe for a 534, CTG/CGG replacement in protein S.

SEQ ID NO: 217 is an oligonucleotide sequence that can be used to probe for a wild-type protein S sequence at position intron k, exon 11 +54625, TGT.

SEQ ID NO: 218 is an oligonucleotide sequence that can be used to probe for a 625, TGT/CGT replacement in protein S.

SEQ ID NO: 219 is an oligonucleotide sequence that can be used to probe for a wild-type protein S sequence at position −2, CGT.

SEQ ID NO: 220 is an oligonucleotide sequence that can be used to probe for a −2, CGT/CTT replacement in protein S.

SEQ ID NO: 221 is an oligonucleotide sequence that can be used to probe for a wild-type protein S sequence at position 9, AAA.

SEQ ID NO: 222 is an oligonucleotide sequence that can be used to probe for a 9, AAA/GAA replacement in protein S.

SEQ ID NO: 223 is an oligonucleotide sequence that can be used to probe for a wild-type protein S sequence at position intron e, exon 5, +5.

SEQ ID NO: 224 is an oligonucleotide sequence that can be used to probe for a intron e, G/A, exon 5, +5 replacement in protein S.

SEQ ID NO: 225 is an oligonucleotide sequence that can be used to probe for a wild-type protein S sequence at position exon 15, 520 nt after the stop codon-25.

SEQ ID NO: 226 is an oligonucleotide sequence that can be used to probe for a −25, insT in protein S.

SEQ ID NO: 227 is an oligonucleotide sequence that can be used to probe for a wild-type protein S sequence at position 467, GTA.

SEQ ID NO: 228 is an oligonucleotide sequence that can be used to probe for a 467, GTA/GGA replacement in protein S.

SEQ ID NO: 229 is an oligonucleotide sequence that can be used to probe for a wild-type protein S sequence at position 633.

SEQ ID NO: 230 is an oligonucleotide sequence that can be used to probe for a 633, delAA in protein S.

SEQ ID NO: 231 is an oligonucleotide sequence that can be used to probe for a wild-type protein S sequence at position 636, TAA.

SEQ ID NO: 232 is an oligonucleotide sequence that can be used to probe for a 636, TAA/TAT replacement in protein S.

SEQ ID NO: 233 is an oligonucleotide sequence that can be used to probe for a wild-type protein S sequence at position intron k, exon 11 +54.

SEQ ID NO: 234 is an oligonucleotide sequence that can be used to probe for a intron k, C/T, exon 11 +54 replacement in protein S.

SEQ ID NO: 235 is an oligonucleotide sequence that can be used to probe for a wild-type protein S sequence at position 460, TCC.

SEQ ID NO: 236 is an oligonucleotide sequence that can be used to probe for a 460, TCC/CCC replacement in protein S.

SEQ ID NO: 237 is an oligonucle6tide sequence that can be used to probe for a wild-type protein S sequence at position 626, CCA.

SEQ ID NO: 238 is an oligonucleotide sequence that can be used to probe for a 626, CCA/CCG replacement in protein S.

SEQ ID NO: 239 is an oligonucleotide sequence that can be used to probe for a wild-type protein S sequence at position exon 15, 520 nt after the stop codon.

SEQ ID NO: 240 is an oligonucleotide sequence that can be used to probe for a exon 15, C/A 520 nt after the stop codon replacement in protein S.

Fibrinogen

SEQ ID NO: 241 is an oligonucleotide sequence that can be used to probe for a wild-type fibrinogen sequence at position a(16)Arg, CGT.

SEQ ID NO: 242 is an oligonucleotide sequence that can be used to probe for a α(16)Arg/Cys: CGT/TGT replacement in fibrinogen.

SEQ ID NO: 243 is an oligonucleotide sequence that can be used to probe for a wild-type fibrinogen sequence at position α(16)Arg, CGT.

SEQ ID NO: 244 is an oligonucleotide sequence that can be used to probe for a α(16)Arg/His: CGT/CAT replacement in fibrinogen.

SEQ ID NO: 245 is an oligonucleotide sequence that can be used to probe for a wild-type fibrinogen sequence at position α(19)Arg, AGG.

SEQ ID NO: 246 is an oligonucleotide sequence that can be used to probe for a α(19)Arg/Gly: AGG/GGG replacement in fibrinogen.

SEQ ID NO: 247 is an oligonucleotide sequence that can be used to probe for a wild-type fibrinogen sequence at position α(461)Lys, AAA.

SEQ ID NO: 248 is an oligonucleotide sequence that can be used to probe for a α(461)Lys/Stop: AAA/TAA replacement in fibrinogen.

SEQ ID NO: 249 is an oligonucleotide sequence that can be used to probe for a wild-type fibrinogen sequence at position α(554)Arg, CGT.

SEQ ID NO: 250 is an oligonucleotide sequence that can be used to probe for a α(554)Arg/Cys: CGT/TGT replacement in fibrinogen.

SEQ ID NO: 251 is an oligonucleotide sequence that can be used to probe for a wild-type fibrinogen sequence at position β(14)Arg, CGT.

SEQ ID NO: 252 is an oligonucleotide sequence that can be used to probe for a β(14)Arg/Cys: CGT/TGT replacement in fibrinogen.

SEQ ID NO: 253 is an oligonucle6tide sequence that can be used to probe for a wild-type fibrinogen sequence at position β(68)Ala, GCT.

SEQ ID NO: 254 is an oligonucleotide sequence that can be used to probe for a β(68)Ala/Thr: GCT/ACT replacement in fibrinogen.

SEQ ID NO: 255 is an oligonucleotide sequence that can be used to probe for a wild-type fibrinogen sequence at position β(255)Arg, CGT.

SEQ ID NO: 256 is an oligonucleotide sequence that can be used to probe for a β(255)Arg/Cys: CGT/TGT replacement in fibrinogen.

SEQ ID NO: 257 is an oligonucleotide sequence that can be used to probe for a wild-type fibrinogen sequence at position γ(275)Arg, CGC.

SEQ ID NO: 258 is an oligonucleotide sequence that can be used to probe for a γ(275)Arg/Cys: CGC/TGC replacement in fibrinogen.

SEQ ID NO: 259 is an oligonucleotide sequence that can be used to probe for a wild-type fibrinogen sequence at position γ(275)Arg, CGC.

SEQ ID NO: 260 is an oligonucleotide sequence that can be used to probe for a γ(275)Arg/His: CGC/CAC replacement in fibrinogen.

SEQ ID NO: 261 is an oligonucleotide sequence that can be used to probe for a wild-type fibrinogen sequence at position γ(292)Gly, GGC.

SEQ ID NO: 262 is an oligonucleotide sequence that can be used to probe for a γ(292)Gly/Val: GGC/GTC replacement in fibrinogen.

SEQ ID NO: 263 is an oligonucleotide sequence that can be used to probe for a wild-type fibrinogen sequence at position γ(308)Asn, AAT.

SEQ ID NO: 264 is an oligonucleotide sequence that can be used to probe for a γ(308)Asn/Lys: AAT/AAG replacement in fibrinogen.

SEQ ID NO: 265 is an oligonucleotide sequence that can be used to probe for a wild-type fibrinogen sequence at position γ(318)Asp, GAC.

SEQ ID NO: 266 is an oligonucleotide sequence that can be used to probe for a γ(318)Asp/Gly: GAC/GGC replacement in fibrinogen.

SEQ ID NO: 267 is an oligonucleotide sequence that can be used to probe for a wild-type fibrinogen sequence at position Thr312, ACT.

SEQ ID NO: 268 is an oligonucleotide sequence that can be used to probe for a Thr312Ala: ACT/GCT replacement in fibrinogen.

Factor V

SEQ ID NO: 269 is an oligonucleotide sequence that can be used to probe for a wild-type factor V sequence at nucleotide position 1691 G.

SEQ ID NO: 270 is an oligonucleotide sequence that can be used to probe for a 1691G/A replacement in factor V.

SEQ ID NO: 271 is an oligonucleotide sequence that can be used to probe for a wild-type factor V sequence at nucleotide position 1628G.

SEQ ID NO: 272 is an oligonucleotide sequence that can be used to probe for a 1628G/A replacement in factor V.

SEQ ID NO: 273 is an oligonucleotide sequence that can be used to probe for a wild-type factor V sequence at nucleotide position 4070A.

SEQ ID NO: 274 is an oligonucleotide sequence that can be used to probe for a 5382 G replacement in factor V.

SEQ ID NO: 275 is an oligonucleotide sequence that can be used to probe for a wild-type factor V sequence at nucleotide position 1090A.

SEQ ID NO: 276 is an oligonucleotide sequence that can be used to probe for a 1090A/G replacement in factor V.

SEQ ID NO: 277 is an oligonucleotide sequence that can be used to probe for a wild-type factor V sequence at nucleotide position 1091G.

SEQ ID NO: 278 is an oligonucleotide sequence that can be used to probe for a 1091 G/C replacement in factor V.

Factor II

SEQ ID NO: 279 is an oligonucleotide sequence that can be used to probe for a wild-type prothrombin sequence at nucleotide position 20210G.

SEQ ID NO: 280 is an oligonucleotide sequence that can be used to probe for a 20210G/A replacement in prothrombin.

MTHFR

SEQ ID NO: 281 is an oligonucleotide sequence that can be used to probe for a wild-type MTHFR sequence at nucleotide position 677 C.

SEQ ID NO: 282 is an oligonucleotide sequence that can be used to probe for a 677 C/T replacement in MTHFR.

SEQ ID NO: 283 is an oligonucleotide sequence that can be used to probe for a wild-type MTHFR sequence at nucleotide position 1298 A.

SEQ ID NO: 284 is an oligonucleotide sequence that can be used to probe for a 1298 A/C replacement in MTHFR.

ACE

SEQ ID NO: 285 is an oligonucleotide sequence that can be used to probe for the beginning portion of the wild-type ACE sequence with 288 bp insertion in intron 16.

SEQ ID NO: 286 is an oligonucleotide sequence that can be used to probe for the middle portion of the wild-type ACE sequence with 288 bp insertion in intron 16.

SEQ ID NO: 287 is an oligonucleotide sequence that can be used to probe for a mutant type ACE sequence with 288 bp deletion in intron 16.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. For example, the phrase “mutations or polymorphisms” or “one or more mutations or polymorphisms” means a mutation, a polymorphism, or combinations thereof, wherein “a” can refer to more than one.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

African: A human racial classification that includes persons having origins in any of the black racial groups of Africa. In some examples, includes dark-skinned persons who are natives or inhabitants of Africa, as well as persons of African descent, such as African-Americans, wherein such persons also retain substantial genetic similarity to natives or inhabitants of Africa. In a particular example, an African is at least 1/64 African.

Amplifying a nucleic acid molecule: To increase the number of copies of a nucleic acid molecule, such as a gene or fragment of a gene, for example a region of a venous thrombosis (VT)-associated gene. The resulting amplified products are called amplification products.

An example of in vitro amplification is the polymerase chain reaction (PCR), in which a biological sample obtained from a subject is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of ill vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Angiotensin I-converting enzyme (ACE): An enzyme that converts angiotensin I into the vasoconstrictor angiotensin II, and is involved in the degradation of bradykinin. Includes any ACE gene, cDNA, RNA, or protein from any organism, such as a human. Examples include the sequence disclosed in GenBank Accession No. BC048144 (as well as the corresponding genomic and protein sequence).

At least one variation in human ACE is associated with venous thrombosis: a polymorphism consisting of an insertion (ins) or deletion (del) of a 288-bp fragment in intron 16.

Anticoagulants: Agents that decrease or prevent abnormal blood clotting. Anticoagulants can avoid the formation of new clots, and prevent existing clots from growing (extending), for example by decreasing or stopping the production of proteins necessary for blood to clot. Examples include, but are not limited to, aspirin, heparin, and warfarin.

Antithrombin III (AT III): A member of the serpin (serine proteinase inhibitor) superfamily of proteins. AT III is the principal thrombin inhibitor, and has inhibitory effects on other coagulation factors, such as factors Ixa, Xa, XIa and XIIa. In addition, AT III accelerates the dissociation of the factor VIIa-tissue factor complex and prevents its rebinding. Includes the product of any AT III gene, cDNA, or RNA, or an AT III protein from any organism, such as a human. Examples include the mRNA sequence disclosed in GenBank Accession No. NM_{—}000488 (as well as the corresponding genomic and protein sequence). The gene coding for human AT III is localized on chromosome 1q23-25, spans 13.4 kb of DNA and has seven exons.

Heterozygous AT III deficiency is associated with increased risk for venous thrombosis. The molecular basis of AT III deficiency is highly heterogeneous. AT III deficiency is divided into type I (low plasma levels of both functional and immunological AT III) and type II (variant AT III in plasma). Type II is further subdivided into RS (defective reactive site), HBS (defective heparin-binding site) and PE (pleitropic, that is, multiple effects on function).

There are at least 127 distinct defects associated with AT III deficiency: 92 mutations for type I AT III deficiency (40 point mutations, 40 small insertions or deletions and 12 large deletions) and 35 mutations for type II AT III deficiency (12 RS, 12 HBS and 11 PE mutations, all point mutations). Among the type I mutations, at least 11 distinct mutations (7 point mutations and 4 deletions or insertions) have been described in multiple unrelated kindreds and the remaining mutations have been unique to single families which makes them individual mutations. In type II, 19 of the 35 mutations (seven RS, six HBS and six PE mutations) have been described in multiple unrelated kindreds and the remaining have been reported to be individual mutations.

Exemplary recurrent AT III gene mutations related to venous thrombosis are shown in Table 1.

Array: An arrangement of molecules, such as biological macromolecules (such as polypeptides or nucleic acids) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from a few (such as three) to at least 50, at least 100, at least 200, at least 250, at least 300, at least 500, at least 600, at least 1000, at least 10,000, or more. In particular examples, an array includes nucleic acid molecules, such as oligonucleotide sequences that are at least 15 nucleotides in length, such as about 15-40 nucleotides in length, such as at least 18 nucleotides in length, at least 21 nucleotides in length, or even at least 25 nucleotides in length. In one example, the molecule includes oligonucleotides attached to the array via their 5′- or 3′-end.

In particular examples, an array includes SEQ ID NOS: 1-287, or subsets thereof, such as odd-numbered SEQ ID NOS: 1-285 and SEQ ID NO: 286 (to detect wild-type VT-associated sequences), or even-numbered SEQ ID NOS: 2-284 and SEQ ID NO: 287 (to detect mutant or polymorphic VT-associated sequences), as well as at least 20 of the sequences shown in SEQ ID NOS: 1-287, such as at least 50, at least 75, at least 100, at least 150, at least 200, at least 250, or at least 260 of the sequences shown in SEQ ID NOS: 1-287.

Within an array, each arrayed sample is addressable, in that its location can be reliably and consistently determined within the at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Also contemplated herein are protein-based arrays, where the probe molecules are or include proteins, or where the target molecules are or include proteins, and arrays including nucleic acids to which proteins/peptides are bound, or vice versa.

Asian: A human racial classification that includes persons having origins in any of the original peoples of the Far East, Southeast Asia, the Indian subcontinent, or the Pacific Islands. This area includes, for example, China, India, Japan, Korea, the Philippine Islands, and Samoa. In particular examples, Asians include persons of Asian descent, such as Asian-Americans, that retain substantial genetic similarity to natives or inhabitants of Asia. In a particular example, an Asian is at least 1/64 Asian.

Binding or stable binding: An association between two substances or molecules, such as the hybridization of one nucleic acid molecule to another (or itself) and the association of an antibody with a peptide. An oligonucleotide molecule binds or stably binds to a target nucleic acid molecule if a sufficient amount of the oligonucleotide molecule forms base pairs or is hybridized to its target nucleic acid molecule, to permit detection of that binding. Binding can be detected by any procedure known to one skilled in the art, such as by physical or functional properties of the target:oligonucleotide complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both complementary strands.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Caucasian: A human racial classification traditionally distinguished by physical characteristics such as very light to brown skin pigmentation and straight to wavy or curly hair, which includes persons having origins in any of the original peoples of Europe, North Africa, or the Middle East. Popularly, the word “white” is used synonymously with “Caucasian” in North America. Such persons also retain substantial genetic similarity to natives or inhabitants of Europe, North Africa, or the Middle East. In a particular example, a Caucasian is at least 1/64 Caucasian. cDNA (complementary DNA): A piece of DNA lacking internal, non-coding segments (introns) and regulatory sequences which determine transcription. cDNA can be synthesized by reverse transcription from messenger RNA extracted from cells.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as antithrombin III, protein C, protein S, fibrinogen, factor V, factor II, MTHFR, and ACE) to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Deletion: The removal of one or more nucleotides from a nucleic acid sequence (or one or more amino acids from a protein sequence), the regions on either side of the removed sequence being joined together.

Factor V (FV): A protein that can act as a cofactor in the conversion of prothrombin to thrombin by factor Xa and (x-thrombin. Includes the product of any FV gene, cDNA, or RNA, or a FV protein from any organism, such as a human. Examples of FV nucleic acid sequences include the mRNA sequence disclosed in GenBank Accession No. NM_{—}000130 (as well as the corresponding genomic and protein sequence).

FV circulates in the plasma as a 330-kDa single chain glycoprotein. Downregulation of the procoagulant activity of activated FV (FVa) is accomplished by activated protein C (APC)-mediated proteolysis of FVa at three different sequential cleavage sites. Factor V is first cleaved at Arg 506, then at Arg 306, and finally at Arg 679. The cleavage of the peptide bond at Arg 506 is needed for the subsequent optimal exposure of cleavage sites at Arg 306 and Arg 679. Peptide bond cleavage at Arg 306 accounts for the initial 70% loss of activity and the subsequent cleavage at Arg 679 is responsible for the loss of the remaining activity.

At least five single nucleotide substitutions in the human FV gene are associated with increased thrombosis risk: 1691→G-A transition that results in a Arg506Gln polymorphism; 1628 G→A transition that results in a R485K polymorphism; 1091 G→C transition that results in a Arg3O6Thr mutation; 1090 A→G transition that results in a Arg306Gly mutation; and 4070 A→G transition that results in a His 1299Arg polymorphism.

Fibrinogen: A plasma protein with multiple functions in blood clotting, such as fibrin clot formation, factor XIII-mediated fibrin crosslinking, nonsubstrate thrombin binding, platelet aggregation, and fibrinolysis. Includes the product of any fibrinogen gene, cDNA, RNA, or a fibrinogen protein from any organism, such as a human. Examples of fibrinogen nucleic acid sequences include the mRNA sequences disclosed in GenBank Accession Nos. NM_{—}021871.1, BC007030, and NM_{—}021870 (for the α, β, and γ) subunits respectively, as well as the corresponding genomic and protein sequences).

Human fibrinogen is a 340-kDa glycoprotein, composed of two identical subunits linked through a disulfide bond. Each subunit includes three polypeptide chains (α, β, and γ), which are encoded by three separate genes on the long arm of human chromosome 4. Dysfibrinogenemia is caused by a variety of structural abnormalities in the fibrinogen molecule that result in abnormal fibrinogen function.

At least 25 single fibrinogen mutations (22 single nucleotide substitutions, one insertion and two deletions) are associated with increased thrombosis risk, and include the Thr312A1a polymorphism. At least thirteen mutations have been described in multiple reports from different unrelated kindreds and the remaining mutations have been unique to single families which makes them individual mutations.

Exemplary recurrent thrombophilic fibrinogen gene mutations and one common polymorphism related to venous thrombosis are shown in Table 1.

Genetic predisposition: Susceptibility of a subject to a genetic disease, such as venous thrombosis. However, such susceptibility may or may not result in actual development of the disease.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity

Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Detects Sequences that Share at Least 80% Identity
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Detects Sequences that Share at Least 50% Identity
Hybridization: 6×SSC at RT to 55° C. for 16-20 hours
Wash at least twice: 2×-3×SSC at RT to 55° C. for 20-30 minutes each.

Insertion: The addition of one or more nucleotides to a nucleic acid sequence, or the addition of one or more amino acids to a protein sequence.

Isolated: An “isolated” biological component (such as a nucleic acid molecule, protein, or organelle) has been substantially separated or purified away from other biological components in the cell of the organism in which the component naturally occurs, such as other chromosomal and extra-chromosomal DNA and RNA, proteins and organelles. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Label: An agent capable of detection, for example by ELISA, spectrophotometry, flow cytometry, or microscopy. For example, a label can be attached to a nucleic acid molecule, thereby permitting detection of the nucleic acid molecule. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Methylenetetrahydrofolate reductase (MTHFR): A protein that participates in the remethylation pathway of intracellular homocysteine metabolism. In the remethylation pathway catalyzed by methionine synthase, cobalamin acts as a cofactor and the methyl group is donated by 5-methyl-tetrahydrofolate, which derives from the reduction of 5, 10-methylenetetrahydrofolate by MTHFR. Includes the product of any MTHFR gene, cDNA, or RNA, or an MTHFR protein from any organism, such as a human. Examples include the mRNA sequence disclosed in GenBank Accession No. NM_{—}005957 (as well as the corresponding genomic and protein sequence).

The human MTHFR gene is located on chromosome 1p36.3, includes ˜17 kb of DNA and has 11 exons. At least two polymorphisms in human MTHRF are associated with venous thrombosis: a 677 C→T polymorphism and a 1298 A→C polymorphism.

Mutation: Any change of a nucleic acid sequence as a source of genetic variation. For example, mutations can occur within a gene or chromosome, including specific changes in non-coding regions of a chromosome, for instance changes in or near regulatory regions of genes. Types of mutations include, but are not limited to, base substitution point mutations (such as transitions or transversions), deletions, and insertions. Missense mutations are those that introduce a different amino acid into the sequence of the encoded protein; nonsense mutations are those that introduce a new stop codon; and silent mutations are those that introduce the same amino acid often with a base change in the third position of codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

Nucleic acid array: An arrangement of nucleic acid molecules (such as DNA or RNA) in assigned locations on a matrix, such as that found in cDNA arrays, or oligonucleotide arrays.

Nucleic acid molecules representing genes: Any nucleic acid molecule, for example DNA (intron or exon or both), cDNA or RNA, of any length suitable for use as a probe or other indicator molecule, and that is informative about the corresponding gene.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

The disclosure includes isolated nucleic acid molecules that include specified lengths of a VT-related nucleotide sequence. Such molecules can include at least 10, at least 15, at least 20, at least 21, at least 25, at least 30, at least 35, at least 40, at least 45 or at least 50 consecutive nucleotides of these sequences or more.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 bases, for example at least 8, at least 10, at least 15, at least 20, at least 21, at least 25, at least 30, at least 35, at least 40, at least 45, at least 50, at least 100 or even at least 200 bases long, or from about 6 to about 50 bases, for example about 10-25 bases, such as 12, 15, 20, 21, or 25 bases.

Oligonucleotide probe: A short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Open reading frame (ORF): A series of nucleotide triplets (codons) coding for amino acids without any internal termination codons. These sequences are usually translatable into a peptide.

Polymorphism: As a result of mutations, a gene sequence may differ among individuals. The differing sequences are referred to as alleles. The alleles that are present at a given locus (a gene's location on a chromosome is termed as a locus) are referred to as the individual's genotype. Some loci vary considerably among individuals. If a locus has two or more alleles whose frequencies each exceed 1% in a population, the locus is said to be polymorphic. The polymorphic site is termed a polymorphism. The term polymorphism also encompasses variations that produce gene products with altered function, that is, variants in the gene sequence that lead to gene products that are not functionally equivalent. This term also encompasses variations that produce no gene product, an inactive gene product, or increased or decreased activity gene product or even no biological effect.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

Primers: Short nucleic acid molecules, for instance DNA oligonucleotides 10-100 nucleotides in length, such as about 15, 20, 21, 25, 30 or 50 nucleotides or more in length. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Cailf., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length. Thus, for example, a primer including 30 consecutive nucleotides of a VT-related protein encoding nucleotide will anneal to a target sequence, such as another homolog of the designated VT-related protein, with a higher specificity than a corresponding primer of only 15 nucleotides. Thus, in order to obtain greater specificity, primers can be selected that includes at least 20, at least 21, at least 25, at least 30, at least 35, at least 40, at least 45, at least 50 or more consecutive nucleotides of a VT-related protein-encoding nucleotide sequences.

Protein C (PC): PC is activated after the binding of thrombin to its endothelial receptor, thrombomodulin. Activated PC inhibits clot formation by cleaving and inactivating factors Va and VIIIa. Includes the product of any PC gene, cDNA, or RNA, or a PC protein from any organism, such as a human. Examples include the mRNA sequence disclosed in GenBank Accession No. BC034377.1 (as well as the corresponding genomic and protein sequence).

The human PC gene is localized to human chromosome 2q13-14; it spans approximately 10 kb and contains nine exons. Loss-of-function mutations in the PC gene result in deficiency of PC, which is a well-established cause of VT. PC deficiency is classified into type I (low plasma concentrations of both functional and immunologic PC) and type II (low plasma levels of functional protein with normal antigen levels).

Among the at least 161 different PC gene mutations related to venous thrombosis in humans, at least 51 distinct mutations (48 point mutations, 2 deletions and 1 insertion) have been described in multiple unrelated kindreds and the remaining mutations are unique to single families which makes them individual mutations. At least forty recurrent mutations are associated with type I PC deficiency and 11 mutations have been found in patients with type II PC deficiency.

Three polymorphic sites (nt −1654C/T, −1641A/G and −1476A/T) located in the 5′ untranslated region of the PC gene also have an effect on plasma PC levels. Subjects carrying the CGT allele have lower plasma PC levels than subjects with the other genotypes and this allele is a risk factor for venous thrombosis.

Exemplary recurrent PC gene mutations and polymorphisms related to venous thrombosis are shown in Table 1.

Protein S (PS): A non-enzymatic cofactor for activated PC in the proteolytic inactivation of factors Va and VIIIa. Includes the product of any PS gene, cDNA, or RNA, or a PS protein from any organism, such as a human. Examples include the mRNA sequence disclosed in GenBank Accession No. NM_{—}000313.1 (as well as the corresponding genomic and protein sequence).

Human DNA contains two PS genes: the active PROS1 gene and the pseudogene PRSO2, which map to 3p11.1-q11.2. PRSO1 spans 80 kb genomic DNA and includes 15 exons and 14 introns. Loss-of-function mutations in PRSO1 lead to a deficiency of PS. Three types of PS deficiency are recognized based on plasma measurements: type I is characterized by low total and free PS antigen levels, type II by decreased activity and normal total and free PS antigen levels and type III by a selective reduction in free PS levels.

Among at least 131 different PS gene mutations related to venous thrombosis in humans, at least 32 distinct mutations (25 point mutations, 3 deletions, 3 insertions, and 1 deletion and insertion) have been described in multiple unrelated kindreds and the remaining mutations have been unique to single families which makes them individual mutations. Twenty-five recurrent mutations have been reported to be associated with quantitative (type I and/or type III) PS deficiency, 3 mutations with qualitative (type II) PS deficiency and type of the PS deficiency could not be determined in the remaining 4 mutations, either because one of the plasma assays was missing or because the subject was on oral anticoagulant therapy. Four recurrent polymorphisms in the PS gene cosegregate with the deficient phenotype in families with hereditary PS deficiency.

Exemplary recurrent PS gene mutations and polymorphisms related to venous thrombosis are shown in Table 1.

Prothrombin (Factor II, FII): The precursor of serine protease thrombin, which is a vitamin K-dependent glycoprotein. Activated by FXa (in the presence of FVa and phospholipids), FIIa exhibits procoagulant, anticoagulant, and antifibrinolytic activities. Includes the product of any FII gene, cDNA, or RNA, or a FII protein from any organism, such as a human. Examples include the mRNA sequence disclosed in GenBank Accession No. V00595.1 (as well as the corresponding genomic and protein sequence).

The human gene coding for FII is localized on chromosome 11, band 11p11-q12 and spans 21 kb of DNA. The FII gene is organized in 14 exons, separated by 13 introns, with 5′ and 3′-untranslated (UT) regions.

At least one single nucleotide substitution in the human FII gene is associated with increased thrombosis risk: G→ to A polymorphism at nucleotide 20210.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Sample: A biological specimen, such as those containing genomic DNA, RNA (including mRNA), protein, or combinations thereof. Examples include, but are not limited to, peripheral blood, urine, saliva, tissue biopsy, surgical specimen, amniocentesis samples, and autopsy material.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet etal., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol 215:403-10, 1990) is available from several sources, including the National Center for Biological Information (NCBI, National Library of Medicine, Building 38A, Room 8N805, Bethesda, MD 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. To compare two nucleic acid sequences, the options can be set as follows: −i is set to a file containing the first nucleic acid sequence to be compared (such as C:\seq1.txt); −j is set to a file containing the second nucleic acid sequence to be compared (such as C:\seq2.txt); −p is set to blastn; −o is set to any desired file name (such as C:\output.txt); −q is set to −1; −r is set to 2; and all other options are left at their default setting. For example, the following command can be used to generate an output file containing a comparison between two sequences: C:\B12seq −i c:\seq1.txt \j c:\seq2.txt −p blastn −o c:output.txt −q −1 −r 2.

To compare two amino acid sequences, the options of B12seq can be set as follows: −i is set to a file containing the first amino acid sequence to be compared (such as C:\seq1.txt); \j is set to a file containing the second amino acid sequence to be compared (such as C:\seq2.txt); −p is set to blastp; −o is set to any desired file name (such as C:\output.txt); and all other options are left at their default setting. For example, the following command can be used to generate an output file containing a comparison between two amino acid sequences: C:\B12seq −i c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (i.e., 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

| | |
|---|---|
| | 1 20 |
| Target Sequence: | AGGTCGTGTACTGTCAGTCA |
| | \| \|\| \|\|\| \|\|\|\| \|\|\|\| \| |
| Identified Sequence: | ACGTGGTGAACTGCCAGTGA |

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, at least 80%, at least 85%, at least 90%, at least 95%, at least 98%, or at least 99% sequence identity.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 60%, at least 70%, at least 75%, at least 80%, at least 85%, at least 90%, at least 95%, at least 98%, or at least 99% sequence identity. When less than the entire sequence is being compared for sequence identity, homologs will typically possess at least 75% sequence identity over short windows of 10-20 amino acids, and can possess sequence identities of at least 85%, at least 90%, at least 95% or at least 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, at least 70%, at least 80%, at least 90%, at least 95%, at least 98%, or at least 99% sequence identity determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Single nucleotide polymorphism (SNP): A single base (nucleotide) difference in a DNA sequence among individuals in a population. SNPs can be causative (actually involved in or influencing the condition or trait to which the SNP is linked) or associative (linked to but not having any direct involvement in or influence on the condition or trait to which the SNP is linked).

Subject: Living multi-cellular vertebrate organisms, a category that includes human and non-human mammals (such as veterinary subjects).

Target sequence: A sequence of nucleotides located in a particular region in a genome (such as a human genome or the genome of any mammal) that corresponds to one or more specific genetic abnormalities, such as one or more nucleotide substitutions, deletions, insertions, amplifications, or combinations thereof. The target can be for instance a coding sequence; it can also be the non-coding strand that corresponds to a coding sequence. Examples of target sequences include those sequences associated with venous thrombosis, such as those listed in Table 1.

Venous thrombosis (VT): A blood clot that forms within a vein. In particular examples, VT is associated with sluggish blood flow (for example as occurs during prolonged bed rest, pregnancy, and surgery) or with rapid coagulation of the blood. Examples include deep venous thromboses (DVTs) that form in the deep veins of the legs or in the pelvic veins. Such thrombi sometimes migrate to the lungs and form pulmonary emboli that lead to cardiopulmonary collapse and death.

Venous thrombosis (VT)-related (or associated) molecule: A molecule that is involved in the development of venous thrombosis. Such molecules include, for instance, nucleic acids (such as DNA, cDNA, or mRNAs) and proteins. Specific examples of VT-related molecules include those listed in Table 1, as well as fragments of the full-length genes or cDNAs that include the mutation(s), polymorphism(s), or both, responsible for increasing an individual's susceptibility to VT, and proteins and protein fragments encoded thereby.

VT-related molecules can be involved in or influenced by venous thrombosis in many different ways, including causative (in that a change in a VT-related molecule leads to development of or progression to venous thrombosis) or resultive (in that development of or progression to venous thrombosis causes or results in a change in the VT-related molecule).

Wild-type: A genotype that predominates in a natural population of organisms, in contrast to that of mutant forms.

Mutations and Polymorphisms Involved in Venous Thrombosis

Complex traits such as venous thrombosis can be understood by assuming an interaction between different mutations, polymorphisms, or both, in candidate susceptibility genes. The risk that is associated with each genetic defect may be relatively low in isolation but the simultaneous presence of several mutations or polymorphisms may dramatically increase disease susceptibility. Moreover, environmental factors can interact with one or more genetic variations to add further to the risk. Expression of a venous thrombosis phenotype is dependent on the interaction of gene products from several loci and environmental or acquired influences. Therefore, VT is a complex genetic disorder.

Several mutations and polymorphisms (such as one or more nucleotide substitutions, insertions, deletions, or combinations thereof) in genes associated with a risk of developing VT are known. However, a combination of mutations and polymorphisms (such as in genes statistically associated with VT) that permit accurate prediction of a subject's overall genetic predisposition to VT, in multiple ethnic groups, has not been previously identified.

Protein C, Protein S, and Antitdzrombihi III

Several genes involved in venous thrombosis, including protein C (PC), protein S (PS), and antithrombin III, are involved in anticoagulant pathways. PC and PS deficiencies result in defects in the activated PC anticoagulant system.

At least 161 different detrimental PC gene mutations have been reported in humans (Reitsma et al., Thromb. Haemost. 73:876-89, 1995). Among these 161 different PC gene mutations, only 51 distinct mutations (48 point mutations, 2 deletions and 1 insertion) have been described in multiple unrelated kindreds and the remaining 109 mutations have been unique to single families which makes them individual mutations. Forty recurrent mutations are associated with type I PC deficiency and 11 mutations were observed in patients with type II PC deficiency. Three polymorphic sites (nt −1654C/T, −1641A/G and −1476A/T) are located in the 5′-untranslated region of the gene that have an effect on plasma PC levels.

PS deficiency has a highly heterogeneous molecular basis with at least 131 different mutations (Gandrille et al., Thromb. Haemost. 84:918, 2000). Among all PS gene detrimental mutations, only 32 distinct mutations (25 point mutations, 3 deletions, 3 insertions, and 1 deletion and insertion) have been described in multiple unrelated kindreds and the remaining 100 mutations have been unique to single families which make them individual mutations. Twenty-five recurrent mutations have been reported to be associated with quantitative (type I and/or type II) PS deficiency, and 3 mutations with qualitative (type II) PS deficiency. Four recurrent polymorphisms in the PS gene cosegregate with the deficient phenotype in families with hereditary PS deficiency.

The prevalence of PC deficiency in the general population is approximately 1/300. The carrier state for PC and PS deficiencies is associated with approximately a 10-fold increased thrombosis risk for VT. Homozygous PC and PS deficiency is usually associated with a severe clinical phenotype known as purpura fulminans, characterized by extensive thromboses in the microcirculation early after birth.

Heterozygous antithrombin III (AT III) deficiency is associated with increased risk for VT. There are at least 127 distinct defects (Lane et al., Thromb. Haemost. 77:197-211, 1997) associated with AT III deficiency: 92 mutations for type I AT III deficiency (40 point mutations, 40 small insertions or deletions and 12 large deletions) and 35 mutations for type II AT III deficiency (12 RS, 12 HBS and 11 PE mutations, all point mutations). Among the type I mutations, only 11 distinct mutations (7 point mutations and 4 deletions or insertions) have been described in multiple unrelated kindreds and the remaining 81 mutations have been unique to single families which makes them individual mutations. In type II, 19 of the 35 mutations (seven RS, six HBS and six PE mutations) have been described in multiple unrelated kindreds and the remaining 16 have been reported to be individual mutations.

The prevalence of AT III deficiency in the general population ranges from 0.2/1000 to 18/1000. In a population-based control study, a five-fold increased risk for VT linked AT III deficiency was reported. The prevalence of AT m deficiency in thrombosis patients ranges from 1% to 8%.

Factor V, Prothrombin, and Fibribiogeni

Other genes involved in venous thrombosis include Factor V (FV), prothrombin (Factor II), and fibrinogen, which are involved in procoagulant pathways. Altered activity of mutated FV is the most common hereditary blood coagulation disorder that affects development of VT (Nicolaes et al., Arterioscler. Thromb. Vasc. Biol. 22:530-8, 2002). Downregulation of the procoagulant activity of activated FV (FVa) is accomplished by activated protein C (APC)-mediated proteolysis of FVa at three different sequential cleavage sites: Arg 506, Arg 306, and Arg 679 (the numbering of nucleotides or amino acids herein refer to human genes). A defect at one or more of these three cleavage sites can affect the APC inactivation process even though procoagulation activity may remain normal.

At least five recurrent single nucleotide substitutions in the human FV gene are associated with increased thrombosis risk. In 90% of cases, resistance to APC due to a single nucleotide substitution (FV Leiden; 1691GSA) that results in the replacement of Arg506 with Gln (R506Q). Its prevalence in Caucasian populations is approximately 5% and is as high as 20% to 40% in patients with VT. However, very few cases of FV Leiden have been reported among other races and it has not been found in South East Asia and Africa, so it is believed that FV Leiden mutation is specific to Caucasians (Takamiya et al., Thromb. Haemost. 74:996, 1995; Fujimura et al., Thromb. Haemost.74:1381-2, 1995; Chan et al., Thromb. Haemost. 75:522-3, 1996).

Another single nucleotide substitution in the FV gene, R485K, is associated with increased thrombosis risk in Far East populations (Hiyoshi et al., Thromb. Haemost. 80:705-6, 1998; Le et al., Clin. Genet. 57:296-303, 2000). The R485K polymorphism is a G→A transition occurring at nucleotide 1628 and results in the replacement of the codon AGA of Arg 485 by an AAA codon predicting a Lys residue. Although the frequency of the K485 allele is low in Caucasians and high in Asians and high in Asians and Africans, this polymorphism was shown to be associated with increased thrombosis risk in both Far East and Caucasian populations (Faisel et al., Eur. J. Hum. Genet. 12:187-91, 2004)

Three other single nucleotide substitutions are associated with increased risk of thrombosis in different populations. Two mutations in exon 7 of the human FV gene affect the Arg306 APC cleavage site. These two mutations also have a heterogeneous racial distribution. The FV Cambridge mutation is a G to C transition at nucleotide position 1091 and predicts replacement of arginine with a threonine at amino acid position 306 (Arg306Thr). This mutation has only been described in Caucasian populations (Franco et al., Thromb. Haemost. 81:312-3,1999). Thesecondmutation, FV Hong Kong, is an A to G transition at nucleotide position 1090 and changes Arg306 to Gly. Although this mutation was originally described in Chinese populations, it has a prevalence of 0.4% in Caucasians (Franco et al., Thromb. Haemost. 81:312-3, 1999).

Another single nucleotide substitution in exon 13 of the human FV gene, referred to as the R2 allele, is an A to G transition at nucleotide position 4070, which replaces His by Arg at position 1299 (H1299R). The prevalence of R2 allele is significantly higher in the patients with VT than in the healthy controls, with respective values of 18.5% and 11.4% (Alhenc-Gelas et al., Thromb. Haemost. 81:193-7, 1999). This polymorphism has a prevalence of 11.9% in U.S. Caucasians, 5.6% in African-Americans, 13.4% in Asian or Pacific Islanders and 11.3% in Hispanics (Benson et al., Thromb. Haemost. 86:1188-92, 2001).

At least one single nucleotide substitution is associated with increased thrombosis risk in the protbrombin (Factor II, FII) gene. The G→ to A polymorphism at nucleotide 20210 in the 3′-UT region of the prothrombin gene is the second most frequently inherited risk factor for venous thrombosis (Poort et al., Blood 88:3698-703, 1996). FII G20210A is associated with hyperprothrombinemia and a two- to five fold increased risk of VT. It is found in 1%-3% of subjects in healthy subjects and in 6%-18% of patients with VT in Caucasian populations (Rosendaal et al., Thromb. Haemost. 79:706-8, 1998), but is quite rare in African Americans and Amerindians from Brazil (Dilley et al., Blood 90:652a, 1997; Arruda et al., Thromb. Haemost. 78:1430-3, 1997). This mutation has not been found in West African, Amazonian Indian, Australasian, Latin American, Japanese or Chinese subjects (Ferraresi et al., Arterioscler. Thrornb. Vasc. Biol 17:2418-22, 1997; Rahimy et al., Thromb. Haemost. 79:444-5, 1998; Isshiki et al., Blood Coagul. Fibrinol. 9:105-6, 1998; Miyata et al., Blood Coagul. Fibrinol. 9:451-2, 1998, Rees et al. Br. J. Haematol. 105:564-566, 1999).

At least 25 thrombophilic fibrinogen mutations (22 single nucleotide substitutions, 1 insertion and two deletions) are associated with VT (De Stefano et al., Br. J. Haematol. 106:564-8, 1999). At least thirteen of the mutations are from different unrelated kindreds; the remaining mutations are unique to single families, making them individual mutations. The prevalence of inherited dysfibrinogenemia among the general population is unknown; however, the prevalence among patients with a history of venous thrombosis is 0.8 % (Carter et al., Blood 96:1177-9, 2000). A common polymorphism leading to a substitution of threouine by alanine at codon 312 (Thr312Ala polymorphism) within the carboxy-terminal end of the fibrinogen Aα chain is associated with venous thromboembolism via influencing clot stability and predisposing clots to embolization in the venous vascular trees (Carter et al., Blood 96:1177-9, 2000; Standeven et al., Circulation 107:2326-30, 2003; Hayes, Arch. Pathol Lab. Med. 126:1387-90, 2002). The polymorphism is observed in 51% of patients with pulmonary embolism and in 40% of healthy subjects. No differences have been found in genotype distribution for Thr312Ala polymorphism in Caucasians and Asians and this polymorphism is associated with elevated fibrinogen levels in both populations (Liu et al., J. Med. Genet. 38:31-5, 2001; Kain et al., Am. J. Epidemiol. 156:174-9, 2002).

Aytgiotensit I-Converting Enzyme and Metliylenetetralhydrofolate Reductase

Additional genes involved in venous thrombosis include, but are not limited to, angiotensin I-converting enzyme (ACE) and methylenetetrahydrofolate reductase (MTHFR).

The renin angiotensin system affects hemostasis through different mechanisms. In intron 16 of the human ACE gene, a polymorphism consisting of an insertion or deletion of a 288-bp fragment is known (Rigat et al., Nuc. Acids Res. 20:1433, 1992). The ACE DD genotype is associated with increased levels of circulating enzyme and 3 to 10-fold increased risk to venous thromboembolism among Caucasians and African-Americans. The ACE DD genotype has also been reported in the Japanese population.

Mild-to-moderate hyperhomocysteinemia (fasting levels of total homocysteine between 15 and 100 μmol/) is an established risk factor for VT and is associated with two- to four fold increased risk of thrombosis. Although it can be caused by several acquired causes including nutritional deficiencies of vitamin B12, vitamin B6 and folate, advanced age, chronic renal failure and the use of anti-folic drugs, two common polymorphisms in the methylenetetrahydrofolate reductase (MTHFR) gene are associated with mild-to moderate hyperhomocysteinemia (Cattaneo, Thromb. Haemost. 81:165-76, 1999; Franco and Reitsma, Hum. Genet. 109:369-84, 2001).

MTHFR 677 C→T polymorphism is located in human exon 4 at the folate binding site, converting an alanine into a valine. In its homozygous state, C677T polymorphism is associated with thermolability of MTHFR, leading to 60-70% reduction of the enzymatic activity and mild to moderate hyperhomocysteinemia (Franco and Reitsma, Hum. Genet. 109:369-84, 2001; Frosst et al., Nat. Genet. 10:111-3, 1995). The C677T polymorphism in human MTHFR has a relatively high frequency throughout the world, TT genotype is present in about 5% to 17% of the general population with a very heterogeneous distribution among different ethnic groups, highest prevalence in Europe and lowest prevalence in Africa (Frosst et al., Nat. Genet. 10:111-3, 1995; Schneider et al., Am. J. Hum. Genet. 62:1258-60, 1998; De Franchis et al., Am. J. Hum. Genet. 59:262-4, 1996; Ma et al., Circulation 94:2410-6, 1996; Deloughery et al., Circulation 94:3074-8, 1996; Arruda V et al., Thronmb. Haemost. 77:818-21, 1997). Homozygous MTHRF C677T polymorphism is an independent risk factor for venous thrombosis with a prevalence of 11%-27% in venous thrombosis patients in Caucasians but not associated with VT in Asians and Africans (Arruda et al., Thromb. Haemost. 77:818-21, 1997; Margaglione et al., Thromb. Haemost. 79:907-11, 1998; Salomon et al., Arterioscler. Thromb. Vasc. Biol. 19:511-8, 1999).

Another MTHFR polymorphism, 1298 A→C, is in human exon 7 within the presumptive regulatory domain and converts a glutamine into an alanine. By itself, this polymorphism does not appear to be associated with hyperhomocysteinemia but compound heterozygosity with MTHFR 677 C→T results in decreased enzyme activity and increased homocysteine levels (Weisberg et al., Mol. Genet. Metab. 68:511-2, 1999).

### Determining Genetic Predisposition to Venous Thrombosis

Provided herein are methods of determining whether a subject, such as an otherwise healthy subject, or a subject suspected or at risk of developing thrombi, is susceptible to developing venous thrombosis (VT). The methods involve detecting an abnormality (such as a mutation or polymorphism) in at least one VT-related molecule of the subject, such as a nucleic acid molecule that encodes a coagulation-related protein. Specific encompassed embodiments include diagnostic or prognostic methods in which one or more mutations or polymorphisms in a VT-related nucleic acid molecule in cells of the individual is detected. In particular embodiments, an abnormality is detected in a subset of VT-related molecules (such as nucleic acid sequences), or all known VT-related molecules, that selectively detect a genetic predisposition of a subject to develop VT.

In particular examples, the subset of molecules includes a set of 10 VT-related susceptibility alleles associated with venous thrombotic events, wherein the 10 VT-related susceptibility alleles are present in at least 95% of Caucasians subjects who are at risk for (or who have experienced) a venous thrombosis. In particular examples, the 10 VT-related susceptibility alleles are present in at least 98% of Caucasians, such as at least 99%, and at least 82% of Asians and African populations, such as at least 85% of Africans who have or are at risk of developing a VT.

In yet other examples, the number of VT-related susceptibility alleles screened is at least 10, for example at least 15, at least 20, at least 50, at least 100, at least 143, at least 200, at least 287, or even at least 500 alleles. In other examples, the methods employ screening no more than 600, no more than 500, no more than 400, no more than 287, no more than 200, no more than 143, no more than 100, no more than 50, or no more than 10 VT-related susceptibility alleles. Examples of particular VT-related susceptibility alleles are shown in Table 1.

As used herein, the term “VT-related molecule” includes VT-related nucleic acid molecules (such as DNA, RNA or cDNA) and VT-related proteins. The term is not limited to those molecules listed in Table 1 (and molecules that correspond to those listed), but also includes other nucleic acid molecules and proteins that are influenced (such as to level, activity, localization) by or during venous thrombosis, including all of such molecules listed herein.

Examples of VT-related genes include antithrombin III, protein C, protein S, fibrinogen, factor V, prothrombin (factor ID), methylenetetrahydroflate reductase (MTHFR) and angiotensin-I converting enzyme (ACE). In certain examples, abnormalities are detected in at least one VT-related nucleic acid, for instance in at least 2, at least 3, at least 4, at least 5, at least 6, at least 7, at least 8, at least 10, at least 15 or more VT-related nucleic acid molecules. In particular examples, certain of the described methods employ screening no more than 100, no more than 50, no more than 40, no more than 30, no more than 20, or no more than 15 VT-related genes.

This disclosed method (MERT) provides a rapid, straightforward, accurate and affordable multiple genetic screening method for screening in one assay overall inherited venous thrombosis susceptibility, that has a high predictive power for identification of asymptomatic carriers. It allows early recognition of subjects who may require prophylactic anticoagulant therapy during high risk situations, such as pregnancy, puerperium, use of oral contraceptives or hormone replacement therapy, trauma, surgery, fractures, prolonged immobilization, long air journeys (such as those more than 4 hours), advanced age, antiphospholipid antibodies, previous thrombosis history, myeloproliferative disorders, malignancy, or combinations thereof. The disclosed assay can be used to reduce the yearly incidence of venous thrombosis by early identification of individuals at inherited risk. By detecting individuals before they develop symptoms, effective preventive measures, such as early thromboprophylaxis or even decisions such as avoiding the use of oral contraceptives or hormone replacement therapy, can be instituted.

As discussed above, there are differences in the causes of the inherited venous thrombosis among different ethnic groups. Whereas FV Leiden and prothrombin G0210A polymorphisms are the most prevalent risk factors for venous thrombosis in Caucasians, Asian and African patients exhibit no or very rare FV Leiden or prothrombin G20210A polymorphisms. The disclosed methods and arrays are designed to determine inherited venous thrombophilia risk not only in Caucasians but also diverse ethnic populations. In one particular example, the method has a high predictive power in different ethnic populations (such as at least 98% for Caucasians, at least 84% for Asians and at least 87% for Africans). In other examples, the method detects abnormalities in VT-related molecules (such as nucleic acid sequences) wherein the abnormalities are found in at least 99% of Caucasians, at least 85% of Asians, and at least 88% of Africans who have had a VT. Therefore, the applicability of the disclosed methods and arrays in diverse ethnic populations makes it a powerful approach.

In particular examples, the disclosed methods and arrays are cost-effective compared to the currently available plasma-based thrombophilia screening panel, which includes antigenic and activity based determination of protein C and S, AT III antigen and activity, thrombin time and reptilase time for dysfibrinogenernia, quantitative determination of fibrinogen level and PCR-based direct mutation analysis of FV Leiden, prothrombin 20210A and MTHFR polymorphisms.

For example, the disclosed method provides advantages over AT III deficiency assays, because the disclosed method detects type I and II deficiencies in one assay instead of two subsequent tests, and detects variant AT III type II defects that can unfortunately be missed due to long incubation times of many automated functional heparin cofactor assay analyzers used in clinical practice, and by avoiding high rates of false positivity.

In other examples, the disclosed method provides advantages over PC deficiency assays, including the clotting assay of functional PC level, immunological PC assay and chromogenic assay of PC activity, because the disclosed method can in one embodiment detect type I and II deficiencies in one assay instead of three subsequent tests and overcomes the difficulty of distinguishing healthy subjects from asymptomatic PC deficient individuals due to the presence of a significant overlap between low normal levels and mild PC deficiency, by avoiding the under-determination of PC deficient individuals because of the increase in PC concentration as a function of age which is approximately 4% per decade, and by avoiding high rates of false positivity.

In other examples, the disclosed method provides advantages over PS deficiency assays, which include clotting assay of functional PS level, immunoassay of PS, and enzyme-linked immunosorbent assays for total and free PS measurements because the disclosed method can in one embodiment detect quantitative and functional defects in one assay instead of four subsequent phenotypic assays and overcomes the difficulty in the diagnosis of PS deficiency with the immunologic assay which is complicated by the presence of two molecular forms of PS in the plasma (free PS and C4b-BP/PS complexes), overcomes the difficulty of distinguishing healthy subjects from asymptomatic PS deficient individuals due to the presence of overlapping values between controls and PS deficient individuals, especially those with type III PS deficiency, and by avoiding high rates of false positivity.

In other examples, the disclosed method provides an advantage over assays for dysfibrinogenemia which include thrombin time and reptilase time as first line tests, because the disclosed method avoids high rates of false positivity.

Clinical Specimens

Appropriate specimens for use with the current disclosure in determining a subject's genetic predisposition to VT include any conventional clinical samples, for instance blood or blood-fractions (such as serum). Techniques for acquisition of such samples are well known in the art (for example see Schluger et al. J. Exp. Med. 176:1327-33, 1992, for the collection of serum samples). Serum or other blood fractions can be prepared in the conventional manner. For example, about 200 [L of serum can be used for the extraction of DNA for use in amplification reactions.

Once a sample has been obtained, the sample can be used directly, concentrated (for example by centrifugation or filtration), purified, or combinations thereof, and an amplification reaction performed. For example, rapid DNA preparation can be performed using a commercially available kit (such as the InstaGene Matrix, BioRad, Hercules, Cailf.; the NucliSens isolation kit, Organon Teknika, Netherlands). In one example, the DNA preparation method yields a nucleotide preparation that is accessible to, and amenable to, nucleic acid amplification.

Amplification of Nucleic Acid Molecules

The nucleic acid samples obtained from the subject to obtain amplification products, including sequences from AT III, protein C, protein S, fibrinogen, factor V, prothrombin (factor II), MTHFR, and ACE can be amplified from the clinical sample prior to detection. In one example, DNA sequences are amplified. In another example, RNA sequences are amplified.

Any nucleic acid amplification method can be used. In one specific, non-limiting example, polymerase chain reaction (PCR) is used to amplify the nucleic acid sequences associated with venous thrombosis. Other exemplary methods include, but are not limited to, RT-PCR and transcription-mediated amplification (TMA).

The target sequences to be amplified from the subject include AT III, protein C, protein S, fibrinogen, factor V, factor II, ACE, and MTHFR. In particular examples, the VT-associated target sequences to be amplified consist essentially of, or consist only of AT III, protein C, protein S, fibrinogen, factor V, factor II, MTHFR and ACE.

A pair of primers can be utilized in the amplification reaction. One or both of the primers can be labeled, for example with a detectable radiolabel, fluorophore, or biotin molecule. The pair of primers includes an upstream primer (which binds 5′ to the downstream primer) and a downstream primer (which binds 3′ to the upstream primer). The pair of primers used in the amplification reaction are selective primers which permit amplification of a nucleic acid involved in venous thrombosis. Primers can be selected to amplify a nucleic acid molecule listed in Table 1, or represented by those listed in Table 1.

An additional pair of primers can be included in the amplification reaction as an internal control. For example, these primers can be used to amplify a “housekeeping” nucleic acid molecule, and serve to provide confirmation of appropriate amplification. In another example, a target nucleic acid molecule including primer hybridization sites can be constructed and included in the amplification reactor. One of skill in the art will readily be able to identify primer pairs to serve as internal control primers.

Arrays for Detecting Nucleic Acid and Protein Sequences

In particular examples, methods for detecting an abnormality in at least one VT-related gene use the arrays disclosed herein. Such arrays can include nucleic acid molecules. In one example, the array includes nucleic acid oligonucleotide probes that can hybridize to wild-type, mutant, or polymorphic VT gene sequences, such as AT III, protein C, protein S, fibrinogen, factor V, prothrombin (factor II), MTHFR and ACE. In a particular example, an array includes oligonucleotides that can recognize the 143 VT-associated recurrent mutations and polymorphisms listed in Table 1, such as the oligonucleotide probes shown in even numbered SEQ ID NOS: 2-284 and SEQ ID NO: 287. hi other examples, an array includes oligonucleotide probes that can recognize both mutant and wild-type factor V, prothrombin (factor II), AT III, PC, PS, fibrinogen, MTHFR, and ACE sequences, such as SEQ ID NOS: 1-287. Certain of such arrays (as well as the methods described herein) can include VT-related molecules that are not listed in Table 1, as well as other sequences, such as one or more probes that recognize one or more housekeeping genes.

Arrays can be used to detect the presence of amplified sequences involved in venous thrombosis, such as antithrombin III, protein C, protein S, fibrinogen, factor V, prothrombin (factor II), MTHFR and ACE sequences, using specific oligonucleotide probes. The arrays herein termed “VT detection arrays,” are used to determine the genetic susceptibility of a subject to developing venous thrombosis. In one example, a set of oligonucleotide probes is attached to the surface of a solid support for use in detection of the VT-associated sequences, such as those amplified nucleic acid sequences obtained from the subject. Additionally, if an internal control nucleic acid sequence was amplified in the amplification reaction (see above), an oligonucleotide probe can be included to detect the presence of this amplified nucleic acid molecule.

The oligonucleotide probes bound to the array can specifically bind sequences amplified in the amplification reaction (such as under high stringency conditions). Thus, sequences of use with the method are oligonucleotide probes that recognize the VT-related sequences, such as antithrombin III, PC, PS, fibrinogen, factor V, prothrombin (factor II), MTHFR and ACE gene sequences. Such sequences can be determined by examining the sequences of the different species, and choosing primers that specifically anneal to a particular wild-type or mutant sequence (such as those listed in Table 1 or represented by those listed in Table 1), but not others. One of skill in the art will be able to identify other VT-associated oligonucleotide molecules that can be attached to the surface of a solid support for the detection of other amplified VT-associated nucleic acid sequences.

The methods and apparatus in accordance with the present disclosure takes advantage of the fact that under appropriate conditions oligonucleotides form base-paired duplexes with nucleic acid molecules that have a complementary base sequence.

The stability of the duplex is dependent on a number of factors, including the length of the oligonucleotides, the base composition, and the composition of the solution in which hybridization is effected. The effects of base composition on duplex stability may be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mismatched duplexes may be substantially reduced.

The length of each oligonucleotide sequence employed in the array can be selected to optimize binding of target VT-associated nucleic acid sequences. An optimum length for use with a particular VT-associated nucleic acid sequence under specific screening conditions can be determined empirically. Thus, the length for each individual element of the set of oligonucleotide sequences including in the array can be optimized for screening. In one example, oligonucleotide probes are from about 20 to about 35 nucleotides in length or about 25 to about 40 nucleotides in length.

The oligonucleotide probe sequences forming the array can be directly linked to the support, for example via the 5′- or 3′-end of the probe. In one example, the oligonucleotides are bound to the solid support by the 5′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. Alternatively, the oligonucleotide probes can be attached to the support by non-VT-associated sequences such as oligonucleotides or other molecules that serve as spacers or linkers to the solid support.

In another example, an array includes protein sequences, which include at least one VT-related protein such as one encoded by a nucleic acid molecule listed in Table 1 (or genes, cDNAs or other polynucleotide molecules including one of the listed sequences, or a fragment thereof), or a fragment of such protein, or an antibody specific to such a protein or protein fragment. Such arrays can also contain any particular subset of the nucleic acids (or corresponding molecules) listed in Table 1. The proteins or antibodies forming the array can be directly linked to the support. Alternatively, the proteins or antibodies can be attached to the support by spacers or linkers to the solid support.

Abnormalities in VT-related proteins can be detected using, for instance, a VT protein-specific binding agent, which in some instances will be detectably labeled. In certain examples, therefore, detecting an abnormality includes contacting a sample from the subject with a VT protein-specific binding agent; and detecting whether the binding agent is bound by the sample and thereby measuring the levels of the VT-related protein present in the sample, in which a difference in the level of VT-related protein in the sample, relative to the level of VT-related protein found an analogous sample from a subject not predisposed to developing VT, or a standard VT-related protein level in analogous samples from a subject not having a predisposition for developing VT, is an abnormality in that VT-related molecule.

In particular examples, the microarray material is formed from glass (silicon dioxide). Suitable silicon dioxide types for the solid support include, but are not limited to: aluminosilicate, borosilicate, silica, soda lime, zinc titania and fused silica (for example see Schena, Micraoarray Analysis. John Wiley & Sons, Inc, Hoboken, New Jersey, 2003). The attachment of nucleic acids to the surface of the glass can be achieved by methods known in the art, for example by surface treatments that form from an organic polymer. Particular examples include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylefievinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567, herein incorporated by reference), organosilane compounds that provide chemically active amine or aldehyde groups, epoxy or polylysine treatment of the microarray. Another example of a solid support surface is polypropylene.

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides are not amenable to non-specific binding, or when non-specific binding occurs, such materials can be readily removed from the surface without removing the oligonucleotides.

In one example, the surface treatment is amine-containing silane derivatives. Attachment of nucleic acids to an amine surface occurs via interactions between negatively charged phosphate groups on the DNA backbone and positively charged amino groups (Schena, Micraoarray Analysis. John Wiley & Sons, Inc, Hoboken, N.J., 2003, herein incorporated by reference). In another example, reactive aldehyde groups are used as surface treatment. Attachment to the aldehyde surface is achieved by the addition of 5′-amine group or amino linker to the DNA of interest. Binding occurs when the nonbonding electron pair on the amine linker acts as a nucleophile that attacks the electropositive carbon atom of the aldehyde group (Id.).

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185, herein incorporated by reference). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Particularly disclosed for preparation of arrays at this time are biaxially oriented polypropylene (BOPP) films; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789, herein incorporated by reference). In another example, sequences are synthesized directly onto the support to provide the desired array (see U.S. Pat. No. 5,554,501, herein incorporated by reference). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT applications WO 85/01051 and WO 89/10977, or U.S. Pat. No. 5,554,501, herein incorporated by reference).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second (2°) set of rows that are now perpendicular to the first set. This process creates a multiple-channel array whose intersection generates a plurality of discrete cells.

In particular examples, the oligonucleotide probes on the array include one or more labels, that permit detection of oligonucleotide probe:target sequence hybridization complexes.

Detection of Nucleic Acids and Proteins

The nucleic acids and proteins obtained from the subject may contain one or more insertions, deletions, substitutions, or combinations thereof in one or more genes associated with venous thrombosis, such as those listed in Table 1. Such mutations or polymorphisms (or both) can be detected to determine if the subject has a genetic disposition to developing venous thrombosis. Any method of detecting a nucleic acid molecule or protein can be used, such as physical or functional assays.

Methods for labeling nucleic acid molecules and proteins, such that they can be detected, are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I and ³⁵S. For example, radioactive and fluorescent labeling methods, as well as other methods known in the art, are suitable for use with the present disclosure. In one example, the primers used to amplify the subject's nucleic acids are labeled (such as with biotin, a radiolabel, or a fluorophore). In another example, the amplified nucleic acid samples are end-labeled to form labeled amplified material. For example, amplified nucleic acid molecules can be labeled by including labeled nucleotides in the amplification reactions. In a particular example, proteins obtained from a subject are labeled and subsequently analyzed, for example by applying them to an array.

The amplified nucleic acid molecules associated with venous thrombosis are applied to the VT detection array under suitable hybridization conditions to form a hybridization complex. In particular examples, the amplified nucleic acid molecules include a label. In one example, a pre-treatment solution of organic compounds, solutions that include organic compounds, or hot water, can be applied before hybridization (see U.S. Pat. No. 5,985,567, herein incorporated by reference).

Hybridization conditions for a given combination of array and target material can be optimized routinely in an empirical manner close to the Tₘ of the expected duplexes, thereby maximizing the discriminating power of the method. Identification of the location in the array, such as a cell, in which binding occurs, permits a rapid and accurate identification of sequences associated with venous thrombosis present in the amplified material (see below).

The hybridization conditions are selected to permit discrimination between matched and mismatched oligonucleotides. Hybridization conditions can be chosen to correspond to those known to be suitable in standard procedures for hybridization to filters and then optimized for use with the arrays of the disclosure. For example, conditions suitable for hybridization of one type of target would be adjusted for the use of other targets for the array. In particular, temperature is controlled to substantially eliminate formation of duplexes between sequences other than exactly complementary VT-associated wild-type of mutant sequences. A variety of known hybridization solvents can be employed, the choice being dependent on considerations known to one of skill in the art (see U.S. Pat. No. 5,981,185, herein incorporated by reference).

Once the amplified nucleic acid molecules associated with venous thrombosis have been hybridized with the oligonucleotides present in the VT detection array, the presence of the hybridization complex can be analyzed, for example by detecting the complexes.

Detecting a hybridized complex in an array of oligonucleotide probes has been previously described (see U.S. Pat. No. 5,985,567, herein incorporated by reference). In one example, detection includes detecting one or more labels present on the oligonucleotides, the amplified sequences, or both. In particular examples, developing includes applying a buffer. In one embodiment, the buffer is sodium saline citrate, sodium saline phosphate, tetramethylammonium chloride, sodium saline citrate in ethylenediaminetetra-acetic, sodium saline citrate in sodium dodecyl sulfate, sodium saline phosphate in ethylenediaminetetra-acetic, sodium saline phosphate in sodium dodecyl sulfate, tetramethylanmuonium chloride in ethylenediaminetetra-acetic, tetramethylammonium chloride in sodium dodecyl sulfate, or combinations thereof. However, other suitable buffer solutions can also be used.

Detection can further include treating the hybridized complex with a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or colorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when radiolabels are used.

In particular examples, the method further includes quantification, for instance by determining the amount of hybridization.

### Kits

The present disclosure provides for kits that can be used to determine whether a subject, such as an otherwise healthy human subject, is genetically predisposed to venous thrombosis. Such kits allow one to determine if a subject has one or more genetic mutations or polymorphisms in sequences associated with venous thrombosis, including those listed in Table 1.

The disclosed kits include a binding molecule, such as an oligonucleotide probe that selectively hybridizes to a VT-related molecule (such as a mutant or wild-type nucleic acid molecule) that is the target of the kit. In one example, the kit includes the oligonucleotide probes shown in SEQ ID NOS: 1-287, or a subset thereof, such as even-numbered SEQ ID NOS: 2-284 and SEQ ID NO: 287 or odd-numbered SEQ ID NOS: 1-285 and SEQ ID NO: 286. In another example, a kit includes at least 20 of the probes shown in SEQ ID NOS: 1-287, such as at least 50, at least 75, at least 100, at least 125, at least 150, at least 175, at least 200, at least 225, or at least 250 of the probes shown in SEQ ID NOS: 1-287. It is understood that fragments of the full-length probes shown in SEQ ID NOS: 1-287 can also be used, such as fragments that include at least 15 contiguous nucleotides of any of SEQ ID NOS: 1-287, such as at least 16 contiguous nucleotides, such as at least 17 contiguous nucleotides, such as at least 18 contiguous nucleotides, such as at least 19 contiguous nucleotides, such as at least 20 contiguous nucleotides, such as at least 21 contiguous nucleotides, such as at least 22 contiguous nucleotides, such as at least 23 contiguous nucleotides, or such as at least 24 contiguous nucleotides, of any of SEQ ID NOS: 1-287.

In a particular example, kits include antibodies capable of binding to wild-type VT-related proteins or to mutated or polymorphic proteins. Such antibodies have the ability to distinguish between a wild-type and a mutant or polymorphic VT-related protein.

The kit can further include one or more of a buffer solution, a conjugating solution for developing the signal of interest, or a detection reagent for detecting the signal of interest, each in separate packaging, such as a container. In another example, the kit includes a plurality of VT-related target nucleic acid sequences for hybridization with a VT detection array to serve as positive control. The target nucleic acid sequences can include oligonucleotides such as DNA, RNA, and peptide-nucleic acid, or can include PCR fragments.

### Venous Thrombosis Preventative Therapy

The present disclosure also provides methods of avoiding or reducing the incidence of venous thrombosis in a subject determined to be genetically predisposed to developing venous thrombosis. For example, if using the screening methods described above a mutation or polymorphism in at least one VT-related molecule in the subject is detected, a treatment is selected to avoid or reduce the incidence of venous thrombosis or to delay the onset of venous thrombosis. The subject then can be treated in accordance with this selection, for example by administration of one or more anticoagulant agents. In some examples, the treatment selected is specific and tailored for the subject, based on the analysis of that subject's profile for one or more VT-related molecules.

The disclosure is further illustrated by the following non-limiting Examples.

### EXAMPLE 1

### Mutations and Polymorphisms Associated with Venous Thrombosis

Table 1 describes VT-related nucleic acid and protein sequences used to design an array that allows for screening of all currently known 143 venous thrombosis associated recurrent mutations and polymorphisms in eight different genes. However, one skilled in the art will appreciate that additional recurrent VT-associated mutations and polymorphisms not currently identified can also be used. For each potential site of mutation/polymorphism, two oligonucleotide probes were designed (see Example 3).

TABLE 1
Mutations and polymorphisms associated with venous thrombosis.

| Gene | Mutation or polymorphism* |
|---|---|
| AT III | Type I AT III deficiency: 2770insT, 5311-5320del6bp, |
| | 5356-64delCTT, 5381C/T, 5390C/T, 5493A/G, |
| | 6490C/T, 9788G/A, 9819C/T, 13342insA, 13380T/C |
| | Type II AT III deficiency: |
| | RS mutations: 6460A/G, 13262G/A, 13268G/C, |
| | 13268G/T, 13295C/T, 13296G/A and 13299C/T |
| | HBS mutations: 2484T/A, 2586C/T, 2603C/T, |
| | 2604G/A, 2759C/T, 5382G/A |
| | PE mutations: 13324C/A, 13328G/A, 13333C/G, |
| | 13337C/A, 13338C/T, 13392G/C |
| Protein C | Type I PC deficiency: 41G/A; 1357C/T; 1381C/T; |
| | 3103C/T; 3169T/C; 3217G/T; 3222G/A; 3222G/T; |
| | 3359G/A; 3360C/A; 3363/4, insC; 3439C/T; 6128T/C; |
| | 6152C/T; 6182C/T; 6216C/T; 6245C/T; 6246G/A; |
| | 6265G/C; 6274C/T; 7176G/A; 7253C/T; 8403C/T; |
| | 8481A/G; 8485/6 delAC or 8486/7 delCA; 8551C/T; |
| | 8559G/A; 8571C/T; 8572G/A; 8589G/A; 8604G/A; |
| | 8608C/T; 8631C/T; 8678-80 del3nt; 8689T/C; 8695C/T; |
| | 8763G/A; 8857, delG; 8895A/C; 8924C/G |
| | Type II PC deficiency: 1387C/T; 1388G/A; 1432C/T; |
| | 6218C/T; 6219G/A; 7219C/A; 8470G/A; 8744G/A; |
| | 8769C/T; 8790G/A; 8886G/A |
| | PC gene polymorphisms: −1654C/T; −1641A/G, −1476A/T |
| Protein S | Quantitative PS deficiency (type I and type III): −34, TC |
| | (delG); −24, GTG/GAG; 19, GAA/TAA; 26, |
| | GAA/GCA; 44, TA (delCTTA); 46, GTT/CTT; intron d, |
| | G/A, exon 4 +1; 155, AAG/GAG; 217, AAT/AGT; 238, |
| | CAG/TAG; 265, TTT (ins T), 293, TCA/TGA; 295, |
| | GGC/GTC; intron j, G/A, exon 10 +5; 349, GAA/AAA; |
| | 372, delCTTTTT, insAA; intron k, A/G, exon 12 −9; |
| | 405, CTA/CCA; 410, CGA/TGA; 431, AA (insA); 465, |
| | TGG/TGA; 474, CGT/TGT; 522, CAG/TAG; 534, |
| | CTG/CGG; 625, TGT/CGT |
| | Qualitative PS deficiency (type II): −2, CGT/CTT; 9, |
| | AAA/GAA; intron e, G/A, exon 5 +5 |
| | Unknown type of PS deficiency: −25, CT (insT); 467, |
| | GTA/GGA; 633, (delAA); 636, TAA/TAT |
| | PS gene polymorphisms: intron k, C/T, exon 11 +54; |
| | 460, TCC/CCC; 626, CCA/CCG; exon 15, C/A 520 nt |
| | after the stop codon |
| Fibrinogen | α chain: α(16)Arg/Cys; α(16)Arg/His; α(19)Arg/Gly; |
| | α(461)Lys/stop; α(554)Arg/Cys |
| | β chain: β(14)Arg/Cys; β(68)Ala/Thr; β(255)Arg/Cys |
| | γ chain: γ(275)Arg/Cys; γ(275)Arg/His; γ(292)Gly/Val; |
| | γ(308)Asn/Lys; γ(318)Asp/Gly |
| | Fibrinogen gene polymorphism: Thr312Ala |
| Factor V | 1691G/A; 1628G/A; 4070A/G; 1090A/G; 1091G/C |
| Prothrombin | 20210G/A |
| (Factor II) | |
| MTHFR | 677C/T; 1298A/C |
| ACE | Intron 16, 288 bp insertion/deletion |

*Nucleotide or amino acid number refers to the human sequence, although one skilled in the art can determine the corresponding nucleotide or amino acid for other organisms.

### EXAMPLE 2

### Statistical Analysis in the Prediction of Venous Thrombosis

This example demonstrates that MERT offers a high magnitude clinical validity by assessing 143 alleles simultaneously in identifying individuals at very high risk of developing VT, even if the contribution of each allele to the risk is small and not enough to cause VT.

To demonstrate statistically that the disclosed methods can predict a healthy subject's probability of developing venous thrombosis, the following methods were used. The results described below demonstrate that disease prediction for venous thrombosis is greatly improved by considering multiple predisposing genetic factors concurrently. To demonstrate how concurrent screening of multiple venous thrombosis (VT) associated susceptibility gene defects improves the prediction of developing venous thrombosis, likelihood ratios for each VT associated susceptibility gene test were calculated by logistic regression and then the combined likelihood ratio (LR) for the panel of VT associated susceptibility gene tests was calculated simply as the product of the likelihood ratios (LRs) of the individual tests assuming each test is independent.

For the calculations, 10 VT associated susceptibility alleles in eight VT associated genes with an established prevalence both in control subjects and unselected VT patients were selected.

The relevant allele frequencies were derived for AT III, protein C and protein S deficiencies, fibrinogen Thr312Ala, FV Leiden (G1691A), FV G1628A, FV A4070G (R2 allele), prothrombin G20210A, MTHFR C677G, and ACE DD variants using data from previously reported case-control studies conducted in the different ethnic populations regarding VT associated genetic susceptibility (Seligsohn and Lubetsky, N. Engl. J. Med. 344:1222-31, 2001; Heijboer et al. N. Engl. J. Med. 323:1512-6, 1990; Pabinger et al., Blood. Coagul. Fibrinolysis 3:547-53, 1992; Melissari et al., Blood. Coagul. Fibrinolysis 3:749-58, 1992; Bombeli et al. Am. J. Hematol. 70:126-32, 2002; Salomon et al., Arterioscler. Thrombin. Vasc. Biol. 19:511-8, 1999; Harper et al., Br. J. Haematol. 77:360-364, 1991; Tait et al. Br. J. Haematol. 87:106-12, 1994; Arruda et al., Thromb. Haemost. 77:818-21, 1997; Junker et al., Arterioscler. Thromb. Vasc. Biol. 19:2568-72, 1999; Heller et al., Circulation. 108:1362-7, 2003; Jerrard-Dunne et al. Stroke. 34:1821-7, 2003; Patel et al. Thromb. Haemost. 90:835-8, 2003; Shen et al Thromb. Res. 99:447-52, 2000; Sakata et al. J. Thromb. Hemost. 2:528-30, 2004; Lee et al. Ann. Acad. Med. Singapore. 31:761-4, 2002; Liu et al. Thromb. Haemost. 71:416-9, 1994; Suehisa et al. Blood. Coagul. Fibrinolysis. 12:95-9, 2001; Chen et al. Ann. Hematol. 82:114-7, 2003; Ho et al. Am. J. Hematol. 63:74-8, 2000; Miletich et al., N. Engl. J. Med. 317:991-6, 1987; Horellou et al., BMJ289; 1285-1287, 1984; Gladson et al., Thromb. Haemost. 59:18-22, 1988; Tait et al., Thromb. Haemost. 73:87-93, 1995; Dykes et al., Br. J Haematol. 113:636-41; 2001; Carter et al., Blood. 96:1177-9, 2000; Liu Yet al., J. Med. Genet. 38:31-5, 2001; De Stefano et al. Semin. Thromb. Hemost. 24:367-79, 1998; Benson et al. Thromb. Haemost. 86:1188-92, 2001; Ehrenforth et al. Arterioscler. Thromb. Vasc. Biol. 19:276-80, 1999; Leroyer et al. Thromb. Haemost. 80:49-51, 1998; Brown et al. Br. J. Haematol. 98:907-9, 1997; Arruda et al. Thromb. Haemost. 78:1430-3, 1997; de Moerloose et al., Thromb. Haemost. 80:239-41, 1998; Dowling et al. J. Thromb. Hemost. 1:80-7, 2003; Rees et al. Br. J. Haematol. 105:564-6, 1999; Helley et al. Hum. Genet. 100:245-8, 1997; Le et al. Clin. Genet. 57:296-303, 2000; Lu et al., Thromb. Res 107:7-12, 2002; Dilley et al., Am. J. Epidemiol. 147:30-5, 1998; Faisel et al., Eur. J Hum. Genet. 12:187-91, 2004; Dogulu et al., Thromb. Res. 111 :389-95, 2003; Hiyoshi et al. Thromb. Haemost. 80:705-6, 1998; Watanabe et al. Thromb. Haemost. 86:1594-5, 2001; Alhenc-Gelas et al. Thromb. Haemost. 81:193-97, 1999; Poort et al. Blood 88:3698-703, 1996; Hillarp et al. Thromb. Haemost. 78:990-2, 1997; Ferraresi et al. Arterioscler. Thromb. Vasc. Biol. 17:2418-22, 1997; Corral et al. Br. J Haematol. 99:304-307, 1997; Hainaut et al. Acta Clin Belg 53:344-348, 1998; Cumming et al. Br. J Haematol. 98:353-355, 1997; Souto et al. Thromb. Haemost. 80:366-9, 1998; Eichinger et al. Thromb. Haemost. 81:14-7, 1999; Tosetto et al. Thromb. Haemost. 82:1395-98, 1999; Ridker et al. Circulation. 99:999-1004, 1999; Margaglione et al., Thromb. Haemost. 79:907-11, 1998; Dilley et al., J. Lab. Clin. Med. 132:452-5, 1998; Howard et al. Blood 91:1092, 1998; Zheng et al., Br. J. Haematol. 109:870-4, 2000; Lin et al., Thromb. Res. 97:89-94, 2000; Fatini et al., Eur. J. Clin. Invest. 33:642-7, 2003; and Hooper et al., Am. J. Hematol. 70:1-8, 2002) (Table 2).

TABLE 2
Frequency of inherited thrombophilias among control
subjects and unselected patients with VT

| | Control subjects | | | Unselected patients | | |
|---|---|---|---|---|---|---|
| | | Number tested | | | Number tested | |
| | Total Screened | positive | | Total Screened | positive | |
| Antithrombin III | 15,610 | 33 | (0.2%) | 3,509 | 122 | (3.5%) |
| deficiency | | | | | | |
| Protein C deficiency | 21,011 | 45 | (0.2%) | 3,557 | 193 | (5.4%) |
| Protein S deficiency | 5,212 | 28 | (0.5%) | 3,332 | 189 | (5.7%) |
| Fibrinogen gene | 250 ^{I} | 101 | (40.4%) | 218 | 110 | (50.4%) |
| Thr312Ala | 402 ^{II} | 270 | (67.2%) | | | |
| polymorphism | | | | | | |
| FV gene G1691A | 20,313 ^{I} | 1,091 | (5.4%) | 3,651 | 644 | (17.6%) |
| (Leiden) | 8,211 ^{III} | 54 | (0.7%) | | | |
| polymorphism | | | | | | |
| FV gene G1628A | 245 ^{I} | 22 | (9%) | 133 | 26 | (20%) |
| polymorphism | 505 ^{II} | 360 | (72%) | 156 | 145 | (93%) |
| | 245 ^{IV} | 132 | (54%) | | | |
| FV gene A4070G (R2 | 394 ^{I} | 45 | (11.4%) | 205 | 38 | (18.5%) |
| allele) polymorphism | 2,029 ^{V} | 114 | (5.6%) | | | |
| Prothrombin gene | 7,110 ^{I} | 188 | (2.6%) | 4,312 | 222 | (5.1%) |
| G20210A | 2,299 ^{III} | 1 | (0.04%) | | | |
| polymorphism | | | | | | |
| MTHFR gene C677T | 1,222 ^{I} | 146 | (11.9%) | 328 | 62 | (18.9%) |
| polymorphism (TT) | 372 ^{II} | 67 | (18%) | | | |
| ACE gene DD | 378 ^{I} | 101 | (26.7%) | 208 | 99 | (48%) |
| genotype | 370 ^{VI} | 80 | (21.6%) | 184 | 71 | (38.6%) |

^{I} Caucasian subjects
^{II} Asian subjects
^{III} Non-European subjects from Africa, North America, Asia, Australasia, Latin America and Middle East and Inuit subjects.
^{IV} African subjects
^{V} Non-European subjects from North America, Latin America, Asia and Pacific Islands.
^{VI} African-American subjects

The LR calculations were performed by logistic regression. By treating the data retrieved from the previously reported case control studies regarding VT genetic susceptibility in different ethnic populations as a valid estimate of the risk odds ratio, LR for each allele positive test was calculated by exponentiation of the result obtained as previously described (Albert, Clin. Chem. 28:1113-9, 1982; McCullagh and Nelder, Chapman and Hall, London, 1989; Yang et al., Am. J. Hum. Genet., 72:636-49, 2003).

The posterior probability of venous thrombosis (the probability of developing venous thrombosis) was determined for the individuals with allele-positive test results for each genetic test (also known as positive predictive value of each genetic test).

Calculated likelihood ratios and positive predictive values for each venous thrombosis associated susceptibility gene test were demonstrated in Table 3.

TABLE 3
Likelihood ratios and Positive predictive values of single
susceptibility genes and multiple genetic screening with
MERT for developing VT in healthy subjects

| | | Posterior |
|---|---|---|
| | | probability |
| | Likelihood | of developing |
| Single susceptibility test analysis | Ratio | VT |
| Antithrombin III deficiency | 16.4 | 1.6% |
| Protein C deficiency | 25.3 | 2.5% |
| Protein S deficiency | 10.6 | 1.0% |
| Fibrinogen Thr312Ala polymorphism | 1.25 † | 0.12% |
| Factor V gene | | |
| G1691A (Leiden) polymorphism | 3.28 † | 0.33% |
| G1628A polymorphism | 2.18 † | 0.22% |
| | 1.3 ‡ | 0.13% |
| A4070G polymorphism (R2 allele) | 1.62 † | 0.16% |
| Prothrombin gene G20210A | 1.95 † | 0.2% |
| polymorphism | | |
| MTHFR gene C677T polymorphism | 1.58 † | 0.16% |
| (TT) | | |
| ACE DD genotype | 1.78 † | 0.18% |
| | 1.78 * | 0.18% |
| Concurrent screening of 8 genes | 349250.7 † | 99.7% |
| with MERT | 5717.6 ‡ | 85.1% |
| | 7828.7 * | 88.7% |

† Caucasian populations;
‡ Asian populations;
* African populations

Then, assuming that the effect of each of the genetic defects in the eight different genes is independent and that all interactive effects are purely multiplicative, the LR was calculated for the panel of ten VT associated genetic susceptibility tests as the product of the likelihood ratios of the individual test results.

As shown in Table 3, whereas each genetic test provides limited predictive information about the probability of developing venous thrombosis (the posterior probabilities of disease range from 0.12% to 2.5% for each test alone), the posterior probability of venous thrombosis occurring increases to 99.7% when estimated with unselected patients for Caucasians and 85.1% for Asians and 88.7% for African populations by using the disclosed methods, an increase of >30-fold.

### EXAMPLE 3

### Array for Detecting Susceptibility to Venous Thrombosis

For each potential site of mutation/polymorphism (Table 1), two oligonucleotide probes were designed (SEQ ID NOS 1-287). The first is complementary to the wild type sequence (odd numbers of SEQ ID NOS: 1-285 and SEQ ID NO: 286) and the second is complementary to the mutated sequence (even numbers of SEQ ID NOS: 2-284 and SEQ ID NO: 287). For example, SEQ ID NO: 1 is complementary to a wild-type ATIII sequence, and SEQ ID NO: 2 is complementary to a mutant ATIII sequence, which can be used to detect the presence of a “T” insertion at nucleotide 2770. The disclosed oligonucleotide probes can further include one or more detectable labels, to permit detection of hybridization signals between the probe and a target sequence.

Compilation of “loss” and “gain” of hybridization signals will reveal the genetic status of the individual with respect to the 143 known VT-associated recurrent defects.

### EXAMPLE 4

### Nucleic Acid-Based Analysis

The VT-related nucleic acid molecules provided herein can be used in methods of genetic testing for predisposition to venous thrombosis owing to VT-related nucleic acid molecule polymorphism/mutation in comparison to a wild-type nucleic acid molecule. For such procedures, a biological sample of the subject is assayed for a polymorphism or mutation (or both) in a VT-related nucleic acid molecule, such as those listed in Table 1. Suitable biological samples include samples containing genomic DNA or RNA (including mRNA) obtained from cells of a subject, such as those present in peripheral blood, urine, saliva, tissue biopsy, surgical specimen, amniocentesis samples and autopsy material.

The detection in the biological sample of a polymorphism/mutation in one or more VT-related nucleic acid molecules, such as those listed in Table 1, can be achieved by methods such as hybridization using allele specific oligonucleotides (ASOs) (Wallace et al., CSHL Symp. Quant. Biol. 51:257-61, 1986), direct DNA sequencing (Church and Gilbert, Proc. Natl. Acad. Sci. USA 81:1991-1995, 1988), the use of restriction enzymes (Flavell et al., Cell 15:25, 1978; Geever et al., 1981), discrimination on the basis of electrophoretic mobility in gels with denaturing reagent (Myers and Maniatis, Cold Spring Harbor Symp. Quant. Biol. 51:275-84, 1986), RNase protection (Myers et al., Science 230:1242, 1985), chemical cleavage (Cotton et al., Proc. Natl. Acad. Sci. USA 85:4397-401, 1985), and the ligase-mediated detection procedure (Landegren et al., Science 241:1077, 1988).

Oligonucleotides specific to wild-type or mutated VT-related sequences can be chemically synthesized using commercially available machines. These oligonucleotides can then be labeled, for example with radioactive isotopes (such as ³²p) or with non-radioactive labels such as biotin (Ward and Langer et al., Proc. Natl. Acad. Sci. USA 78:6633-6657, 1981) or a fluorophore, and hybridized to individual DNA samples immobilized on membranes or other solid supports by dot-blot or transfer from gels after electrophoresis. These specific sequences are visualized, for example by methods such as autoradiography or fluorometric (Landegren et al., Science 242:229-237, 1989) or colorimetric reactions (Gebeyehu et al., Nucleic Acids Res. 15:4513-4534, 1987). Using an ASO specific for a wild-type allele, the absence of hybridization would indicate a mutation or polymorphism in the particular region of the gene. In contrast, if an ASO specific for a mutant allele hybridizes to a clinical sample then that would indicate the presence of a mutation or polymorphism in the region defined by the ASO.

### EXAMPLE 5

### Protein-Based Analysis

This example describes methods that can be used to detect defects in an amount of a VT-related protein, or to detect changes in the amino acid sequence itself. VT-related protein sequences can be used in methods of genetic testing for predisposition to venous thrombosis owing to VT-related protein polymorphism or mutation (or both) in comparison to a wild-type protein. For such procedures, a biological sample of the subject is assayed for a polymorphism or mutation in a VT-related protein, such as those listed in Table 1. Suitable biological samples include samples containing protein obtained from cells of a subject, such as those present in peripheral blood, urine, saliva, tissue biopsy, surgical specimen, amniocentesis samples and autopsy material.

A decrease in the amount of one or more VT-related proteins in a subject can indicate that the subject has an increased susceptibility to developing VT. Similarly, the presence of one or more mutations or polymorphisms in a VT-related protein in comparison to a wild-type protein can indicate that the subject has an increased susceptibility to developing VT.

The determination of reduced VT-related protein levels, in comparison to such expression in a normal subject (such as a subject not predisposed to developing VT), is an alternative or supplemental approach to the direct determination of the presence of VT-related nucleic acid mutations or polymorphisms by the methods outlined above. The availability of antibodies specific to particular VT-related protein(s) will facilitate the detection and quantitation of cellular VT-related protein(s) by one of a number of immunoassay methods which are well known in the art, such as those presented in Harlow and Lane (Antibodies, A Laboratory Manual, CSHL, New York, 1988). Methods of constructing such antibodies are known in the art.

The determination of the presence of one or more mutations or polymorphisms in a VT-related protein, in comparison to a wild-type VT-related protein, is another alternative or supplemental approach to the direct determination of the presence of VT-related nucleic acid mutations or polymorphisms by the methods outlined above. Antibodies that can distinguish between a mutant or polymorphic protein and a wild-type protein can be prepared using methods known in the art.

Any standard immunoassay format (such as ELISA, Western blot, or RIA assay) can be used to measure VT-related polypeptide or protein levels, and to detect mutations or polymorphisms in VT-related proteins. A comparison to wild-type (normal) VT-related protein levels and a decrease in VT-related polypeptide levels is indicative of predisposition to developing VT. Similarly, the presence of one or more mutant or polymorphic VT-related proteins is indicative of predisposition to developing VT. Immunohistochemical techniques can also be utilized for VT-related polypeptide or protein detection and quantification. For example, a tissue sample can be obtained from a subject, and a section stained for the presence of a wild-type or polymorphic or mutant VT-related protein using the appropriate VT-related protein specific binding agents and any standard detection system (such as one that includes a secondary antibody conjugated to horseradish peroxidase). General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating a VT-related protein, a biological sample of the subject, which sample includes cellular proteins, can be used. Quantitation of a VT-related protein can be achieved by immunoassay and the amount compared to levels of the protein found in cells from a subject not genetically predisposed to developing VT. A significant decrease in the amount of one or more VT-related proteins in the cells of a subject compared to the amount of the same VT-related protein found in normal human cells is usually about a 30% or greater difference. Substantial underexpression of one or more VT-related protein(s) can be indicative of a genetic predisposition to developing VT.

### EXAMPLE 6

### Kits

Kits are provided to determine whether a subject has one or more polymorphisms or mutations in a VT-related nucleic acid sequence (such as kits containing VT detection arrays). Kits are also provided that contain the reagents need to detect hybridization complexes formed between oligonucleotides on an array and VT-related nucleic acids amplified from a subject. These kits can each include instructions, for instance instructions that provide calibration curves or charts to compare with the determined (such as experimentally measured) values.

In one example, the kit includes primers capable of amplifying VT-related nucleic acid molecules, such as those listed in Table 1. In particular examples, the primers are provided suspended in an aqueous solution or as a freeze-dried or lyophilized powder. The container(s) in which the primers are supplied can be any conventional container that is capable of holding the supplied form, for instance, microfuge tubes, ampoules, or bottles. In some applications, pairs of primers are be provided in pre-measured single use amounts in individual, typically disposable, tubes, or equivalent containers.

The amount of each primer supplied in the kit can be any amount, depending for instance on the market to which the product is directed. For instance, if the kit is adapted for research or clinical use, the amount of each oligonucleotide primer provided likely would be an amount sufficient to prime several in vitro amplification reactions. Those of ordinary skill in the art know the amount of oligonucleotide primer that is appropriate for use in a single amplification reaction. General guidelines may for instance be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, N.Y., 1989), and Ausubel et al (In Current Protocols in Molecular Biology, John Wiley & Sons, New York, 1998).

In particular examples, a kit includes an array with oligonucleotides that recognize wild-type, mutant or polymorphic VT-related sequences, such as those listed in Table 1. The array can include other oligonucleotides, for example to serve as negative or positive controls. The oligonucleotides that recognize the wild-type and mutant sequences can be on the same array, or on different arrays. A particular array is disclosed in Example 3. For example, the kit can include the oligonucleotides shown in SEQ ID NOS: 1-287, or subsets thereof, such as at least 10 of the oligonucleotides shown in SEQ ID NOS: 1-287, for example at least 20, at least 50, at least 100, at least 143, or even at least 250 of the oligonucleotides shown in SEQ ID NOS: 1-287. In a particular example, an array includes the odd-numbered SEQ ID NOS: 1-285 (i.e. SEQ ID NOS: 1, 3, 5, 7, etc.) and in some examples also SEQ ID NO: 286, or the even-numbered SEQ ID NOS: 2-284 (i.e. SEQ ID NOS: 2, 4, 6, 8, etc.) and in some examples also SEQ ID NO: 287. However, both such arrays can be included in a single kit.

In some examples, kits further include the reagents necessary to carry out hybridization and detection reactions, including, f6r instance appropriate buffers. Written instructions can also be included.

Kits are also provided for the detection of VT-related protein expression, for instance under expression of a protein encoded for by a nucleic acid molecule listed in Table 1. Such kits include one or more wild-type or mutant AT III, protein C, protein S, fibrinogen, factor V (FV), prothrombin (factor II), MTHFR and ACE proteins (full-length, fragments, or fusions) or specific binding agent (such as a polyclonal or monoclonal antibody or antibody fragment), and can include at least one control. The VT-related protein specific binding agent and control can be contained in separate containers. The kits can also include a means for detecting VT-related protein:agent complexes, for instance the agent may be detectably labeled. If the detectable agent is not labeled, it can be detected by second antibodies or protein A, for example, either of both of which also can be provided in some kits in one or more separate containers. Such techniques are well known.

Additional components in some kits include instructions for carrying out the assay. Instructions permit the tester to determine whether VT-linked expression levels are reduced in comparison to a control sample. Reaction vessels and auxiliary reagents such as chromogens, buffers, enzymes, etc. can also be included in the kits.

In view of the many possible embodiments to which the principles of our invention may be applied, it should be recognized that the illustrated embodiment is only a preferred example of the invention and should not be taken as a limitation on the scope of the invention. Rather, the scope of the invention is defined by the following claims. We therefore claim as our invention all that comes within the scope and spirit of these claims.

# SEQUENCE LIS
#TING
<160> NUMBER OF SEQ ID NOS: 287
<210> SEQ ID NO 1
<211> LENGTH: 25
<212> TYPE: DNA
<213> ORGANISM: Artificial sequence
<220> FEATURE:
<223> OTHER INFORMATION: oligonucleotide probe
<400> SEQUENCE: 1
ccctccagca actgatggag gtacg
#
# 25
<210> SEQ ID NO 2
<211> LENGTH: 25
<212> TYPE: DNA
<213> ORGANISM: Artificial sequence
<220> FEATURE:
<223> OTHER INFORMATION: oligonucleotide probe
<400> SEQUENCE: 2
cctccagcaa cttgatggag gtacg
#
# 25
<210> SEQ ID NO 3
<211> LENGTH: 25
<212> TYPE: DNA
<213> ORGANISM: Artificial sequence
<220> FEATURE:
<223> OTHER INFORMATION: oligonucleotide probe
<400> SEQUENCE: 3
tataggtatt taagtttgac accat
#
# 25
<210> SEQ ID NO 4
<211> LENGTH: 25
<212> TYPE: DNA
<213> ORGANISM: Artificial sequence
<220> FEATURE:
<223> OTHER INFORMATION: oligonucleotide probe
<400> SEQUENCE: 4
ttctataggt atttgacacc atatc
#
# 25
<210> SEQ ID NO 5
<211> LENGTH: 25
<212> TYPE: DNA
<213> ORGANISM: Artificial sequence
<220> FEATURE:
<223> OTHER INFORMATION: oligonucleotide probe
<400> SEQUENCE: 5
cagatccact tcttctttgc caaac
#
# 25
<210> SEQ ID NO 6
<211> LENGTH: 25
<212> TYPE: DNA
<213> ORGANISM: Artificial sequence
<220> FEATURE:
<223> OTHER INFORMATION: oligonucleotide probe
<400> SEQUENCE: 6
atcagatcca cttctttgcc aaact
#
# 25
<210> SEQ ID NO 7
<211> LENGTH: 25
<212> TYPE: DNA
<213> ORGANISM: Artificial sequence
<220> FEATURE:
<223> OTHER INFORMATION: oligonucleotide probe
<400> SEQUENCE: 7
aaactgaact gccgactcta tcgaa
#
# 25
<210> SEQ ID NO 8
<211> LENGTH: 25
<212> TYPE: DNA
<213> ORGANISM: Artificial sequence
<220> FEATURE:
<223> OTHER INFORMATION: oligonucleotide probe
<400> SEQUENCE: 8
aaactgaact gctgactcta tcgaa
#
# 25
<210> SEQ ID NO 9
<211> LENGTH: 25
<212> TYPE: DNA
<213> ORGANISM: Artificial sequence
<220> FEATURE:
<223> OTHER INFORMATION: oligonucleotide probe
<400> SEQUENCE: 9
tgccgactct atcgaaaagc caaca
#
# 25
<210> SEQ ID NO 10
<211> LENGTH: 25
<212> TYPE: DNA
<213> ORGANISM: Artificial sequence
<220> FEATURE:
<223> OTHER INFORMATION: oligonucleotide probe
<400> SEQUENCE: 10
tgccgactct attgaaaagc caaca
#
# 25
<210> SEQ ID NO 11
<211> LENGTH: 25
<212> TYPE: DNA
<213> ORGANISM: Artificial sequence
<220> FEATURE:
<223> OTHER INFORMATION: oligonucleotide probe
<400> SEQUENCE: 11
gtgagttggt atatggagcc aagct
#
# 25
<210> SEQ ID NO 12
<211> LENGTH: 25
<212> TYPE: DNA
<213> ORGANISM: Artificial sequence
<220> FEATURE:
<223> OTHER INFORMATION: oligonucleotide probe
<400> SEQUENCE: 12
gtgagttggt atgtggagcc aagct
#
# 25
<210> SEQ ID NO 13
<211> LENGTH: 25
<212> TYPE: DNA
<213> ORGANISM: Artificial sequence
<220> FEATURE:
<223> OTHER INFORMATION: oligonucleotide probe
<400> SEQUENCE: 13
aagaccgaag gccgaatcac cgatg
#
# 25
<210> SEQ ID NO 14
<211> LENGTH: 25
<212> TYPE: DNA
<213> ORGANISM: Artificial sequence
<220> FEATURE:
<223> OTHER INFORMATION: oligonucleotide probe
<400> SEQUENCE: 14
aagaccgaag gctgaatcac cgatg
#
# 25
<210> SEQ ID NO 15
<211> LENGTH: 25
<212> TYPE: DNA
<213> ORGANISM: Artificial sequence
<220> FEATURE:
<223> OTHER INFORMATION: oligonucleotide probe
<400> SEQUENCE: 15
aaagacttct ccggtcttcc ttcca
#
# 25
<210> SEQ ID NO 16
<211> LENGTH: 25
<212> TYPE: DNA
<213> ORGANISM: Artificial sequence
<220> FEATURE:
<223> OTHER INFORMATION: oligonucleotide probe
<400> SEQUENCE: 16
aaagacttct ccagtcttcc ttcca
#
# 25
<210> SEQ ID NO 17
<211> LENGTH: 25
<212> TYPE: DNA
<213> ORGANISM: Artificial sequence
<220> FEATURE:
<223> OTHER INFORMATION: oligonucleotide probe
<400> SEQUENCE: 17
gttgcagaag gccgagatga cctct
#
# 25
<210> SEQ ID NO 18
<211> LENGTH: 25
<212> TYPE: DNA
<213> ORGANISM: Artificial sequence
<220> FEATURE:
<223> OTHER INFORMATION: oligonucleotide probe
<400> SEQUENCE: 18
gttgcagaag gctgagatga cctct
#
# 25
<210> SEQ ID NO 19
<211> LENGTH: 25
<212> TYPE: DNA
<213> ORGANISM: Artificial sequence
<220> FEATURE:
<223> OTHER INFORMATION: oligonucleotide probe
<400> SEQUENCE: 19
caacaggcct ttcctggttt ttata
#
# 25
<210> SEQ ID NO 20
<211> LENGTH: 25
<212> TYPE: DNA
<213> ORGANISM: Artificial sequence
<220> FEATURE:
<223> OTHER INFORMATION: oligonucleotide probe
<400> SEQUENCE: 20
caacaggcct ttacctggtt tttat
#
# 25
<210> SEQ ID NO 21
<211> LENGTH: 25
<212> TYPE: DNA
<213> ORGANISM: Artificial sequence
<220> FEATURE:
<223> OTHER INFORMATION: oligonucleotide probe
<400> SEQUENCE: 21
tgaacactat tatcttcatg ggcag
#
# 25
<210> SEQ ID NO 22
<211> LENGTH: 25
<212> TYPE: DNA
<213> ORGANISM: Artificial sequence
<220> FEATURE:
<223> OTHER INFORMATION: oligonucleotide probe
<400> SEQUENCE: 22
tgaacactat taccttcatg ggcag
#
# 25
<210> SEQ ID NO 23
<211> LENGTH: 25
<212> TYPE: DNA
<213> ORGANISM: Artificial sequence
<220> FEATURE:
<223> OTHER INFORMATION: oligonucleotide probe
<400> SEQUENCE: 23
agagcggcca tcaacaaatg ggtgt
#
# 25
<210> SEQ ID NO 24
<211> LENGTH: 25
<212> TYPE: DNA
<213> ORGANISM: Artificial sequence
<220> FEATURE:
<223> OTHER INFORMATION: oligonucleotide probe
<400> SEQUENCE: 24
agagcggcca tcgacaaatg ggtgt
#
# 25
<210> SEQ ID NO 25
<211> LENGTH: 25
<212> TYPE: DNA
<213> ORGANISM: Artificial sequence
<220> FEATURE:
<223> OTHER INFORMATION: oligonucleotide probe
<400> SEQUENCE: 25
gaaggcagtg aagcagctgc aagta
#
# 25
<210> SEQ ID NO 26
<211> LENGTH: 25
<212> TYPE: DNA
<213> ORGANISM: Artificial sequence
<220> FEATURE:
<223> OTHER INFORMATION: oligonucleotide probe
<400> SEQUENCE: 26
gaaggcagtg aaacagctgc aagta
#
# 25
<210> SEQ ID NO 27
<211> LENGTH: 25
<212> TYPE: DNA
<213> ORGANISM: Artificial sequence
<220> FEATURE:
<223> OTHER INFORMATION: oligonucleotide probe
<400> SEQUENCE: 27
agtgaagcag ctgcaagtac cgctg
#
# 25
<210> SEQ ID NO 28
<211> LENGTH: 25
<212> TYPE: DNA
<213> ORGANISM: Artificial sequence
<220> FEATURE:
<223> OTHER INFORMATION: oligonucleotide probe
<400> SEQUENCE: 28
agtgaagcag ctccaagtac cgctg
#
# 25
<210> SEQ ID NO 29
<211> LENGTH: 25
<212> TYPE: DNA
<213> ORGANISM: Artificial sequence
<220> FEATURE:
<223> OTHER INFORMATION: oligonucleotide probe
<400> SEQUENCE: 29
agtgaagcag ctgcaagtac cgctg
#
# 25
<210> SEQ ID NO 30
<211> LENGTH: 25
<212> TYPE: DNA
<213> ORGANISM: Artificial sequence
<220> FEATURE:
<223> OTHER INFORMATION: oligonucleotide probe
<400> SEQUENCE: 30
agtgaagcag cttcaagtac cgctg
#
# 25
<210> SEQ ID NO 31
<211> LENGTH: 25
<212> TYPE: DNA
<213> ORGANISM: Artificial sequence
<220> FEATURE:
<223> OTHER INFORMATION: oligonucleotide probe
<400> SEQUENCE: 31
gtgattgctg gccgttcgct aaacc
#
# 25
<210> SEQ ID NO 32
<211> LENGTH: 25
<212> TYPE: DNA
<213> ORGANISM: Artificial sequence
<220> FEATURE:
<223> OTHER INFORMATION: oligonucleotide probe
<400> SEQUENCE: 32
gtgattgctg gctgttcgct aaacc
#
# 25
<210> SEQ ID NO 33
<211> LENGTH: 25
<212> TYPE: DNA
<213> ORGANISM: Artificial sequence
<220> FEATURE:
<223> OTHER INFORMATION: oligonucleotide probe
<400> SEQUENCE: 33
tgattgctgg ccgttcgcta aaccc
#
# 25
<210> SEQ ID NO 34
<211> LENGTH: 25
<212> TYPE: DNA
<213> ORGANISM: Artificial sequence
<220> FEATURE:
<223> OTHER INFORMATION: oligonucleotide probe
<400> SEQUENCE: 34
tgattgctgg ccattcgcta aaccc
#
# 25
<210> SEQ ID NO 35
<211> LENGTH: 25
<212> TYPE: DNA
<213> ORGANISM: Artificial sequence
<220> FEATURE:
<223> OTHER INFORMATION: oligonucleotide probe
<400> SEQUENCE: 35
ttgctggccg ttcgctaaac cccaa
#
# 25
<210> SEQ ID NO 36
<211> LENGTH: 25
<212> TYPE: DNA
<213> ORGANISM: Artificial sequence
<220> FEATURE:
<223> OTHER INFORMATION: oligonucleotide probe
<400> SEQUENCE: 36
ttgctggccg tttgctaaac cccaa
#
# 25
<210> SEQ ID NO 37
<211> LENGTH: 25
<212> TYPE: DNA
<213> ORGANISM: Artificial sequence
<220> FEATURE:
<223> OTHER INFORMATION: oligonucleotide probe
<400> SEQUENCE: 37
gccctgtgga catctgcaca gccaa
#
# 25
<210> SEQ ID NO 38
<211> LENGTH: 25
<212> TYPE: DNA
<213> ORGANISM: Artificial sequence
<220> FEATURE:
<223> OTHER INFORMATION: oligonucleotide probe
<400> SEQUENCE: 38
gccctgtgga caactgcaca gccaa
#
# 25
<210> SEQ ID NO 39
<211> LENGTH: 25
<212> TYPE: DNA
<213> ORGANISM: Artificial sequence
<220> FEATURE:
<223> OTHER INFORMATION: oligonucleotide probe
<400> SEQUENCE: 39
aacagaagat cccggaggcc accaa
#
# 25
<210> SEQ ID NO 40
<211> LENGTH: 25
<212> TYPE: DNA
<213> ORGANISM: Artificial sequence
<220> FEATURE:
<223> OTHER INFORMATION: oligonucleotide probe
<400> SEQUENCE: 40
aacagaagat cctggaggcc accaa
#
# 25
<210> SEQ ID NO 41
<211> LENGTH: 25
<212> TYPE: DNA
<213> ORGANISM: Artificial sequence
<220> FEATURE:
<223> OTHER INFORMATION: oligonucleotide probe
<400> SEQUENCE: 41
gccaccaacc ggcgtgtctg ggaac
#
# 25
<210> SEQ ID NO 42
<211> LENGTH: 25
<212> TYPE: DNA
<213> ORGANISM: Artificial sequence
<220> FEATURE:
<223> OTHER INFORMATION: oligonucleotide probe
<400> SEQUENCE: 42
gccaccaacc ggtgtgtctg ggaac
#
# 25
<210> SEQ ID NO 43
<211> LENGTH: 25
<212> TYPE: DNA
<213> ORGANISM: Artificial sequence
<220> FEATURE:
<223> OTHER INFORMATION: oligonucleotide probe
<400> SEQUENCE: 43
ccaccaaccg gcgtgtctgg gaact
#
# 25
<210> SEQ ID NO 44
<211> LENGTH: 25
<212> TYPE: DNA
<213> ORGANISM: Artificial sequence
<220> FEATURE:
<223> OTHER INFORMATION: oligonucleotide probe
<400> SEQUENCE: 44
ccaccaaccg gcatgtctgg gaact
#
# 25
<210> SEQ ID NO 45
<211> LENGTH: 25
<212> TYPE: DNA
<213> ORGANISM: Artificial sequence
<220> FEATURE:
<223> OTHER INFORMATION: oligonucleotide probe
<400> SEQUENCE: 45
tgtaatgaca ccctccagca actga
#
# 25
<210> SEQ ID NO 46
<211> LENGTH: 25
<212> TYPE: DNA
<213> ORGANISM: Artificial sequence
<220> FEATURE:
<223> OTHER INFORMATION: oligonucleotide probe
<400> SEQUENCE: 46
tgtaatgaca ccttccagca actga
#
# 25
<210> SEQ ID NO 47
<211> LENGTH: 25
<212> TYPE: DNA
<213> ORGANISM: Artificial sequence
<220> FEATURE:
<223> OTHER INFORMATION: oligonucleotide probe
<400> SEQUENCE: 47
aactgaactg ccgactctat cgaaa
#
# 25
<210> SEQ ID NO 48
<211> LENGTH: 25
<212> TYPE: DNA
<213> ORGANISM: Artificial sequence
<220> FEATURE:
<223> OTHER INFORMATION: oligonucleotide probe
<400> SEQUENCE: 48
aactgaactg ccaactctat cgaaa
#
# 25
<210> SEQ ID NO 49
<211> LENGTH: 25
<212> TYPE: DNA
<213> ORGANISM: Artificial sequence
<220> FEATURE:
<223> OTHER INFORMATION: oligonucleotide probe
<400> SEQUENCE: 49
cagggtgact ttcaaggcca acagg
#
# 25
<210> SEQ ID NO 50
<211> LENGTH: 25
<212> TYPE: DNA
<213> ORGANISM: Artificial sequence
<220> FEATURE:
<223> OTHER INFORMATION: oligonucleotide probe
<400> SEQUENCE: 50
cagggtgact ttaaaggcca acagg
#
# 25
<210> SEQ ID NO 51
<211> LENGTH: 25
<212> TYPE: DNA
<213> ORGANISM: Artificial sequence
<220> FEATURE:
<223> OTHER INFORMATION: oligonucleotide probe
<400> SEQUENCE: 51
gtgactttca aggccaacag gcctt
#
# 25
<210> SEQ ID NO 52
<211> LENGTH: 25
<212> TYPE: DNA
<213> ORGANISM: Artificial sequence
<220> FEATURE:
<223> OTHER INFORMATION: oligonucleotide probe
<400> SEQUENCE: 52
gtgactttca agaccaacag gcctt
#
# 25
<210> SEQ ID NO 53
<211> LENGTH: 25
<212> TYPE: DNA
<213> ORGANISM: Artificial sequence
<220> FEATURE:
<223> OTHER INFORMATION: oligonucleotide probe
<400> SEQUENCE: 53
tttcaaggcc aacaggcctt tcctg
#
# 25
<210> SEQ ID NO 54
<211> LENGTH: 25
<212> TYPE: DNA
<213> ORGANISM: Artificial sequence
<220> FEATURE:
<223> OTHER INFORMATION: oligonucleotide probe
<400> SEQUENCE: 54
tttcaaggcc aagaggcctt tcctg
#
# 25
<210> SEQ ID NO 55
<211> LENGTH: 25
<212> TYPE: DNA
<213> ORGANISM: Artificial sequence
<220> FEATURE:
<223> OTHER INFORMATION: oligonucleotide probe
<400> SEQUENCE: 55
aaggccaaca ggcctttcct ggttt
#
# 25
<210> SEQ ID NO 56
<211> LENGTH: 25
<212> TYPE: DNA
<213> ORGANISM: Artificial sequence
<220> FEATURE:
<223> OTHER INFORMATION: oligonucleotide probe
<400> SEQUENCE: 56
aaggccaaca ggactttcct ggttt
#
# 25
<210> SEQ ID NO 57
<211> LENGTH: 25
<212> TYPE: DNA
<213> ORGANISM: Artificial sequence
<220> FEATURE:
<223> OTHER INFORMATION: oligonucleotide probe
<400> SEQUENCE: 57
aggccaacag gcctttcctg gtttt
#
# 25
<210> SEQ ID NO 58
<211> LENGTH: 25
<212> TYPE: DNA
<213> ORGANISM: Artificial sequence
<220> FEATURE:
<223> OTHER INFORMATION: oligonucleotide probe
<400> SEQUENCE: 58
aggccaacag gcttttcctg gtttt
#
# 25
<210> SEQ ID NO 59
<211> LENGTH: 25
<212> TYPE: DNA
<213> ORGANISM: Artificial sequence
<220> FEATURE:
<223> OTHER INFORMATION: oligonucleotide probe
<400> SEQUENCE: 59
tcttcatggg cagagtagcc aaccc
#
# 25
<210> SEQ ID NO 60
<211> LENGTH: 25
<212> TYPE: DNA
<213> ORGANISM: Artificial sequence
<220> FEATURE:
<223> OTHER INFORMATION: oligonucleotide probe
<400> SEQUENCE: 60
tcttcatggg cacagtagcc aaccc
#
# 25
<210> SEQ ID NO 61
<211> LENGTH: 25
<212> TYPE: DNA
<213> ORGANISM: Artificial sequence
<220> FEATURE:
<223> OTHER INFORMATION: oligonucleotide probe
<400> SEQUENCE: 61
tcgtggccac ctggggaatt tccgg
#
# 25
<210> SEQ ID NO 62
<211> LENGTH: 25
<212> TYPE: DNA
<213> ORGANISM: Artificial sequence
<220> FEATURE:
<223> OTHER INFORMATION: oligonucleotide probe
<400> SEQUENCE: 62
tcgtggccac ctagggaatt tccgg
#
# 25
<210> SEQ ID NO 63
<211> LENGTH: 25
<212> TYPE: DNA
<213> ORGANISM: Artificial sequence
<220> FEATURE:
<223> OTHER INFORMATION: oligonucleotide probe
<400> SEQUENCE: 63
tccagcagcg agcgtgccca ccagg
#
# 25
<210> SEQ ID NO 64
<211> LENGTH: 25
<212> TYPE: DNA
<213> ORGANISM: Artificial sequence
<220> FEATURE:
<223> OTHER INFORMATION: oligonucleotide probe
<400> SEQUENCE: 64
tccagcagcg agtgtgccca ccagg
#
# 25
<210> SEQ ID NO 65
<211> LENGTH: 25
<212> TYPE: DNA
<213> ORGANISM: Artificial sequence
<220> FEATURE:
<223> OTHER INFORMATION: oligonucleotide probe
<400> SEQUENCE: 65
gtgctgcgga tccgcaaacg tgcca
#
# 25
<210> SEQ ID NO 66
<211> LENGTH: 25
<212> TYPE: DNA
<213> ORGANISM: Artificial sequence
<220> FEATURE:
<223> OTHER INFORMATION: oligonucleotide probe
<400> SEQUENCE: 66
gtgctgcgga tctgcaaacg tgcca
#
# 25
<210> SEQ ID NO 67
<211> LENGTH: 25
<212> TYPE: DNA
<213> ORGANISM: Artificial sequence
<220> FEATURE:
<223> OTHER INFORMATION: oligonucleotide probe
<400> SEQUENCE: 67
tgcttggtct tgcccttgga gcacc
#
# 25
<210> SEQ ID NO 68
<211> LENGTH: 25
<212> TYPE: DNA
<213> ORGANISM: Artificial sequence
<220> FEATURE:
<223> OTHER INFORMATION: oligonucleotide probe
<400> SEQUENCE: 68
tgcttggtct tgtccttgga gcacc
#
# 25
<210> SEQ ID NO 69
<211> LENGTH: 25
<212> TYPE: DNA
<213> ORGANISM: Artificial sequence
<220> FEATURE:
<223> OTHER INFORMATION: oligonucleotide probe
<400> SEQUENCE: 69
ggcatcggca gcttcagctg cgact
#
# 25
<210> SEQ ID NO 70
<211> LENGTH: 25
<212> TYPE: DNA
<213> ORGANISM: Artificial sequence
<220> FEATURE:
<223> OTHER INFORMATION: oligonucleotide probe
<400> SEQUENCE: 70
ggcatcggca gcctcagctg cgact
#
# 25
<210> SEQ ID NO 71
<211> LENGTH: 25
<212> TYPE: DNA
<213> ORGANISM: Artificial sequence
<220> FEATURE:
<223> OTHER INFORMATION: oligonucleotide probe
<400> SEQUENCE: 71
ttctgccagc gcggtgaggg ggaga
#
# 25
<210> SEQ ID NO 72
<211> LENGTH: 25
<212> TYPE: DNA
<213> ORGANISM: Artificial sequence
<220> FEATURE:
<223> OTHER INFORMATION: oligonucleotide probe
<400> SEQUENCE: 72
ttctgccagc gctgtgaggg ggaga
#
# 25
<210> SEQ ID NO 73
<211> LENGTH: 25
<212> TYPE: DNA
<213> ORGANISM: Artificial sequence
<220> FEATURE:
<223> OTHER INFORMATION: oligonucleotide probe
<400> SEQUENCE: 73
ccagcgcggt gagggggaga ggtgg
#
# 25
<210> SEQ ID NO 74
<211> LENGTH: 25
<212> TYPE: DNA
<213> ORGANISM: Artificial sequence
<220> FEATURE:
<223> OTHER INFORMATION: oligonucleotide probe
<400> SEQUENCE: 74
ccagcgcggt gaaggggaga ggtgg
#
# 25
<210> SEQ ID NO 75
<211> LENGTH: 25
<212> TYPE: DNA
<213> ORGANISM: Artificial sequence
<220> FEATURE:
<223> OTHER INFORMATION: oligonucleotide probe
<400> SEQUENCE: 75
ccagcgcggt gagggggaga ggtgg
#
# 25
<210> SEQ ID NO 76
<211> LENGTH: 25
<212> TYPE: DNA
<213> ORGANISM: Artificial sequence
<220> FEATURE:
<223> OTHER INFORMATION: oligonucleotide probe
<400> SEQUENCE: 76
ccagcgcggt gatggggaga ggtgg
#
# 25
<210> SEQ ID NO 77
<211> LENGTH: 25
<212> TYPE: DNA
<213> ORGANISM: Artificial sequence
<220> FEATURE:
<223> OTHER INFORMATION: oligonucleotide probe
<400> SEQUENCE: 77
acaacggcgg ctgcacgcat tactg
#
# 25
<210> SEQ ID NO 78
<211> LENGTH: 25
<212> TYPE: DNA
<213> ORGANISM: Artificial sequence
<220> FEATURE:
<223> OTHER INFORMATION: oligonucleotide probe
<400> SEQUENCE: 78
acaacggcgg ctacacgcat tactg
#
# 25
<210> SEQ ID NO 79
<211> LENGTH: 25
<212> TYPE: DNA
<213> ORGANISM: Artificial sequence
<220> FEATURE:
<223> OTHER INFORMATION: oligonucleotide probe
<400> SEQUENCE: 79
caacggcggc tgcacgcatt actgc
#
# 25
<210> SEQ ID NO 80
<211> LENGTH: 25
<212> TYPE: DNA
<213> ORGANISM: Artificial sequence
<220> FEATURE:
<223> OTHER INFORMATION: oligonucleotide probe
<400> SEQUENCE: 80
caacggcggc tgaacgcatt actgc
#
# 25
<210> SEQ ID NO 81
<211> LENGTH: 25
<212> TYPE: DNA
<213> ORGANISM: Artificial sequence
<220> FEATURE:
<223> OTHER INFORMATION: oligonucleotide probe
<400> SEQUENCE: 81
ggcggctgca cgcattactg cctag
#
# 25
<210> SEQ ID NO 82
<211> LENGTH: 25
<212> TYPE: DNA
<213> ORGANISM: Artificial sequence
<220> FEATURE:
<223> OTHER INFORMATION: oligonucleotide probe
<400> SEQUENCE: 82
ggcggctgca cgccattact gccta
#
# 25
<210> SEQ ID NO 83
<211> LENGTH: 25
<212> TYPE: DNA
<213> ORGANISM: Artificial sequence
<220> FEATURE:
<223> OTHER INFORMATION: oligonucleotide probe
<400> SEQUENCE: 83
gacgacctcc tgcagtgtca ccccg
#
# 25
<210> SEQ ID NO 84
<211> LENGTH: 25
<212> TYPE: DNA
<213> ORGANISM: Artificial sequence
<220> FEATURE:
<223> OTHER INFORMATION: oligonucleotide probe
<400> SEQUENCE: 84
gacgacctcc tgtagtgtca ccccg
#
# 25
<210> SEQ ID NO 85
<211> LENGTH: 25
<212> TYPE: DNA
<213> ORGANISM: Artificial sequence
<220> FEATURE:
<223> OTHER INFORMATION: oligonucleotide probe
<400> SEQUENCE: 85
acctcagtga agttcccttg tggga
#
# 25
<210> SEQ ID NO 86
<211> LENGTH: 25
<212> TYPE: DNA
<213> ORGANISM: Artificial sequence
<220> FEATURE:
<223> OTHER INFORMATION: oligonucleotide probe
<400> SEQUENCE: 86
acctcagtga agctcccttg tggga
#
# 25
<210> SEQ ID NO 87
<211> LENGTH: 25
<212> TYPE: DNA
<213> ORGANISM: Artificial sequence
<220> FEATURE:
<223> OTHER INFORMATION: oligonucleotide probe
<400> SEQUENCE: 87
aggccctgga agcggatgga gaaga
#
# 25
<210> SEQ ID NO 88
<211> LENGTH: 25
<212> TYPE: DNA
<213> ORGANISM: Artificial sequence
<220> FEATURE:
<223> OTHER INFORMATION: oligonucleotide probe
<400> SEQUENCE: 88
aggccctgga agtggatgga gaaga
#
# 25
<210> SEQ ID NO 89
<211> LENGTH: 25
<212> TYPE: DNA
<213> ORGANISM: Artificial sequence
<220> FEATURE:
<223> OTHER INFORMATION: oligonucleotide probe
<400> SEQUENCE: 89
agtcacctga aacgagacac agaag
#
# 25
<210> SEQ ID NO 90
<211> LENGTH: 25
<212> TYPE: DNA
<213> ORGANISM: Artificial sequence
<220> FEATURE:
<223> OTHER INFORMATION: oligonucleotide probe
<400> SEQUENCE: 90
agtcacctga aatgagacac agaag
#
# 25
<210> SEQ ID NO 91
<211> LENGTH: 25
<212> TYPE: DNA
<213> ORGANISM: Artificial sequence
<220> FEATURE:
<223> OTHER INFORMATION: oligonucleotide probe
<400> SEQUENCE: 91
accaagtaga tccgcggctc attga
#
# 25
<210> SEQ ID NO 92
<211> LENGTH: 25
<212> TYPE: DNA
<213> ORGANISM: Artificial sequence
<220> FEATURE:
<223> OTHER INFORMATION: oligonucleotide probe
<400> SEQUENCE: 92
accaagtaga tctgcggctc attga
#
# 25
<210> SEQ ID NO 93
<211> LENGTH: 25
<212> TYPE: DNA
<213> ORGANISM: Artificial sequence
<220> FEATURE:
<223> OTHER INFORMATION: oligonucleotide probe
<400> SEQUENCE: 93
aagatgacca ggcggggaga cagcc
#
# 25
<210> SEQ ID NO 94
<211> LENGTH: 25
<212> TYPE: DNA
<213> ORGANISM: Artificial sequence
<220> FEATURE:
<223> OTHER INFORMATION: oligonucleotide probe
<400> SEQUENCE: 94
aagatgacca ggtggggaga cagcc
#
# 25
<210> SEQ ID NO 95
<211> LENGTH: 25
<212> TYPE: DNA
<213> ORGANISM: Artificial sequence
<220> FEATURE:
<223> OTHER INFORMATION: oligonucleotide probe
<400> SEQUENCE: 95
agatgaccag gcggggagac agccc
#
# 25
<210> SEQ ID NO 96
<211> LENGTH: 25
<212> TYPE: DNA
<213> ORGANISM: Artificial sequence
<220> FEATURE:
<223> OTHER INFORMATION: oligonucleotide probe
<400> SEQUENCE: 96
agatgaccag gcagggagac agccc
#
# 25
<210> SEQ ID NO 97
<211> LENGTH: 25
<212> TYPE: DNA
<213> ORGANISM: Artificial sequence
<220> FEATURE:
<223> OTHER INFORMATION: oligonucleotide probe
<400> SEQUENCE: 97
cagcccctgg caggtgggag gcgag
#
# 25
<210> SEQ ID NO 98
<211> LENGTH: 25
<212> TYPE: DNA
<213> ORGANISM: Artificial sequence
<220> FEATURE:
<223> OTHER INFORMATION: oligonucleotide probe
<400> SEQUENCE: 98
cagcccctgg cacgtgggag gcgag
#
# 25
<210> SEQ ID NO 99
<211> LENGTH: 25
<212> TYPE: DNA
<213> ORGANISM: Artificial sequence
<220> FEATURE:
<223> OTHER INFORMATION: oligonucleotide probe
<400> SEQUENCE: 99
gcaggtggga ggcgaggcag caccg
#
# 25
<210> SEQ ID NO 100
<211> LENGTH: 25
<212> TYPE: DNA
<213> ORGANISM: Artificial sequence
<220> FEATURE:
<223> OTHER INFORMATION: oligonucleotide probe
<400> SEQUENCE: 100
gcaggtggga ggtgaggcag caccg
#
# 25
<210> SEQ ID NO 101
<211> LENGTH: 25
<212> TYPE: DNA
<213> ORGANISM: Artificial sequence
<220> FEATURE:
<223> OTHER INFORMATION: oligonucleotide probe
<400> SEQUENCE: 101
agctggcctg cggggcagtg ctcat
#
# 25
<210> SEQ ID NO 102
<211> LENGTH: 25
<212> TYPE: DNA
<213> ORGANISM: Artificial sequence
<220> FEATURE:
<223> OTHER INFORMATION: oligonucleotide probe
<400> SEQUENCE: 102
agctggcctg cgaggcagtg ctcat
#
# 25
<210> SEQ ID NO 103
<211> LENGTH: 25
<212> TYPE: DNA
<213> ORGANISM: Artificial sequence
<220> FEATURE:
<223> OTHER INFORMATION: oligonucleotide probe
<400> SEQUENCE: 103
ctccttgtca ggcttggtat gggct
#
# 25
<210> SEQ ID NO 104
<211> LENGTH: 25
<212> TYPE: DNA
<213> ORGANISM: Artificial sequence
<220> FEATURE:
<223> OTHER INFORMATION: oligonucleotide probe
<400> SEQUENCE: 104
ctccttgtca ggtttggtat gggct
#
# 25
<210> SEQ ID NO 105
<211> LENGTH: 25
<212> TYPE: DNA
<213> ORGANISM: Artificial sequence
<220> FEATURE:
<223> OTHER INFORMATION: oligonucleotide probe
<400> SEQUENCE: 105
tatgacctgc ggcgctggga gaagt
#
# 25
<210> SEQ ID NO 106
<211> LENGTH: 25
<212> TYPE: DNA
<213> ORGANISM: Artificial sequence
<220> FEATURE:
<223> OTHER INFORMATION: oligonucleotide probe
<400> SEQUENCE: 106
tatgacctgc ggtgctggga gaagt
#
# 25
<210> SEQ ID NO 107
<211> LENGTH: 25
<212> TYPE: DNA
<213> ORGANISM: Artificial sequence
<220> FEATURE:
<223> OTHER INFORMATION: oligonucleotide probe
<400> SEQUENCE: 107
agcaccaccg acaatgacat cgcac
#
# 25
<210> SEQ ID NO 108
<211> LENGTH: 25
<212> TYPE: DNA
<213> ORGANISM: Artificial sequence
<220> FEATURE:
<223> OTHER INFORMATION: oligonucleotide probe
<400> SEQUENCE: 108
agcaccaccg acgatgacat cgcac
#
# 25
<210> SEQ ID NO 109
<211> LENGTH: 25
<212> TYPE: DNA
<213> ORGANISM: Artificial sequence
<220> FEATURE:
<223> OTHER INFORMATION: oligonucleotide probe
<400> SEQUENCE: 109
caccgacaat gacatcgcac tgctg
#
# 25
<210> SEQ ID NO 110
<211> LENGTH: 25
<212> TYPE: DNA
<213> ORGANISM: Artificial sequence
<220> FEATURE:
<223> OTHER INFORMATION: oligonucleotide probe
<400> SEQUENCE: 110
ccaccgacaa tgatcgcact gctgc
#
# 25
<210> SEQ ID NO 111
<211> LENGTH: 25
<212> TYPE: DNA
<213> ORGANISM: Artificial sequence
<220> FEATURE:
<223> OTHER INFORMATION: oligonucleotide probe
<400> SEQUENCE: 111
ccatctgcct cccggacagc ggcct
#
# 25
<210> SEQ ID NO 112
<211> LENGTH: 25
<212> TYPE: DNA
<213> ORGANISM: Artificial sequence
<220> FEATURE:
<223> OTHER INFORMATION: oligonucleotide probe
<400> SEQUENCE: 112
ccatctgcct cctggacagc ggcct
#
# 25
<210> SEQ ID NO 113
<211> LENGTH: 25
<212> TYPE: DNA
<213> ORGANISM: Artificial sequence
<220> FEATURE:
<223> OTHER INFORMATION: oligonucleotide probe
<400> SEQUENCE: 113
ctcccggaca gcggccttgc agagc
#
# 25
<210> SEQ ID NO 114
<211> LENGTH: 25
<212> TYPE: DNA
<213> ORGANISM: Artificial sequence
<220> FEATURE:
<223> OTHER INFORMATION: oligonucleotide probe
<400> SEQUENCE: 114
ctcccggaca gcagccttgc agagc
#
# 25
<210> SEQ ID NO 115
<211> LENGTH: 25
<212> TYPE: DNA
<213> ORGANISM: Artificial sequence
<220> FEATURE:
<223> OTHER INFORMATION: oligonucleotide probe
<400> SEQUENCE: 115
ggccttgcag agcgcgagct caatc
#
# 25
<210> SEQ ID NO 116
<211> LENGTH: 25
<212> TYPE: DNA
<213> ORGANISM: Artificial sequence
<220> FEATURE:
<223> OTHER INFORMATION: oligonucleotide probe
<400> SEQUENCE: 116
ggccttgcag agtgcgagct caatc
#
# 25
<210> SEQ ID NO 117
<211> LENGTH: 25
<212> TYPE: DNA
<213> ORGANISM: Artificial sequence
<220> FEATURE:
<223> OTHER INFORMATION: oligonucleotide probe
<400> SEQUENCE: 117
gccttgcaga gcgcgagctc aatca
#
# 25
<210> SEQ ID NO 118
<211> LENGTH: 25
<212> TYPE: DNA
<213> ORGANISM: Artificial sequence
<220> FEATURE:
<223> OTHER INFORMATION: oligonucleotide probe
<400> SEQUENCE: 118
gccttgcaga gcacgagctc aatca
#
# 25
<210> SEQ ID NO 119
<211> LENGTH: 25
<212> TYPE: DNA
<213> ORGANISM: Artificial sequence
<220> FEATURE:
<223> OTHER INFORMATION: oligonucleotide probe
<400> SEQUENCE: 119
ctcaatcagg ccggccagga gaccc
#
# 25
<210> SEQ ID NO 120
<211> LENGTH: 25
<212> TYPE: DNA
<213> ORGANISM: Artificial sequence
<220> FEATURE:
<223> OTHER INFORMATION: oligonucleotide probe
<400> SEQUENCE: 120
ctcaatcagg ccagccagga gaccc
#
# 25
<210> SEQ ID NO 121
<211> LENGTH: 25
<212> TYPE: DNA
<213> ORGANISM: Artificial sequence
<220> FEATURE:
<223> OTHER INFORMATION: oligonucleotide probe
<400> SEQUENCE: 121
caggagaccc tcgtgacggg ctggg
#
# 25
<210> SEQ ID NO 122
<211> LENGTH: 25
<212> TYPE: DNA
<213> ORGANISM: Artificial sequence
<220> FEATURE:
<223> OTHER INFORMATION: oligonucleotide probe
<400> SEQUENCE: 122
caggagaccc tcatgacggg ctggg
#
# 25
<210> SEQ ID NO 123
<211> LENGTH: 25
<212> TYPE: DNA
<213> ORGANISM: Artificial sequence
<220> FEATURE:
<223> OTHER INFORMATION: oligonucleotide probe
<400> SEQUENCE: 123
agaccctcgt gacgggctgg ggcta
#
# 25
<210> SEQ ID NO 124
<211> LENGTH: 25
<212> TYPE: DNA
<213> ORGANISM: Artificial sequence
<220> FEATURE:
<223> OTHER INFORMATION: oligonucleotide probe
<400> SEQUENCE: 124
agaccctcgt gatgggctgg ggcta
#
# 25
<210> SEQ ID NO 125
<211> LENGTH: 25
<212> TYPE: DNA
<213> ORGANISM: Artificial sequence
<220> FEATURE:
<223> OTHER INFORMATION: oligonucleotide probe
<400> SEQUENCE: 125
taccacagca gccgagagaa ggagg
#
# 25
<210> SEQ ID NO 126
<211> LENGTH: 25
<212> TYPE: DNA
<213> ORGANISM: Artificial sequence
<220> FEATURE:
<223> OTHER INFORMATION: oligonucleotide probe
<400> SEQUENCE: 126
taccacagca gctgagagaa ggagg
#
# 25
<210> SEQ ID NO 127
<211> LENGTH: 25
<212> TYPE: DNA
<213> ORGANISM: Artificial sequence
<220> FEATURE:
<223> OTHER INFORMATION: oligonucleotide probe
<400> SEQUENCE: 127
ctcaacttca tcaagattcc cgtgg
#
# 25
<210> SEQ ID NO 128
<211> LENGTH: 25
<212> TYPE: DNA
<213> ORGANISM: Artificial sequence
<220> FEATURE:
<223> OTHER INFORMATION: oligonucleotide probe
<400> SEQUENCE: 128
cctcaacttc atgattcccg tggtc
#
# 25
<210> SEQ ID NO 129
<211> LENGTH: 25
<212> TYPE: DNA
<213> ORGANISM: Artificial sequence
<220> FEATURE:
<223> OTHER INFORMATION: oligonucleotide probe
<400> SEQUENCE: 129
tcaagattcc cgtggtcccg cacaa
#
# 25
<210> SEQ ID NO 130
<211> LENGTH: 25
<212> TYPE: DNA
<213> ORGANISM: Artificial sequence
<220> FEATURE:
<223> OTHER INFORMATION: oligonucleotide probe
<400> SEQUENCE: 130
tcaagattcc cgcggtcccg cacaa
#
# 25
<210> SEQ ID NO 131
<211> LENGTH: 25
<212> TYPE: DNA
<213> ORGANISM: Artificial sequence
<220> FEATURE:
<223> OTHER INFORMATION: oligonucleotide probe
<400> SEQUENCE: 131
ttcccgtggt cccgcacaat gagtg
#
# 25
<210> SEQ ID NO 132
<211> LENGTH: 25
<212> TYPE: DNA
<213> ORGANISM: Artificial sequence
<220> FEATURE:
<223> OTHER INFORMATION: oligonucleotide probe
<400> SEQUENCE: 132
ttcccgtggt cctgcacaat gagtg
#
# 25
<210> SEQ ID NO 133
<211> LENGTH: 25
<212> TYPE: DNA
<213> ORGANISM: Artificial sequence
<220> FEATURE:
<223> OTHER INFORMATION: oligonucleotide probe
<400> SEQUENCE: 133
gcgggcatcc tcggggaccg gcagg
#
# 25
<210> SEQ ID NO 134
<211> LENGTH: 25
<212> TYPE: DNA
<213> ORGANISM: Artificial sequence
<220> FEATURE:
<223> OTHER INFORMATION: oligonucleotide probe
<400> SEQUENCE: 134
gcgggcatcc tcagggaccg gcagg
#
# 25
<210> SEQ ID NO 135
<211> LENGTH: 25
<212> TYPE: DNA
<213> ORGANISM: Artificial sequence
<220> FEATURE:
<223> OTHER INFORMATION: oligonucleotide probe
<400> SEQUENCE: 135
tggtgagctg gggtgagggc tgtgg
#
# 25
<210> SEQ ID NO 136
<211> LENGTH: 25
<212> TYPE: DNA
<213> ORGANISM: Artificial sequence
<220> FEATURE:
<223> OTHER INFORMATION: oligonucleotide probe
<400> SEQUENCE: 136
tggtgagctg ggtgagggct gtggg
#
# 25
<210> SEQ ID NO 137
<211> LENGTH: 25
<212> TYPE: DNA
<213> ORGANISM: Artificial sequence
<220> FEATURE:
<223> OTHER INFORMATION: oligonucleotide probe
<400> SEQUENCE: 137
tacggcgttt acaccaaagt cagcc
#
# 25
<210> SEQ ID NO 138
<211> LENGTH: 25
<212> TYPE: DNA
<213> ORGANISM: Artificial sequence
<220> FEATURE:
<223> OTHER INFORMATION: oligonucleotide probe
<400> SEQUENCE: 138
tacggcgttt accccaaagt cagcc
#
# 25
<210> SEQ ID NO 139
<211> LENGTH: 25
<212> TYPE: DNA
<213> ORGANISM: Artificial sequence
<220> FEATURE:
<223> OTHER INFORMATION: oligonucleotide probe
<400> SEQUENCE: 139
cctcgactgg atccatgggc acatc
#
# 25
<210> SEQ ID NO 140
<211> LENGTH: 25
<212> TYPE: DNA
<213> ORGANISM: Artificial sequence
<220> FEATURE:
<223> OTHER INFORMATION: oligonucleotide probe
<400> SEQUENCE: 140
cctcgactgg atgcatgggc acatc
#
# 25
<210> SEQ ID NO 141
<211> LENGTH: 25
<212> TYPE: DNA
<213> ORGANISM: Artificial sequence
<220> FEATURE:
<223> OTHER INFORMATION: oligonucleotide probe
<400> SEQUENCE: 141
cggatccgca aacgtgccaa ctcct
#
# 25
<210> SEQ ID NO 142
<211> LENGTH: 25
<212> TYPE: DNA
<213> ORGANISM: Artificial sequence
<220> FEATURE:
<223> OTHER INFORMATION: oligonucleotide probe
<400> SEQUENCE: 142
cggatccgca aatgtgccaa ctcct
#
# 25
<210> SEQ ID NO 143
<211> LENGTH: 25
<212> TYPE: DNA
<213> ORGANISM: Artificial sequence
<220> FEATURE:
<223> OTHER INFORMATION: oligonucleotide probe
<400> SEQUENCE: 143
ggatccgcaa acgtgccaac tcctt
#
# 25
<210> SEQ ID NO 144
<211> LENGTH: 25
<212> TYPE: DNA
<213> ORGANISM: Artificial sequence
<220> FEATURE:
<223> OTHER INFORMATION: oligonucleotide probe
<400> SEQUENCE: 144
ggatccgcaa acatgccaac tcctt
#
# 25
<210> SEQ ID NO 145
<211> LENGTH: 25
<212> TYPE: DNA
<213> ORGANISM: Artificial sequence
<220> FEATURE:
<223> OTHER INFORMATION: oligonucleotide probe
<400> SEQUENCE: 145
agcagcctgg agcgggagtg catag
#
# 25
<210> SEQ ID NO 146
<211> LENGTH: 25
<212> TYPE: DNA
<213> ORGANISM: Artificial sequence
<220> FEATURE:
<223> OTHER INFORMATION: oligonucleotide probe
<400> SEQUENCE: 146
agcagcctgg agtgggagtg catag
#
# 25
<210> SEQ ID NO 147
<211> LENGTH: 25
<212> TYPE: DNA
<213> ORGANISM: Artificial sequence
<220> FEATURE:
<223> OTHER INFORMATION: oligonucleotide probe
<400> SEQUENCE: 147
caagtagatc cgcggctcat tgatg
#
# 25
<210> SEQ ID NO 148
<211> LENGTH: 25
<212> TYPE: DNA
<213> ORGANISM: Artificial sequence
<220> FEATURE:
<223> OTHER INFORMATION: oligonucleotide probe
<400> SEQUENCE: 148
caagtagatc cgtggctcat tgatg
#
# 25
<210> SEQ ID NO 149
<211> LENGTH: 25
<212> TYPE: DNA
<213> ORGANISM: Artificial sequence
<220> FEATURE:
<223> OTHER INFORMATION: oligonucleotide probe
<400> SEQUENCE: 149
aagtagatcc gcggctcatt gatgg
#
# 25
<210> SEQ ID NO 150
<211> LENGTH: 25
<212> TYPE: DNA
<213> ORGANISM: Artificial sequence
<220> FEATURE:
<223> OTHER INFORMATION: oligonucleotide probe
<400> SEQUENCE: 150
aagtagatcc gcagctcatt gatgg
#
# 25
<210> SEQ ID NO 151
<211> LENGTH: 25
<212> TYPE: DNA
<213> ORGANISM: Artificial sequence
<220> FEATURE:
<223> OTHER INFORMATION: oligonucleotide probe
<400> SEQUENCE: 151
gacagcggcc cactgcatgg atgag
#
# 25
<210> SEQ ID NO 152
<211> LENGTH: 25
<212> TYPE: DNA
<213> ORGANISM: Artificial sequence
<220> FEATURE:
<223> OTHER INFORMATION: oligonucleotide probe
<400> SEQUENCE: 152
gacagcggcc caatgcatgg atgag
#
# 25
<210> SEQ ID NO 153
<211> LENGTH: 25
<212> TYPE: DNA
<213> ORGANISM: Artificial sequence
<220> FEATURE:
<223> OTHER INFORMATION: oligonucleotide probe
<400> SEQUENCE: 153
actacagcaa gagcaccacc gacaa
#
# 25
<210> SEQ ID NO 154
<211> LENGTH: 25
<212> TYPE: DNA
<213> ORGANISM: Artificial sequence
<220> FEATURE:
<223> OTHER INFORMATION: oligonucleotide probe
<400> SEQUENCE: 154
actacagcaa gaacaccacc gacaa
#
# 25
<210> SEQ ID NO 155
<211> LENGTH: 25
<212> TYPE: DNA
<213> ORGANISM: Artificial sequence
<220> FEATURE:
<223> OTHER INFORMATION: oligonucleotide probe
<400> SEQUENCE: 155
gtctgagaac atgctgtgtg cgggc
#
# 25
<210> SEQ ID NO 156
<211> LENGTH: 25
<212> TYPE: DNA
<213> ORGANISM: Artificial sequence
<220> FEATURE:
<223> OTHER INFORMATION: oligonucleotide probe
<400> SEQUENCE: 156
gtctgagaac atactgtgtg cgggc
#
# 25
<210> SEQ ID NO 157
<211> LENGTH: 25
<212> TYPE: DNA
<213> ORGANISM: Artificial sequence
<220> FEATURE:
<223> OTHER INFORMATION: oligonucleotide probe
<400> SEQUENCE: 157
atcctcgggg accggcagga tgcct
#
# 25
<210> SEQ ID NO 158
<211> LENGTH: 25
<212> TYPE: DNA
<213> ORGANISM: Artificial sequence
<220> FEATURE:
<223> OTHER INFORMATION: oligonucleotide probe
<400> SEQUENCE: 158
atcctcgggg actggcagga tgcct
#
# 25
<210> SEQ ID NO 159
<211> LENGTH: 25
<212> TYPE: DNA
<213> ORGANISM: Artificial sequence
<220> FEATURE:
<223> OTHER INFORMATION: oligonucleotide probe
<400> SEQUENCE: 159
gcctgcgagg gcgacagtgg ggggc
#
# 25
<210> SEQ ID NO 160
<211> LENGTH: 25
<212> TYPE: DNA
<213> ORGANISM: Artificial sequence
<220> FEATURE:
<223> OTHER INFORMATION: oligonucleotide probe
<400> SEQUENCE: 160
gcctgcgagg gcaacagtgg ggggc
#
# 25
<210> SEQ ID NO 161
<211> LENGTH: 25
<212> TYPE: DNA
<213> ORGANISM: Artificial sequence
<220> FEATURE:
<223> OTHER INFORMATION: oligonucleotide probe
<400> SEQUENCE: 161
cttcacaact acggcgttta cacca
#
# 25
<210> SEQ ID NO 162
<211> LENGTH: 25
<212> TYPE: DNA
<213> ORGANISM: Artificial sequence
<220> FEATURE:
<223> OTHER INFORMATION: oligonucleotide probe
<400> SEQUENCE: 162
cttcacaact acagcgttta cacca
#
# 25
<210> SEQ ID NO 163
<211> LENGTH: 25
<212> TYPE: DNA
<213> ORGANISM: Artificial sequence
<220> FEATURE:
<223> OTHER INFORMATION: oligonucleotide probe
<400> SEQUENCE: 163
ctccctgctg gatggcatcc ttggt
#
# 25
<210> SEQ ID NO 164
<211> LENGTH: 25
<212> TYPE: DNA
<213> ORGANISM: Artificial sequence
<220> FEATURE:
<223> OTHER INFORMATION: oligonucleotide probe
<400> SEQUENCE: 164
ctccctgctg gacggcatcc ttggt
#
# 25
<210> SEQ ID NO 165
<211> LENGTH: 25
<212> TYPE: DNA
<213> ORGANISM: Artificial sequence
<220> FEATURE:
<223> OTHER INFORMATION: oligonucleotide probe
<400> SEQUENCE: 165
ggcatccttg gtaggcagag gtggg
#
# 25
<210> SEQ ID NO 166
<211> LENGTH: 25
<212> TYPE: DNA
<213> ORGANISM: Artificial sequence
<220> FEATURE:
<223> OTHER INFORMATION: oligonucleotide probe
<400> SEQUENCE: 166
ggcatccttg gtgggcagag gtggg
#
# 25
<210> SEQ ID NO 167
<211> LENGTH: 25
<212> TYPE: DNA
<213> ORGANISM: Artificial sequence
<220> FEATURE:
<223> OTHER INFORMATION: oligonucleotide probe
<400> SEQUENCE: 167
ggcaggacgg cgaacttgca gtatc
#
# 25
<210> SEQ ID NO 168
<211> LENGTH: 25
<212> TYPE: DNA
<213> ORGANISM: Artificial sequence
<220> FEATURE:
<223> OTHER INFORMATION: oligonucleotide probe
<400> SEQUENCE: 168
ggcaggacgg cgtacttgca gtatc
#
# 25
<210> SEQ ID NO 169
<211> LENGTH: 25
<212> TYPE: DNA
<213> ORGANISM: Artificial sequence
<220> FEATURE:
<223> OTHER INFORMATION: oligonucleotide probe
<400> SEQUENCE: 169
tgggtgggcg ctgcggggcg ctgct
#
# 25
<210> SEQ ID NO 170
<211> LENGTH: 25
<212> TYPE: DNA
<213> ORGANISM: Artificial sequence
<220> FEATURE:
<223> OTHER INFORMATION: oligonucleotide probe
<400> SEQUENCE: 170
tgggtgggcg ctcggggcgc tgctg
#
# 25
<210> SEQ ID NO 171
<211> LENGTH: 25
<212> TYPE: DNA
<213> ORGANISM: Artificial sequence
<220> FEATURE:
<223> OTHER INFORMATION: oligonucleotide probe
<400> SEQUENCE: 171
gtctcctcct agtgcttccc gtctc
#
# 25
<210> SEQ ID NO 172
<211> LENGTH: 25
<212> TYPE: DNA
<213> ORGANISM: Artificial sequence
<220> FEATURE:
<223> OTHER INFORMATION: oligonucleotide probe
<400> SEQUENCE: 172
gtctcctcct agagcttccc gtctc
#
# 25
<210> SEQ ID NO 173
<211> LENGTH: 25
<212> TYPE: DNA
<213> ORGANISM: Artificial sequence
<220> FEATURE:
<223> OTHER INFORMATION: oligonucleotide probe
<400> SEQUENCE: 173
agagaatgca tcgaagaact gtgca
#
# 25
<210> SEQ ID NO 174
<211> LENGTH: 25
<212> TYPE: DNA
<213> ORGANISM: Artificial sequence
<220> FEATURE:
<223> OTHER INFORMATION: oligonucleotide probe
<400> SEQUENCE: 174
agagaatgca tctaagaact gtgca
#
# 25
<210> SEQ ID NO 175
<211> LENGTH: 25
<212> TYPE: DNA
<213> ORGANISM: Artificial sequence
<220> FEATURE:
<223> OTHER INFORMATION: oligonucleotide probe
<400> SEQUENCE: 175
gcaataaaga agaagccagg gaggt
#
# 25
<210> SEQ ID NO 176
<211> LENGTH: 25
<212> TYPE: DNA
<213> ORGANISM: Artificial sequence
<220> FEATURE:
<223> OTHER INFORMATION: oligonucleotide probe
<400> SEQUENCE: 176
gcaataaaga agcagccagg gaggt
#
# 25
<210> SEQ ID NO 177
<211> LENGTH: 25
<212> TYPE: DNA
<213> ORGANISM: Artificial sequence
<220> FEATURE:
<223> OTHER INFORMATION: oligonucleotide probe
<400> SEQUENCE: 177
atccaaaata cttaggtaag ttcaa
#
# 25
<210> SEQ ID NO 178
<211> LENGTH: 25
<212> TYPE: DNA
<213> ORGANISM: Artificial sequence
<220> FEATURE:
<223> OTHER INFORMATION: oligonucleotide probe
<400> SEQUENCE: 178
ttatccaaaa taggtaagtt caaaa
#
# 25
<210> SEQ ID NO 179
<211> LENGTH: 25
<212> TYPE: DNA
<213> ORGANISM: Artificial sequence
<220> FEATURE:
<223> OTHER INFORMATION: oligonucleotide probe
<400> SEQUENCE: 179
ccaaaatact taggtaagtt caaaa
#
# 25
<210> SEQ ID NO 180
<211> LENGTH: 25
<212> TYPE: DNA
<213> ORGANISM: Artificial sequence
<220> FEATURE:
<223> OTHER INFORMATION: oligonucleotide probe
<400> SEQUENCE: 180
ccaaaatact tacgtaagtt caaaa
#
# 25
<210> SEQ ID NO 181
<211> LENGTH: 25
<212> TYPE: DNA
<213> ORGANISM: Artificial sequence
<220> FEATURE:
<223> OTHER INFORMATION: oligonucleotide probe
<400> SEQUENCE: 181
gctgtgtcaa tggtaagcac ttcta
#
# 25
<210> SEQ ID NO 182
<211> LENGTH: 25
<212> TYPE: DNA
<213> ORGANISM: Artificial sequence
<220> FEATURE:
<223> OTHER INFORMATION: oligonucleotide probe
<400> SEQUENCE: 182
gctgtgtcaa tgataagcac ttcta
#
# 25
<210> SEQ ID NO 183
<211> LENGTH: 25
<212> TYPE: DNA
<213> ORGANISM: Artificial sequence
<220> FEATURE:
<223> OTHER INFORMATION: oligonucleotide probe
<400> SEQUENCE: 183
atgctttcaa ataagaaaga ttgta
#
# 25
<210> SEQ ID NO 184
<211> LENGTH: 25
<212> TYPE: DNA
<213> ORGANISM: Artificial sequence
<220> FEATURE:
<223> OTHER INFORMATION: oligonucleotide probe
<400> SEQUENCE: 184
atgctttcaa atgagaaaga ttgta
#
# 25
<210> SEQ ID NO 185
<211> LENGTH: 25
<212> TYPE: DNA
<213> ORGANISM: Artificial sequence
<220> FEATURE:
<223> OTHER INFORMATION: oligonucleotide probe
<400> SEQUENCE: 185
agctttgtgt caattaccct ggagg
#
# 25
<210> SEQ ID NO 186
<211> LENGTH: 25
<212> TYPE: DNA
<213> ORGANISM: Artificial sequence
<220> FEATURE:
<223> OTHER INFORMATION: oligonucleotide probe
<400> SEQUENCE: 186
agctttgtgt cagttaccct ggagg
#
# 25
<210> SEQ ID NO 187
<211> LENGTH: 25
<212> TYPE: DNA
<213> ORGANISM: Artificial sequence
<220> FEATURE:
<223> OTHER INFORMATION: oligonucleotide probe
<400> SEQUENCE: 187
cttgcccaag atcagaagag ttgtg
#
# 25
<210> SEQ ID NO 188
<211> LENGTH: 25
<212> TYPE: DNA
<213> ORGANISM: Artificial sequence
<220> FEATURE:
<223> OTHER INFORMATION: oligonucleotide probe
<400> SEQUENCE: 188
cttgcccaag attagaagag ttgtg
#
# 25
<210> SEQ ID NO 189
<211> LENGTH: 25
<212> TYPE: DNA
<213> ORGANISM: Artificial sequence
<220> FEATURE:
<223> OTHER INFORMATION: oligonucleotide probe
<400> SEQUENCE: 189
tggcggagca gtttgcaggg gttgt
#
# 25
<210> SEQ ID NO 190
<211> LENGTH: 25
<212> TYPE: DNA
<213> ORGANISM: Artificial sequence
<220> FEATURE:
<223> OTHER INFORMATION: oligonucleotide probe
<400> SEQUENCE: 190
ggcggagcag ttttgcaggg gttgt
#
# 25
<210> SEQ ID NO 191
<211> LENGTH: 25
<212> TYPE: DNA
<213> ORGANISM: Artificial sequence
<220> FEATURE:
<223> OTHER INFORMATION: oligonucleotide probe
<400> SEQUENCE: 191
ggacatatga ttcagaaggc gtgat
#
# 25
<210> SEQ ID NO 192
<211> LENGTH: 25
<212> TYPE: DNA
<213> ORGANISM: Artificial sequence
<220> FEATURE:
<223> OTHER INFORMATION: oligonucleotide probe
<400> SEQUENCE: 192
ggacatatga ttgagaaggc gtgat
#
# 25
<210> SEQ ID NO 193
<211> LENGTH: 25
<212> TYPE: DNA
<213> ORGANISM: Artificial sequence
<220> FEATURE:
<223> OTHER INFORMATION: oligonucleotide probe
<400> SEQUENCE: 193
atgattcaga aggcgtgata ctgta
#
# 25
<210> SEQ ID NO 194
<211> LENGTH: 25
<212> TYPE: DNA
<213> ORGANISM: Artificial sequence
<220> FEATURE:
<223> OTHER INFORMATION: oligonucleotide probe
<400> SEQUENCE: 194
atgattcaga agtcgtgata ctgta
#
# 25
<210> SEQ ID NO 195
<211> LENGTH: 25
<212> TYPE: DNA
<213> ORGANISM: Artificial sequence
<220> FEATURE:
<223> OTHER INFORMATION: oligonucleotide probe
<400> SEQUENCE: 195
ggaatatggt acgtttgcag atttc
#
# 25
<210> SEQ ID NO 196
<211> LENGTH: 25
<212> TYPE: DNA
<213> ORGANISM: Artificial sequence
<220> FEATURE:
<223> OTHER INFORMATION: oligonucleotide probe
<400> SEQUENCE: 196
ggaatatggt acatttgcag atttc
#
# 25
<210> SEQ ID NO 197
<211> LENGTH: 25
<212> TYPE: DNA
<213> ORGANISM: Artificial sequence
<220> FEATURE:
<223> OTHER INFORMATION: oligonucleotide probe
<400> SEQUENCE: 197
gtgtctgtgg aagaattaga acata
#
# 25
<210> SEQ ID NO 198
<211> LENGTH: 25
<212> TYPE: DNA
<213> ORGANISM: Artificial sequence
<220> FEATURE:
<223> OTHER INFORMATION: oligonucleotide probe
<400> SEQUENCE: 198
gtgtctgtgg aaaaattaga acata
#
# 25
<210> SEQ ID NO 199
<211> LENGTH: 25
<212> TYPE: DNA
<213> ORGANISM: Artificial sequence
<220> FEATURE:
<223> OTHER INFORMATION: oligonucleotide probe
<400> SEQUENCE: 199
acctggaccc ctttttaagc cggaa
#
# 25
<210> SEQ ID NO 200
<211> LENGTH: 25
<212> TYPE: DNA
<213> ORGANISM: Artificial sequence
<220> FEATURE:
<223> OTHER INFORMATION: oligonucleotide probe
<400> SEQUENCE: 200
aacctggacc caaaagccgg aaaat
#
# 25
<210> SEQ ID NO 201
<211> LENGTH: 25
<212> TYPE: DNA
<213> ORGANISM: Artificial sequence
<220> FEATURE:
<223> OTHER INFORMATION: oligonucleotide probe
<400> SEQUENCE: 201
acttgtattt taatttgtta gatta
#
# 25
<210> SEQ ID NO 202
<211> LENGTH: 25
<212> TYPE: DNA
<213> ORGANISM: Artificial sequence
<220> FEATURE:
<223> OTHER INFORMATION: oligonucleotide probe
<400> SEQUENCE: 202
acttgtattt tagtttgtta gatta
#
# 25
<210> SEQ ID NO 203
<211> LENGTH: 25
<212> TYPE: DNA
<213> ORGANISM: Artificial sequence
<220> FEATURE:
<223> OTHER INFORMATION: oligonucleotide probe
<400> SEQUENCE: 203
ttaaccctcg tctagatgga tgtat
#
# 25
<210> SEQ ID NO 204
<211> LENGTH: 25
<212> TYPE: DNA
<213> ORGANISM: Artificial sequence
<220> FEATURE:
<223> OTHER INFORMATION: oligonucleotide probe
<400> SEQUENCE: 204
ttaaccctcg tccagatgga tgtat
#
# 25
<210> SEQ ID NO 205
<211> LENGTH: 25
<212> TYPE: DNA
<213> ORGANISM: Artificial sequence
<220> FEATURE:
<223> OTHER INFORMATION: oligonucleotide probe
<400> SEQUENCE: 205
gatggatgta tacgaagctg gaatt
#
# 25
<210> SEQ ID NO 206
<211> LENGTH: 25
<212> TYPE: DNA
<213> ORGANISM: Artificial sequence
<220> FEATURE:
<223> OTHER INFORMATION: oligonucleotide probe
<400> SEQUENCE: 206
gatggatgta tatgaagctg gaatt
#
# 25
<210> SEQ ID NO 207
<211> LENGTH: 25
<212> TYPE: DNA
<213> ORGANISM: Artificial sequence
<220> FEATURE:
<223> OTHER INFORMATION: oligonucleotide probe
<400> SEQUENCE: 207
aagaaaaaca aaataagcat tgcct
#
# 25
<210> SEQ ID NO 208
<211> LENGTH: 25
<212> TYPE: DNA
<213> ORGANISM: Artificial sequence
<220> FEATURE:
<223> OTHER INFORMATION: oligonucleotide probe
<400> SEQUENCE: 208
aagaaaaaca aaaataagca ttgcc
#
# 25
<210> SEQ ID NO 209
<211> LENGTH: 25
<212> TYPE: DNA
<213> ORGANISM: Artificial sequence
<220> FEATURE:
<223> OTHER INFORMATION: oligonucleotide probe
<400> SEQUENCE: 209
tgctgagggt tggcatgtaa atgtg
#
# 25
<210> SEQ ID NO 210
<211> LENGTH: 25
<212> TYPE: DNA
<213> ORGANISM: Artificial sequence
<220> FEATURE:
<223> OTHER INFORMATION: oligonucleotide probe
<400> SEQUENCE: 210
tgctgagggt tgacatgtaa atgtg
#
# 25
<210> SEQ ID NO 211
<211> LENGTH: 25
<212> TYPE: DNA
<213> ORGANISM: Artificial sequence
<220> FEATURE:
<223> OTHER INFORMATION: oligonucleotide probe
<400> SEQUENCE: 211
accttgaata ttcgtccatc cacgg
#
# 25
<210> SEQ ID NO 212
<211> LENGTH: 25
<212> TYPE: DNA
<213> ORGANISM: Artificial sequence
<220> FEATURE:
<223> OTHER INFORMATION: oligonucleotide probe
<400> SEQUENCE: 212
accttgaata tttgtccatc cacgg
#
# 25
<210> SEQ ID NO 213
<211> LENGTH: 25
<212> TYPE: DNA
<213> ORGANISM: Artificial sequence
<220> FEATURE:
<223> OTHER INFORMATION: oligonucleotide probe
<400> SEQUENCE: 213
atatatcgga tacaggccct aagtc
#
# 25
<210> SEQ ID NO 214
<211> LENGTH: 25
<212> TYPE: DNA
<213> ORGANISM: Artificial sequence
<220> FEATURE:
<223> OTHER INFORMATION: oligonucleotide probe
<400> SEQUENCE: 214
atatatcgga tataggccct aagtc
#
# 25
<210> SEQ ID NO 215
<211> LENGTH: 25
<212> TYPE: DNA
<213> ORGANISM: Artificial sequence
<220> FEATURE:
<223> OTHER INFORMATION: oligonucleotide probe
<400> SEQUENCE: 215
aacaatctca tctggaattt agagt
#
# 25
<210> SEQ ID NO 216
<211> LENGTH: 25
<212> TYPE: DNA
<213> ORGANISM: Artificial sequence
<220> FEATURE:
<223> OTHER INFORMATION: oligonucleotide probe
<400> SEQUENCE: 216
aacaatctca tcgggaattt agagt
#
# 25
<210> SEQ ID NO 217
<211> LENGTH: 25
<212> TYPE: DNA
<213> ORGANISM: Artificial sequence
<220> FEATURE:
<223> OTHER INFORMATION: oligonucleotide probe
<400> SEQUENCE: 217
agagctcact catgtccatc agttt
#
# 25
<210> SEQ ID NO 218
<211> LENGTH: 25
<212> TYPE: DNA
<213> ORGANISM: Artificial sequence
<220> FEATURE:
<223> OTHER INFORMATION: oligonucleotide probe
<400> SEQUENCE: 218
agagctcact cacgtccatc agttt
#
# 25
<210> SEQ ID NO 219
<211> LENGTH: 25
<212> TYPE: DNA
<213> ORGANISM: Artificial sequence
<220> FEATURE:
<223> OTHER INFORMATION: oligonucleotide probe
<400> SEQUENCE: 219
tggttaggaa gcgtcgtgca aattc
#
# 25
<210> SEQ ID NO 220
<211> LENGTH: 25
<212> TYPE: DNA
<213> ORGANISM: Artificial sequence
<220> FEATURE:
<223> OTHER INFORMATION: oligonucleotide probe
<400> SEQUENCE: 220
tggttaggaa gcttcgtgca aattc
#
# 25
<210> SEQ ID NO 221
<211> LENGTH: 25
<212> TYPE: DNA
<213> ORGANISM: Artificial sequence
<220> FEATURE:
<223> OTHER INFORMATION: oligonucleotide probe
<400> SEQUENCE: 221
cttgaagaaa ccaaacaggg taatc
#
# 25
<210> SEQ ID NO 222
<211> LENGTH: 25
<212> TYPE: DNA
<213> ORGANISM: Artificial sequence
<220> FEATURE:
<223> OTHER INFORMATION: oligonucleotide probe
<400> SEQUENCE: 222
cttgaagaaa ccgaacaggg taatc
#
# 25
<210> SEQ ID NO 223
<211> LENGTH: 25
<212> TYPE: DNA
<213> ORGANISM: Artificial sequence
<220> FEATURE:
<223> OTHER INFORMATION: oligonucleotide probe
<400> SEQUENCE: 223
tgaatttggt acgtataata acccc
#
# 25
<210> SEQ ID NO 224
<211> LENGTH: 25
<212> TYPE: DNA
<213> ORGANISM: Artificial sequence
<220> FEATURE:
<223> OTHER INFORMATION: oligonucleotide probe
<400> SEQUENCE: 224
tgaatttggt acatataata acccc
#
# 25
<210> SEQ ID NO 225
<211> LENGTH: 25
<212> TYPE: DNA
<213> ORGANISM: Artificial sequence
<220> FEATURE:
<223> OTHER INFORMATION: oligonucleotide probe
<400> SEQUENCE: 225
cgtgtctcct cctagtgctt cccgt
#
# 25
<210> SEQ ID NO 226
<211> LENGTH: 25
<212> TYPE: DNA
<213> ORGANISM: Artificial sequence
<220> FEATURE:
<223> OTHER INFORMATION: oligonucleotide probe
<400> SEQUENCE: 226
cgtgtctcct ccttagtgct tccgt
#
# 25
<210> SEQ ID NO 227
<211> LENGTH: 25
<212> TYPE: DNA
<213> ORGANISM: Artificial sequence
<220> FEATURE:
<223> OTHER INFORMATION: oligonucleotide probe
<400> SEQUENCE: 227
agggttggca tgtaaatgtg acctt
#
# 25
<210> SEQ ID NO 228
<211> LENGTH: 25
<212> TYPE: DNA
<213> ORGANISM: Artificial sequence
<220> FEATURE:
<223> OTHER INFORMATION: oligonucleotide probe
<400> SEQUENCE: 228
agggttggca tggaaatgtg acctt
#
# 25
<210> SEQ ID NO 229
<211> LENGTH: 25
<212> TYPE: DNA
<213> ORGANISM: Artificial sequence
<220> FEATURE:
<223> OTHER INFORMATION: oligonucleotide probe
<400> SEQUENCE: 229
tggaaaaaga caaagaattc ttaag
#
# 25
<210> SEQ ID NO 230
<211> LENGTH: 25
<212> TYPE: DNA
<213> ORGANISM: Artificial sequence
<220> FEATURE:
<223> OTHER INFORMATION: oligonucleotide probe
<400> SEQUENCE: 230
ttggaaaaag acagaattct taagg
#
# 25
<210> SEQ ID NO 231
<211> LENGTH: 25
<212> TYPE: DNA
<213> ORGANISM: Artificial sequence
<220> FEATURE:
<223> OTHER INFORMATION: oligonucleotide probe
<400> SEQUENCE: 231
aaagaattct taaggcatct tttct
#
# 25
<210> SEQ ID NO 232
<211> LENGTH: 25
<212> TYPE: DNA
<213> ORGANISM: Artificial sequence
<220> FEATURE:
<223> OTHER INFORMATION: oligonucleotide probe
<400> SEQUENCE: 232
aaagaattct tatggcatct tttct
#
# 25
<210> SEQ ID NO 233
<211> LENGTH: 25
<212> TYPE: DNA
<213> ORGANISM: Artificial sequence
<220> FEATURE:
<223> OTHER INFORMATION: oligonucleotide probe
<400> SEQUENCE: 233
ttttgtctgt aacagatttg aatat
#
# 25
<210> SEQ ID NO 234
<211> LENGTH: 25
<212> TYPE: DNA
<213> ORGANISM: Artificial sequence
<220> FEATURE:
<223> OTHER INFORMATION: oligonucleotide probe
<400> SEQUENCE: 234
ttttgtctgt aatagatttg aatat
#
# 25
<210> SEQ ID NO 235
<211> LENGTH: 25
<212> TYPE: DNA
<213> ORGANISM: Artificial sequence
<220> FEATURE:
<223> OTHER INFORMATION: oligonucleotide probe
<400> SEQUENCE: 235
atagataatg tatccagtgc tgagg
#
# 25
<210> SEQ ID NO 236
<211> LENGTH: 25
<212> TYPE: DNA
<213> ORGANISM: Artificial sequence
<220> FEATURE:
<223> OTHER INFORMATION: oligonucleotide probe
<400> SEQUENCE: 236
atagataatg tacccagtgc tgagg
#
# 25
<210> SEQ ID NO 237
<211> LENGTH: 25
<212> TYPE: DNA
<213> ORGANISM: Artificial sequence
<220> FEATURE:
<223> OTHER INFORMATION: oligonucleotide probe
<400> SEQUENCE: 237
tcactcatgt ccatcagttt ggaaa
#
# 25
<210> SEQ ID NO 238
<211> LENGTH: 25
<212> TYPE: DNA
<213> ORGANISM: Artificial sequence
<220> FEATURE:
<223> OTHER INFORMATION: oligonucleotide probe
<400> SEQUENCE: 238
tcactcatgt ccgtcagttt ggaaa
#
# 25
<210> SEQ ID NO 239
<211> LENGTH: 25
<212> TYPE: DNA
<213> ORGANISM: Artificial sequence
<220> FEATURE:
<223> OTHER INFORMATION: oligonucleotide probe
<400> SEQUENCE: 239
tcctcagggg gaccagcttt ggctt
#
# 25
<210> SEQ ID NO 240
<211> LENGTH: 25
<212> TYPE: DNA
<213> ORGANISM: Artificial sequence
<220> FEATURE:
<223> OTHER INFORMATION: oligonucleotide probe
<400> SEQUENCE: 240
tcctcagggg gaacagcttt ggctt
#
# 25
<210> SEQ ID NO 241
<211> LENGTH: 25
<212> TYPE: DNA
<213> ORGANISM: Artificial sequence
<220> FEATURE:
<223> OTHER INFORMATION: oligonucleotide probe
<400> SEQUENCE: 241
ggaggaggcg tgcgtggccc aaggg
#
# 25
<210> SEQ ID NO 242
<211> LENGTH: 25
<212> TYPE: DNA
<213> ORGANISM: Artificial sequence
<220> FEATURE:
<223> OTHER INFORMATION: oligonucleotide probe
<400> SEQUENCE: 242
ggaggaggcg tgtgtggccc aaggg
#
# 25
<210> SEQ ID NO 243
<211> LENGTH: 25
<212> TYPE: DNA
<213> ORGANISM: Artificial sequence
<220> FEATURE:
<223> OTHER INFORMATION: oligonucleotide probe
<400> SEQUENCE: 243
gaggaggcgt gcgtggccca agggt
#
# 25
<210> SEQ ID NO 244
<211> LENGTH: 25
<212> TYPE: DNA
<213> ORGANISM: Artificial sequence
<220> FEATURE:
<223> OTHER INFORMATION: oligonucleotide probe
<400> SEQUENCE: 244
gaggaggcgt gcatggccca agggt
#
# 25
<210> SEQ ID NO 245
<211> LENGTH: 25
<212> TYPE: DNA
<213> ORGANISM: Artificial sequence
<220> FEATURE:
<223> OTHER INFORMATION: oligonucleotide probe
<400> SEQUENCE: 245
gtgcgtggcc caagggttgt ggaaa
#
# 25
<210> SEQ ID NO 246
<211> LENGTH: 25
<212> TYPE: DNA
<213> ORGANISM: Artificial sequence
<220> FEATURE:
<223> OTHER INFORMATION: oligonucleotide probe
<400> SEQUENCE: 246
gtgcgtggcc caggggttgt ggaaa
#
# 25
<210> SEQ ID NO 247
<211> LENGTH: 25
<212> TYPE: DNA
<213> ORGANISM: Artificial sequence
<220> FEATURE:
<223> OTHER INFORMATION: oligonucleotide probe
<400> SEQUENCE: 247
aaagaagtta ccaaagaagt ggtga
#
# 25
<210> SEQ ID NO 248
<211> LENGTH: 25
<212> TYPE: DNA
<213> ORGANISM: Artificial sequence
<220> FEATURE:
<223> OTHER INFORMATION: oligonucleotide probe
<400> SEQUENCE: 248
aaagaagtta cctaagaagt ggtga
#
# 25
<210> SEQ ID NO 249
<211> LENGTH: 25
<212> TYPE: DNA
<213> ORGANISM: Artificial sequence
<220> FEATURE:
<223> OTHER INFORMATION: oligonucleotide probe
<400> SEQUENCE: 249
gaattccctt cccgtggtaa atctt
#
# 25
<210> SEQ ID NO 250
<211> LENGTH: 25
<212> TYPE: DNA
<213> ORGANISM: Artificial sequence
<220> FEATURE:
<223> OTHER INFORMATION: oligonucleotide probe
<400> SEQUENCE: 250
gaattccctt cctgtggtaa atctt
#
# 25
<210> SEQ ID NO 251
<211> LENGTH: 25
<212> TYPE: DNA
<213> ORGANISM: Artificial sequence
<220> FEATURE:
<223> OTHER INFORMATION: oligonucleotide probe
<400> SEQUENCE: 251
ttcttcagtg cccgtggtca tcgac
#
# 25
<210> SEQ ID NO 252
<211> LENGTH: 25
<212> TYPE: DNA
<213> ORGANISM: Artificial sequence
<220> FEATURE:
<223> OTHER INFORMATION: oligonucleotide probe
<400> SEQUENCE: 252
ttcttcagtg cctgtggtca tcgac
#
# 25
<210> SEQ ID NO 253
<211> LENGTH: 25
<212> TYPE: DNA
<213> ORGANISM: Artificial sequence
<220> FEATURE:
<223> OTHER INFORMATION: oligonucleotide probe
<400> SEQUENCE: 253
ggctgtcttc acgctgaccc agacc
#
# 25
<210> SEQ ID NO 254
<211> LENGTH: 25
<212> TYPE: DNA
<213> ORGANISM: Artificial sequence
<220> FEATURE:
<223> OTHER INFORMATION: oligonucleotide probe
<400> SEQUENCE: 254
ggctgtcttc acactgaccc agacc
#
# 25
<210> SEQ ID NO 255
<211> LENGTH: 25
<212> TYPE: DNA
<213> ORGANISM: Artificial sequence
<220> FEATURE:
<223> OTHER INFORMATION: oligonucleotide probe
<400> SEQUENCE: 255
gtgattcaga accgtcaaga cggta
#
# 25
<210> SEQ ID NO 256
<211> LENGTH: 25
<212> TYPE: DNA
<213> ORGANISM: Artificial sequence
<220> FEATURE:
<223> OTHER INFORMATION: oligonucleotide probe
<400> SEQUENCE: 256
gtgattcaga actgtcaaga cggta
#
# 25
<210> SEQ ID NO 257
<211> LENGTH: 25
<212> TYPE: DNA
<213> ORGANISM: Artificial sequence
<220> FEATURE:
<223> OTHER INFORMATION: oligonucleotide probe
<400> SEQUENCE: 257
gctgacaagt accgcctaac atatg
#
# 25
<210> SEQ ID NO 258
<211> LENGTH: 25
<212> TYPE: DNA
<213> ORGANISM: Artificial sequence
<220> FEATURE:
<223> OTHER INFORMATION: oligonucleotide probe
<400> SEQUENCE: 258
gctgacaagt actgcctaac atatg
#
# 25
<210> SEQ ID NO 259
<211> LENGTH: 25
<212> TYPE: DNA
<213> ORGANISM: Artificial sequence
<220> FEATURE:
<223> OTHER INFORMATION: oligonucleotide probe
<400> SEQUENCE: 259
ctgacaagta ccgcctaaca tatgc
#
# 25
<210> SEQ ID NO 260
<211> LENGTH: 25
<212> TYPE: DNA
<213> ORGANISM: Artificial sequence
<220> FEATURE:
<223> OTHER INFORMATION: oligonucleotide probe
<400> SEQUENCE: 260
ctgacaagta ccacctaaca tatgc
#
# 25
<210> SEQ ID NO 261
<211> LENGTH: 25
<212> TYPE: DNA
<213> ORGANISM: Artificial sequence
<220> FEATURE:
<223> OTHER INFORMATION: oligonucleotide probe
<400> SEQUENCE: 261
atgcctttga tggctttgat tttgg
#
# 25
<210> SEQ ID NO 262
<211> LENGTH: 25
<212> TYPE: DNA
<213> ORGANISM: Artificial sequence
<220> FEATURE:
<223> OTHER INFORMATION: oligonucleotide probe
<400> SEQUENCE: 262
atgcctttga tgtctttgat tttgg
#
# 25
<210> SEQ ID NO 263
<211> LENGTH: 25
<212> TYPE: DNA
<213> ORGANISM: Artificial sequence
<220> FEATURE:
<223> OTHER INFORMATION: oligonucleotide probe
<400> SEQUENCE: 263
cacatcccat aatggcatgc agttc
#
# 25
<210> SEQ ID NO 264
<211> LENGTH: 25
<212> TYPE: DNA
<213> ORGANISM: Artificial sequence
<220> FEATURE:
<223> OTHER INFORMATION: oligonucleotide probe
<400> SEQUENCE: 264
cacatcccat aagggcatgc agttc
#
# 25
<210> SEQ ID NO 265
<211> LENGTH: 25
<212> TYPE: DNA
<213> ORGANISM: Artificial sequence
<220> FEATURE:
<223> OTHER INFORMATION: oligonucleotide probe
<400> SEQUENCE: 265
cctgggacaa tgacaatgat aagtt
#
# 25
<210> SEQ ID NO 266
<211> LENGTH: 25
<212> TYPE: DNA
<213> ORGANISM: Artificial sequence
<220> FEATURE:
<223> OTHER INFORMATION: oligonucleotide probe
<400> SEQUENCE: 266
cctgggacaa tggcaatgat aagtt
#
# 25
<210> SEQ ID NO 267
<211> LENGTH: 25
<212> TYPE: DNA
<213> ORGANISM: Artificial sequence
<220> FEATURE:
<223> OTHER INFORMATION: oligonucleotide probe
<400> SEQUENCE: 267
ggacctggaa gtactggaag ctgga
#
# 25
<210> SEQ ID NO 268
<211> LENGTH: 25
<212> TYPE: DNA
<213> ORGANISM: Artificial sequence
<220> FEATURE:
<223> OTHER INFORMATION: oligonucleotide probe
<400> SEQUENCE: 268
ggacctggaa gtgctggaag ctgga
#
# 25
<210> SEQ ID NO 269
<211> LENGTH: 25
<212> TYPE: DNA
<213> ORGANISM: Artificial sequence
<220> FEATURE:
<223> OTHER INFORMATION: oligonucleotide probe
<400> SEQUENCE: 269
ccctggacag gcgaggaata cagag
#
# 25
<210> SEQ ID NO 270
<211> LENGTH: 25
<212> TYPE: DNA
<213> ORGANISM: Artificial sequence
<220> FEATURE:
<223> OTHER INFORMATION: oligonucleotide probe
<400> SEQUENCE: 270
ccctggacag gcaaggaata cagag
#
# 25
<210> SEQ ID NO 271
<211> LENGTH: 25
<212> TYPE: DNA
<213> ORGANISM: Artificial sequence
<220> FEATURE:
<223> OTHER INFORMATION: oligonucleotide probe
<400> SEQUENCE: 271
tggacatcat gagagacatc gcctc
#
# 25
<210> SEQ ID NO 272
<211> LENGTH: 25
<212> TYPE: DNA
<213> ORGANISM: Artificial sequence
<220> FEATURE:
<223> OTHER INFORMATION: oligonucleotide probe
<400> SEQUENCE: 272
tggacatcat gaaagacatc gcctc
#
# 25
<210> SEQ ID NO 273
<211> LENGTH: 25
<212> TYPE: DNA
<213> ORGANISM: Artificial sequence
<220> FEATURE:
<223> OTHER INFORMATION: oligonucleotide probe
<400> SEQUENCE: 273
cagacctcag ccatacaacc ctttc
#
# 25
<210> SEQ ID NO 274
<211> LENGTH: 25
<212> TYPE: DNA
<213> ORGANISM: Artificial sequence
<220> FEATURE:
<223> OTHER INFORMATION: oligonucleotide probe
<400> SEQUENCE: 274
cagacctcag ccgtacaacc ctttc
#
# 25
<210> SEQ ID NO 275
<211> LENGTH: 25
<212> TYPE: DNA
<213> ORGANISM: Artificial sequence
<220> FEATURE:
<223> OTHER INFORMATION: oligonucleotide probe
<400> SEQUENCE: 275
ccaaagaaaa ccaggaatct taaga
#
# 25
<210> SEQ ID NO 276
<211> LENGTH: 25
<212> TYPE: DNA
<213> ORGANISM: Artificial sequence
<220> FEATURE:
<223> OTHER INFORMATION: oligonucleotide probe
<400> SEQUENCE: 276
ccaaagaaaa ccgggaatct taaga
#
# 25
<210> SEQ ID NO 277
<211> LENGTH: 25
<212> TYPE: DNA
<213> ORGANISM: Artificial sequence
<220> FEATURE:
<223> OTHER INFORMATION: oligonucleotide probe
<400> SEQUENCE: 277
caaagaaaac caggaatctt aagaa
#
# 25
<210> SEQ ID NO 278
<211> LENGTH: 25
<212> TYPE: DNA
<213> ORGANISM: Artificial sequence
<220> FEATURE:
<223> OTHER INFORMATION: oligonucleotide probe
<400> SEQUENCE: 278
caaagaaaac cacgaatctt aagaa
#
# 25
<210> SEQ ID NO 279
<211> LENGTH: 25
<212> TYPE: DNA
<213> ORGANISM: Artificial sequence
<220> FEATURE:
<223> OTHER INFORMATION: oligonucleotide probe
<400> SEQUENCE: 279
gtgactctca gcgagcctca atgct
#
# 25
<210> SEQ ID NO 280
<211> LENGTH: 25
<212> TYPE: DNA
<213> ORGANISM: Artificial sequence
<220> FEATURE:
<223> OTHER INFORMATION: oligonucleotide probe
<400> SEQUENCE: 280
gtgactctca gcaagcctca atgct
#
# 25
<210> SEQ ID NO 281
<211> LENGTH: 25
<212> TYPE: DNA
<213> ORGANISM: Artificial sequence
<220> FEATURE:
<223> OTHER INFORMATION: oligonucleotide probe
<400> SEQUENCE: 281
tgtctgcggg agccgatttc atcat
#
# 25
<210> SEQ ID NO 282
<211> LENGTH: 25
<212> TYPE: DNA
<213> ORGANISM: Artificial sequence
<220> FEATURE:
<223> OTHER INFORMATION: oligonucleotide probe
<400> SEQUENCE: 282
tgtctgcggg agtcgatttc atcat
#
# 25
<210> SEQ ID NO 283
<211> LENGTH: 25
<212> TYPE: DNA
<213> ORGANISM: Artificial sequence
<220> FEATURE:
<223> OTHER INFORMATION: oligonucleotide probe
<400> SEQUENCE: 283
tgaccagtga agaaagtgtc tttga
#
# 25
<210> SEQ ID NO 284
<211> LENGTH: 25
<212> TYPE: DNA
<213> ORGANISM: Artificial sequence
<220> FEATURE:
<223> OTHER INFORMATION: oligonucleotide probe
<400> SEQUENCE: 284
tgaccagtga agcaagtgtc tttga
#
# 25
<210> SEQ ID NO 285
<211> LENGTH: 25
<212> TYPE: DNA
<213> ORGANISM: Artificial sequence
<220> FEATURE:
<223> OTHER INFORMATION: oligonucleotide probe
<400> SEQUENCE: 285
gcctatacag tcactttttt ttttt
#
# 25
<210> SEQ ID NO 286
<211> LENGTH: 25
<212> TYPE: DNA
<213> ORGANISM: Artificial sequence
<220> FEATURE:
<223> OTHER INFORMATION: oligonucleotide probe
<400> SEQUENCE: 286
ctgggaccac agcgcccgcc actac
#
# 25
<210> SEQ ID NO 287
<211> LENGTH: 25
<212> TYPE: DNA
<213> ORGANISM: Artificial sequence
<220> FEATURE:
<223> OTHER INFORMATION: oligonucleotide probe
<400> SEQUENCE: 287
gcctatacag tcacttttat gtggt
#
# 25

We claim:

1. A method of detecting genetic predisposition to venous thrombosis (VT) in a subject, comprising:
determining whether the subject has one or more mutations or polymorphisms in at least eight VT-related molecules, wherein the at least eight venous thrombosis molecules comprise antithrombin III (AT III), protein C, protein S, fibrinogen, factor V (FV), prothrombin (factor I), methylenetetrahydrofolate reductase (MTHFR) and angiotensin 1-converting enzyme (ACE), and wherein the presence of one or more mutations or polymorphisms indicates that the subject has a genetic predisposition for venous thrombosis.

2. The method of claim 1, wherein the one or more mutations or polymorphisms comprise one or more mutations or polymorphisms listed in Table 1.

3. The method of claim 1, wherein the method comprises determining whether the subject has one or more mutations or polymorphisms in at least 10 of the mutations or polymorphisms listed in Table 1.

4. The method of claim 1, wherein the method comprises determining whether the subject has one or more mutations or polymorphisms in at least 50 of the mutations or polymorphisms listed in Table 1.

5. The method of claim 1, wherein the method comprises determining whether the subject has one or more mutations or polymorphisms in at least 143 of the mutations or polymorphisms listed in Table 1.

6. The method of claim 1, wherein the method comprises determining whether the subject has one or more mutations or polymorphisms in no more than 10 of the mutations or polymorphisms listed in Table 1.

7. The method of claim 2, wherein the one or more mutations or polymorphisms comprise AT III deficiency, PC deficiency, PS deficiency, fibrinogen Thr312Ala polymorphism, FV Leiden (G1691A) polymorphism, FV G1628 polymorphism, FV A4070G polymorphism, prothrombin G20210A polymorphism, MTHFR C677T and ACE intron 16, 288 bp insertion/deletion polymorphism.

8. The method of claim 1, wherein the method provides a probability of developing VT of at least 98% in Caucasians, at least 85% in Asians, and at least 87% in Africans.

9. The method of claim 1, wherein the method comprises determining whether the subject has one or more mutations or polymorphisms in at least eight VT-related molecules.

10. The method of claim 1, wherein the at least eight VT-related molecules comprise nucleic acid molecules.

11. The method of claim 10, wherein the nucleic acid molecules are amplified from the subject, thereby generating amplification products, and wherein the amplification products are hybridized with oligonucleotide probes that detect the one or more mutations or polymorphisms.

12. The method of claim 11, wherein hybridizing the oligonucleotides comprises:
incubating the amplification products with the oligonucleotide probes for a time sufficient to allow hybridization between the amplification products and oligonucleotide probes, thereby forming amplification products: oligonucleotide probe complexes; and
analyzing the amplification products: oligonucleotide probe complexes to determine if the amplification products comprise one or more mutations or polymorphisms in the VT-related nucleic acids, wherein the presence of one or more mutations or polymorphisms indicates that the subject has a genetic predisposition for VT.

13. The method of claim 12, wherein analyzing the amplification products:oligonucleotide probe complexes comprises determining an amount of nucleic acid hybridization, and wherein a greater amount of hybridization to one or more of the mutated sequences, as compared to an amount of hybridization to a corresponding wild-type sequence, indicates that the subject has a genetic predisposition for VT.

14. The method of claim 12, wherein analyzing the amplification products:oligonucleotide probe complexes includes detecting and quantifying the complexes.

15. The method of claim 11, wherein the oligonucleotide probes are present on an array substrate.

16. The method of claim 15, wherein the array further comprises oligonucleotide probes complementary to wild-type VT-related nucleic acid molecules.

17. The method of claim 16, wherein the wild-type VT-related nucleic acid molecules comprise oligonucleotide probes complementary to wild-type AT III, wild-type protein C, wild-type protein S, wild-type fibrinogen, wild-type factor V, wild-type factor II, wild-type MTHFR and wild-type ACE nucleic acid sequences.

18. The method of claim 1, wherein the at least eight VT-related molecules consist of sequences from AT III, protein C, protein S, fibrinogen, factor V, factor II, MTHFR and ACE.

19. The method of claim 1, wherein the subject is in a group potentially at risk of developing a venous thrombosis.

20. The method of claim 19, wherein the subject is pregnant, is in puerperium, is using oral contraceptives or hormone replacement therapy, has previous thrombosis history, has or will undergo prolonged immobilization, has a myeloproliferative disorder, has a malignancy, has or will undergo surgery, has a bone fracture, is of advanced age, has antiphospholipid antibodies, or combinations thereof.

21. The method of claim 11, wherein the nucleic acid molecules obtained from the subject are obtained from serum.

22. A method of detecting genetic predisposition to VT in a subject, comprising:
applying amplification products to an array, wherein the array comprises oligonucleotide probes complementary to mutated AT III, mutated protein C, mutated protein S, mutated fibrinogen, mutated factor V, mutated factor II, mutated MTHFR and mutated ACE sequences, and wherein the amplification products comprise nucleic acid sequences from AT III, protein C, protein S, fibrinogen, factor V, factor II, MTHFR and ACE, obtained from the subject;
incubating the amplification products with the array for a time sufficient to allow hybridization between the amplification products and oligonucleotide probes, thereby forming amplification products: oligonucleotide probe complexes; and
analyzing the amplification products: oligonucleotide probe complexes to determine if the amplification products comprise one or more mutations or polymorphisms in the AT III, protein C, protein S, fibrinogen, factor V, factor 11, MTHFR or ACE sequences, wherein the presence of one or more mutations or polymorphisms indicates that the subject has a genetic predisposition for VT.

23. A method of selecting a venous thrombosis (VT) therapy, comprising:
detecting a mutation or polymorphism in at least one VT-related molecule of a subject, using the method of claim 1; and
if such mutation or polymorphism is identified, selecting a treatment to avoid or reduce VT, or to delay the onset of VT.

24. The method of claim 23, further comprising administering the selected treatment to the subject.

25. The method of claim 24, wherein the selected treatment comprises treating the subject with an anticoagulant agent.

26. An array comprising oligonucleotide probes complementary to wild-type gene sequences, mutated gene sequences, or both, wherein the gene sequences comprise coding or non-coding sequences from AT III, protein C, protein S, fibrinogen, factor V, factor II, MTHFR and ACE genes.

27. The array of claim 26, wherein the mutated gene sequences comprise ten or more mutations or polymorphisms listed in Table 1.

28. The array of claim 27, wherein the mutated gene sequences consist essentially of the mutations or polymorphisms listed in Table 1.

29. A method of detecting a genetic predisposition to venous thrombosis (VT) in a subject, comprising:
applying amplification products to the array of claim 13, wherein the amplification products comprise amplified nucleic acids obtained from the subject, wherein the nucleic acids comprise coding or non-coding sequences from AT III, protein C, protein S, fibrinogen, factor V, factor II, MTHFR and ACE.
incubating the amplification products with the array for a time sufficient to allow hybridization between the amplification products and oligonucleotide probes, thereby forming amplification products: oligonucleotide probe complexes; and
analyzing the amplification products: oligonucleotide probe complexes to determine if the amplification products comprise one or more mutations or polymorphisms in the AT III, protein C, protein S, fibrinogen, factor V, factor II, MTHFR, or ACE genes, wherein the presence of one or more mutations or polymorphisms indicates that the subject has a genetic predisposition for VT.

30. A kit for detecting a genetic predisposition to venous thrombosis (VT) in a subject, comprising:
a solid phase nucleic acid array comprising a plurality of oligonucleotide probes chemically linked to a solid polymeric support surface in a predetermined pattern, wherein the oligonucleotide probes are capable of hybridizing under stringent conditions to one or more nucleic acid molecules having VT-related mutations or polymorphisms in AT III, protein C, protein S, fibrinogen, factor V, factor II, MTHFR and ACE genes.

31. The kit of claim 30, wherein the oligonucleotides comprise SEQ ID NOS: 1-287.

32. The kit of claim 30, further comprising primers for amplifying nucleic acid molecules obtained from the subject to obtain amplification products, in separate packaging, wherein the amplification products comprise sequences from AT III, protein C, protein S, fibrinogen, factor V, factor II, MTHFR, and ACE genes;

33. The kit of claim 30, further comprising an amplification enzyme, in separate packaging.

34. The kit of claim 30, further comprising a buffer solution, in separate packaging.

35. The kit of claim 30, wherein the array further comprises oligonucleotides capable of hybridizing under stringent conditions to a wild-type AT m, wild-type protein C, wild-type protein S, wild-type fibrinogen, wild-type factor V, wild-type factor II, wild-type MTHFR, and wild-type ACE.
